(19) United States
(12) Patent Application Publication
(10) Pub. No.: US 2020/0023189 A1
(43) Pub. Date: Jan. 23, 2020

(54) BRAIN COMPUTER INTERFACE SYSTEMS AND METHODS OF USE THEREOF

(71) Applicant: Inner Cosmos LLC, New Haven, CT (US)

(72) Inventors: Meron GRIBETZ, New York City, NY (US); Eric Claude LEUTHARDT, St. Louis, MO (US)

(73) Assignee: Inner Cosmos LLC, New Haven, CT (US)

(21) Appl. No.: 16/469,165

(22) PCT Filed: Dec. 14, 2017
(86) PCT No.: PCT/IB2017/057952

§ 371 Date: Jun. 13, 2019

Related U.S. Application Data
(60) Provisional application No. 62/470,900, filed on Mar. 14, 2017.
(60) Provisional application No. 62/433,946, filed on Dec. 14, 2016.

(51) Int. Cl. A61N 1/372; A61N 1/36; A61N 1/05

(52) U.S. Cl. CPC A61N 1/37252; A61N 1/36196; A61N 1/0534; A61N 1/0531; A61N 1/36135; A61N 1/36096; A61N 1/36025; A61N 1/36031; A61N 1/36092

(57) ABSTRACT

A brain computer interface (BCI) system for modulating cognitive performance. The system includes one or more electrode sets for sensing signals associated with neuronal electrical activity in one or more cortical regions of the user and for providing stimulating signals to one or more target brain regions, at least one processor/controller in communication with the one or more electrode sets, and at least one power source. The processor/controller is programmed to process signals sensed in the one or more cortical regions for detecting an indication associated with an intention to perform a cognitive task and/or the presentation of a cognitive task and/or the performing of a cognitive task, and to control the stimulating of the one or more target brain regions responsive to the detecting of the indication for modulating the cognitive performance of the user. The target brain regions may include cortical regions, deep brain structures, and combinations thereof.

### RELATED APPLICATIONS

This application claims the benefit of priority of U.S. Provisional Patent Application No. 62/433,946 filed Dec. 14, 2016, and of U.S. Provisional Patent Application No. 62/470,900 filed Mar. 14, 2017, the contents of which are incorporated herein by reference in their entirety.

### FIELD AND BACKGROUND OF THE INVENTION

The present invention, in some embodiments thereof, relates to systems for augmenting and/or enhancing and/or improving cognitive performance of a user.

Such systems may enhance or augment, inter alia, memory, working memory (WM) learning and attention focusing in normal users and may be used to improve the cognitive performance of patients suffering from neurological disorders and/or neuropsychiatric disorders and/or psychiatric disorders associated with impaired or reduced cognitive function.

Brain computer interfaces (BCIs) are devices or systems used for interacting with the brain and other types of neural tissues for performing sensing and/or recording of neuronal tissues and for stimulating neurons in such tissues. Such BCIs may be used for sensing/recording signals (typically, transient electrical signals such as voltage or current signals) associated with neuronal activity. Currently, most BCIs include multiple electrically conducting electrodes, often arranged as a two dimensional (2D) or three-dimensional (3D) electrode arrays. Such electrode arrays may be used to sense electrical signals associated with neuronal activity and/or stimulate neurons by passing suitable electrical currents through the electrodes.

Some electrodes or electrode sets included in BCIs may be non-invasive such as, for example extra-cranial EEG electrode arrays, while other electrodes or sets of electrodes may be invasive such as flexible intracranial electrocorticogram electrode arrays placed on the cortical surface. Other invasive electrode arrays may be inserted into the cortical tissue (such as, for example Utah arrays which may be typically placed on the cortical surface and inserted superficially into the first few millimeters of cortical tissue). Still other electrode arrays may be disposed on stents. Such stents may be inserted through the vasculature using minimally invasive methods, and may be disposed in blood vessels of the brain close to relevant brain regions.

The signals sensed and/or recorded by electrode set(s) of the BCI systems of the present applications may include, inter alia, single neuron extracellular recorded action potentials (spikes), extracellular recorded neuronal action potentials from single or multiple neurons, local field potentials (LFP) from single or multiple neurons, surface recorded field potentials resulting from summed activity of neuronal assemblies, Ecog signals and extra-cranially recorded EEG signal.

Significant advances have been recently achieved in the use of such BCIs used for sensing/recording and/or stimulation for sensing/recording neural activity from the motor cortex and processing the sensed signals to control the operation/movements of a prosthesis replacing a missing limb in patients. Advances were also made in using signals recorded from the motor cortex of quadriplegic patients to enable such patients to control a motorized wheelchair or to control other devices such as a computer which may perform various functions for assisting such patient.

Other uses for such BCIs for assisting blind patients include using images of a field of view acquired by an external video camera and processed to control electrical stimulation of the primary visual cortex of the blind patient by a flexible Ecog electrode array BCI placed on the surface of the visual cortex, resulting in the perception of phosphenes by the blind patient which may assist patient's navigation, object identification and obstacles avoidance.

The Ventral Tegmental Area (VTA) is part of the midbrain, lying close to the substantia nigra and the red nucleus. It is rich in dopamine and serotonin neurons, and is part of two major dopamine pathways: 1. the mesolimbic pathway, which connects the VTA to the nucleus accumbens; 2. the mesocortical pathway, which connects the VTA to cortical areas in the frontal lobes. The VTA is considered to be part of the pleasure system, or reward circuit, one of the major sources of incentive and behavioral motivation and as such may be relevant to reinforced learning methods and systems disclosed hereinafter. Activities that produce pleasure tend to activate the ventral tegmentum, and psychostimulant drugs (such as cocaine) directly target this area. Hence, it is widely implicated in neurobiological theories of addiction. It is also shown to process various types of emotion and security motivation, where it may also play a role in avoidance and fear conditioning.

The Prefrontal Cortex (PFC) is the anterior part of the frontal lobes of the brain, lying in front of the motor and premotor areas. Cytoarchitectonically, it is defined by the presence of an internal granular layer IV (in contrast to the agranular premotor cortex). Divided into the lateral, orbitofrontal and medial prefrontal areas, this brain region has been implicated in planning complex cognitive behaviors, personality expression and moderating correct social behavior. The basic activity of this brain region is considered to be the orchestration of thoughts and actions in accordance with internal goals. The most typical neurologic term for functions carried out by the pre-frontal cortex area is Executive Function. Executive Function relates to abilities to differentiate between conflicting thoughts, determine good and bad, better and best, same and different, future consequences of current activities, working toward a defined goal, prediction of outcomes, expectation based on actions, and social “control” (the ability to suppress urges that, if not suppressed, could lead to socially unacceptable or illegal outcomes). Many authors have indicated an integral link between a person's personality and the functions of the prefrontal cortex.

The dorsolateral prefrontal cortex (DLPFC) is one of the most recently evolved parts of the human brain that undergoes an extremely prolonged period of maturation that lasts until adulthood. DLPFC is not an anatomical structure, but rather a functional one. This region lies in the middle frontal gyms of humans (i.e., lateral part of Brodmann's area (BA) 9 and 46 and in macaque monkeys, this region is around the principal sulcus (i.e., in Walker's area 46). Other sources propose that the DLPFC is attributed anatomically to BA 9 and 46 and BA 8, 9 and 10.

The DLPFC is connected to the orbitofrontal cortex, and to a variety of brain areas, which include the thalamus, parts of the basal ganglia (specifically, the dorsal caudate nucleus), the hippocampus, and primary and secondary association areas of the neocortex, including posterior temporal, parietal, and occipital areas. The DLPFC is the end point for the dorsal pathway (dorsal stream) that tells the brain how to interact with the stimuli. The DLPFC is also the highest cortical area that is involved in motor planning, organization and regulation

On the other hand, the ventrolateral prefrontal cortex (located more inferior/ventral to DLPFC) is the end point of the ventral pathway (ventral stream) that brings information about the stimuli's characteristics. An important function of the DLPFC is the executive functions, such as working memory, cognitive flexibility, planning, inhibition, and abstract reasoning. However, DLPFC is not exclusively responsible for the executive functions. All complex mental activity requires the additional cortical and subcortical circuits with which the DLPFC is connected.

A couple of tasks have been very prominent in the research on the DLPFC, such as the A-not-B task, the delayed response task and object retrieval tasks. The behavioral task that is most strongly linked to the DLPFC is the combined A-not-B/delayed response task, in which the subject has to find a hidden object after a certain delay. This task requires holding the information in mind (working memory) which is believed to be one of the functions of DLPFC. The importance of DLPFC for working memory was strengthened by studies with adult macaques. Lesions that destroyed DLPFC disrupted the macaques' performance of the A-not-B/delayed response task, whereas lesions to other brain parts did not impair their performance on this task.

The DLPFC is not required for the memory of a single item. Thus, damage to the DLPFC does not impair recognition memory. Nevertheless, if two items must be compared from memory, the involvement of DLPFC is required. People with damaged DLPFC are not able to identify a picture they had seen, after some time, when given the opportunity to choose from two pictures. Moreover, these subjects also failed in Wisconsin Card-Sorting Test as they lose track of the currently correct rule and persistently organize their cards in the previously correct rule. Likewise, the DLPFC is most frequently related to the dysfunction of drive, attention and motivation. Patients with minor DLPFC damage display disinterest in their surroundings and are deprived of spontaneity in language as well as behavior. Patients may also be less alert to people and events they know. Damage to this region in a person also leads to the lack of motivation to do things for themselves and/or for others.

Working memory is the system that actively holds multiple pieces of transitory information in the mind, where they can be manipulated. The DLPFC is important for working memory. Reduced activity in the DLPFC correlates to poor performance on working memory tasks. However, other areas of the brain are involved in working memory as well.

There is an ongoing discussion and it is not yet clear if the DLPFC is specialized in a type of working memory, namely computational mechanisms for monitoring and manipulating generic items, or if it is more specialized to handle a more specific subset of items, namely visuospatial information, which makes it possible to mentally represent coordinates within the spatial domain.

The locus ceruleus (LC), also spelled locus caeruleus or locus coeruleus (Latin for ‘the blue spot’), is a nucleus in the brain stem responsible for physiological responses to stress and panic. The locus ceruleus (or “LC”) resides on the dorsal wall of the upper pons, under the cerebellum in the caudal midbrain, surrounded by the fourth ventricle. This nucleus is one of the main sources of norepinephrine in the brain, and is composed of mostly medium-sized neurons. Melanin granules inside the LC contribute to its blue color; it is thereby also known as the nucleus pigmentosus pontis, meaning “heavily pigmented nucleus of the pons”. The neuromelanin is formed by the polymerization of norepinephrine and is analogous to the black dopamine-based neuromelanin in the substantia nigra. The projections of this nucleus reach far and wide, innervating the spinal cord, the brain stem, cerebellum, hypothalamus, the thalamic relay nuclei, the amygdala, the basal telencephalon, and the cortex. The norepinephrine from the LC has an excitatory effect on most of the brain, mediating arousal and priming the brain's neurons to be activated by stimuli. It has been said, that a single noradrenergic neuron can innervate, via its branches, the entire cerebral cortex.

The Hippocampus is a part of the brain located inside the temporal lobe (humans have two hippocampi, one in each side of the brain). It forms a part of the limbic system and plays a part in memory and navigation. The name derives from its curved shape in coronal sections of the brain, which to some resembles a seahorse (Greek: hippokampos). In Alzheimer's disease, the hippocampus becomes one of the first regions of the brain to suffer damage; memory problems and disorientation appear amongst the first symptoms. Damage to the hippocampus can also result from oxygen starvation (anoxia) and encephalitis. In the anatomy of animals, the hippocampus is among the phylogenetically oldest parts of the brain. The hippocampal emergence from the archipallium is most pronounced in primates and Cetacean sea mammals. Nonetheless, in primates the hippocampus occupies less of the telencephalon in proportion to cerebral cortex among the youngest species, especially humans. The significant development of hippocampal volume in primates correlates more with overall increase of brain mass than with neocortical development.

Although there is a lack of consensus relating to terms describing the hippocampus and the adjacent cortex, the term hippocampal formation generally applies to the dentate gyms, fields CA1-CA3 (or CA4, frequently called the hilus and considered part of the dentate gyrus), and the subiculum. The CA1 and CA3 fields make up the hippocampus proper.

Information flow through the hippocampus proceeds from dentate gyrus to CA3 to CA1 to the subiculum, with additional input information at each stage and outputs at each of the two final stages. CA2 represents only a very small portion of the hippocampus and its presence is often ignored in accounts of hippocampal function, though it is notable that this small region seems unusually resistant to conditions that usually cause large amounts of cellular damage, such as epilepsy.

The perforant path, which brings information primarily from entorhinal cortex (but also perirhinal cortex, among others), is generally considered the main source of input to the hippocampus. Layer II of entorhinal cortex (EC) brings input to the dentate gyrus and field CA3, while EC layer III brings input to field CA1 and the subiculum. The main output pathways of the hippocampus are the perforant path, the cingulum bundle, and the fimbria/fornix, which all arise from field CA1 and the subiculum.

Perforant path input from EC layer II enters the dentate gyrus and is relayed to region CA3 (and to mossy cells, located in the hilus of the dentate gyrus, which then send information to distant portions of the dentate gyrus where the cycle is repeated). Region CA3 combines this input with signals from EC layer II and sends extensive connections within the region and also sends connections to region CA1 through a set of fibers called the Schaffer collaterals. Region CA1 receives input from region CA3 as well as EC layer III and then projects to the subiculum as well as sending information along the aforementioned output paths of the hippocampus. The subiculum is the final stage in the pathway, combining information from the CA1 projection and EC layer III to also send information along the output pathways of the hippocampus. It is widely accepted that each of these regions has a unique functional role in the information processing of the hippocampus, but to date the specific contribution of each region is poorly understood.

Psychologists and neuroscientists dispute the precise role of the hippocampus, but, in general, agree that it has an essential role in the formation of new memories about experienced events (episodic or autobiographical memory). Some researchers prefer to consider the hippocampus as part of a larger medial temporal lobe memory system responsible for general declarative memory (memories that can be explicitly verbalized—these would include, for example, memory for facts in addition to episodic memory).

Some evidence supports the idea that, although these forms of memory often last a lifetime, the hippocampus ceases to play a crucial role in the retention of the memory after a period of consolidation. Damage to the hippocampus usually results in profound difficulties in forming new memories (anterograde amnesia), and normally also affects access to memories prior to the damage (retrograde amnesia). Although the retrograde effect normally extends some years prior to the brain damage, in some cases older memories remain—this sparing of older memories leads to the idea that consolidation over time involves the transfer of memories out of the hippocampus to other parts of the brain. However, experimentation has difficulties in testing the sparing of older memories; and, in some cases of retrograde amnesia, the sparing appears to affect memories formed decades before the damage to the hippocampus occurred, so its role in maintaining these older memories remains controversial.

Damage to the hippocampus does not affect some aspects of memory, such as the ability to learn new skills (playing a musical instrument, for example), suggesting that such abilities depend on a different type of memory (procedural memory) and different brain regions. Moreover, there is evidence suggesting that patient HM (who had his medial temporal lobes removed bilaterally as a treatment for epilepsy) can form new semantic memories.

Some evidence implicates the hippocampus in storing and processing spatial information. Studies in rats have shown that neurons in the hippocampus have spatial firing fields. These cells are called place cells. Some cells fire when the animal finds itself in a particular location, regardless of direction of travel, while most are at least partially sensitive to head direction and direction of travel. In rats, some cells, termed splitter cells, may alter their firing depending on the animal's recent past (retrospective) or expected future (prospective). Different cells fire at different locations, so that, by looking at the firing of the cells alone, it becomes possible to tell where the animal is. Place cells have now been found in humans involved in finding their way around in a virtual reality town. The findings resulted from research with individuals that had electrodes implanted in their brains as a diagnostic part of surgical treatment for serious epilepsy.

The discovery of place cells led to the idea that the hippocampus might act as a cognitive map—a neural representation of the layout of the environment. Recent evidence has cast doubt on this perspective, indicating that the hippocampus might be crucial for more fundamental processes within navigation. Regardless, studies with animals have shown that an intact hippocampus is required for simple spatial memory tasks (for instance, finding the way back to a hidden goal).

Without a fully-functional hippocampus, humans may not successfully remember the places they have been to and how to get where they are going. Researchers believe that the hippocampus plays a particularly important role in finding shortcuts and new routes between familiar places. Some people exhibit more skill at this sort of navigation than do others, and brain imaging shows that these individuals have more active hippocampi when navigating.

The Amygdala (Latin, corpus amygdaloideum) is an almond-shaped set of neurons located deep in the brain's medial temporal lobe. The amygdala, that has been shown to play a key role in the processing of emotions, forms part of the limbic system. In humans and other animals, this subcortical brain structure is linked to both fear responses and pleasure. The size of the amygdale is positively correlated with aggressive behavior across species. In humans, it is the most sexually dimorphic brain structure, and shrinks by more than 30% in males upon castration. Conditions such as anxiety, autism, depression, post-traumatic stress disorder, and phobias are suspected of being linked to abnormal functioning of the amygdala, owing to damage, developmental problems, or neurotransmitter imbalance. The amygdala is actually several separately functioning nuclei that anatomists group together by the proximity of the nuclei to one another. Key among these nuclei are the basolateral complex, the centromedial nucleus, and the cortical nucleus.

The basolateral complex can be further subdivided in to the lateral, the basal, and accessory basal nuclei. The lateral amygdala, which is afferent to both the rest of the basolateral complex as well as the centromedial nucleus, receives input from the sensory systems and is necessary for fear conditioning in rats. The centromedial nucleus is the main output for the basolateral complex, and is involved in emotional arousal in rats and cats. The amygdala sends outputs to the hypothalamus for activation of the sympathetic nervous system, the reticular nucleus for increased reflexes, the nuclei of the trigeminal nerve and facial nerve for facial expressions of fear, and the ventral tegmental area, locus ceruleus, and laterodorsal tegmental nucleus for activation of dopamine, norepinephrine and epinephrine. The cortical nucleus is involved in olfaction and pheromone-processing. It receives input from the olfactory bulb and olfactory cortex.

A key function of the amygdala in complex vertebrates, including humans, is the forming and storing of memories of emotional events. Damage to the amygdala may impair both the acquisition and expression of Pavlovian fear conditioning, a form of classical conditioning of emotional responses. Considerable research indicates that, during fear conditioning, sensory stimuli reach the basolateral complex, particularly the lateral nucleus of the amygdala, where they become associated. The association between stimuli and the aversive events they predict may be mediated by long-term potentiation, a form of long-lasting synaptic plasticity. Memories of emotional experiences stored in lateral nucleus synapses elicit fear behavior though connections with the central nucleus of the amygdala, a center involved in the genesis of many fear responses, including freezing (immobility), tachycardia (rapid heartbeat), increased respiration, and stress-hormone release.

The amygdala also plays a role in appetitive (positive) conditioning. It seems that distinct neurons respond to positive and negative stimuli, but there is no clustering of these distinct neurons into clear anatomical nuclei. The suppression of learned fear responses is an important goal of therapeutic interventions for disorders of fear and anxiety, such as post-traumatic stress disorder and phobias, in humans. Evidence suggests that the amygdala is involved not only in fear conditioning, but also in the extinction of fear responses. Extinction, which occurs when fear signals are presented alone several times, yields a reduction in fear responses to those signals. Extinction training does not eliminate the fear memory, however; it is accompanied by new learning that inhibits the original fear. It is interesting to note that extinction learning (at least for fear responses) may also require synaptic plasticity in the amygdala. Systematic desensitization is a type of behavioral therapy for anxiety that relies on extinction learning.

The amygdala also plays a key role in the modulation of memory consolidation. Following any learning event, the long-term memory for the event is not instantaneously formed. Rather, information regarding the event is slowly put into long-term storage over time, a process referred to as memory consolidation, until it reaches a relatively permanent state. During the consolidation period, the memory can be modulated. In particular, it appears that emotional arousal following the learning event influences the strength of the subsequent memory for that event. Greater emotional arousal following a learning event enhances a person's retention of that event. Experiments have shown that administration of stress hormones to individuals immediately after they learn something enhances their retention when they are tested two weeks later.

The amygdala, especially the basolateral amygdala, plays a key role in mediating the effects of emotional arousal on the strength of the memory for the event, as shown by many laboratories including that of James McGaugh. These laboratories have trained animals on a variety of learning tasks and found that drugs injected into the amygdala after training affect the animals' subsequent retention of the task. These tasks include basic Pavlovian tasks such as inhibitory avoidance (where a rat learns to associate a mild foot shock with a particular compartment of an apparatus) and more complex tasks such as spatial or cued water maze (where a rat learns to swim to a platform to escape the water). If a drug that activates the amygdala is injected into the amygdala, the animal has better memory for the training in the task. If a drug that inactivates the amygdala is injected into it, the animal has impaired memory for the task. Despite the importance of the amygdala in modulating memory consolidation, however, learning can occur without it, though such learning appears to be impaired, as in fear conditioning impairments following amygdala damage.

Evidence from work with humans indicates that the amygdala plays a similar role. Amygdala activity at the time of encoding information correlates with retention for that information. However, this correlation depends on the relative “emotionalness” of the information. More emotionally-arousing information increases amygdala activity, and that activity correlates with retention.

Experiments with rats also suggest that the amygdala is involved in learning about various cues with the consumption of drugs of abuse. It is well-known that one of the major problems in drug addiction is that drug-associated cues induce significant craving in individuals, even if the individuals have not taken the drugs in a long time. The basolateral amygdala appears to play a key role in the initial learning of the association between cues and the rewards that they predict. In addition, inactivation of the basolateral amygdala prevents the ability of cues to induce reinstatement in rats in a drug self-administration paradigm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### SUMMARY OF THE INVENTION

There is therefore provided in accordance with some embodiments of the present application a brain computer interface (BCI) system for augmenting and/or assisting and/or improving cognitive performance of a user. The system includes one or more electrode sets for sensing signals associated with neuronal electrical activity in one or more cortical regions of the user and for providing stimulating signals to one or more target brain regions. The system also includes at least one processor/controller in communication with the one or more electrode sets. The at least one processor/controller is programmed to process signals sensed in the one or more cortical regions for detecting an indication associated with an intention to perform a cognitive task and/or the presentation of a cognitive task and/or the performing of a cognitive task, and to control the stimulating of the one or more target brain regions responsive to the detecting of the indication for augmenting and/or assisting, and/or improving the cognitive performance of the user. The system also includes at least one power source for energizing the BCI system.

In accordance with some embodiments of the systems, the one or more target brain regions are selected from the group consisting of one or more deep brain structures of the user, one or more cortical regions of the user, and a combination of one or more cortical regions and one or more deep brain structures.

In accordance with some embodiments of the systems, the one or more cortical regions include one or more of prefrontal cortex (PFC), a part of the PFC, a dorsolateral prefrontal cortex (DLPFC), a part of the DLPFC, a temporoparietal cortex (TPC), a part of the TPC, an inferior frontal gyrus (IFG), a part of the IFG, the temporal parietal junction (TPJ), a part of the TPJ, and any combinations thereof.

In accordance with some embodiments of the systems, the one or more deep brain structures are selected from ventral tegmental area (VTA), striatum, caudate nucleus, putamen, nucleus accumbens (NA), locus ceruleus, hippocampus, amygdala, a deep brain structure of the meso-limbic system, a deep brain structure functionally participating in enhancing and/or facilitating learning, memory and attention focusing, a subcortical region of the brain, a substantia nigra, a dorsal striatum, a part of the limbic structures within a mesocortical system, a part of a nigrostriatal system, a part of tuberoinfundibular system, fornix, nucleus basalis of Meynert (NBM), anterior caudate nucleus, dorsal striatum, anterior thalamic nucleus, central thalamus, lateral hypothalamus, subgenual cingulated region (BA 25), enthorinal cortex, perforant path, medial frontal lobe, subthalamic nucleus and any combinations thereof.

In accordance with some embodiments of the systems, the cognitive performance includes one or more of, attention focusing performance, memory performance, short term memory performance, learning performance, memory retrieval performance, working memory performance and any combinations thereof.

In accordance with some embodiments of the systems, the cognitive task is selected from, an attention focusing task, an attention sustaining task, a memorizing task, a short term memory requiring task, a learning task, a memory retrieval task, and any combinations thereof.

In accordance with some embodiments of the systems. The system according to any of the preceding claims, wherein the user is selected from a normal user and a user having a neurological disorder, a psychiatric disorder, or a neuro-psychiatric disorder.

In accordance with some embodiments of the systems, the neurological disorder or psychiatric disorder or psychiatric-neurological disorder is selected from, ADHD, ADD, a learning deficiency, an attention related deficiency or dysfunction, amnesia, a memory related dysfunction, anxiety, depression, traumatic brain injury, stroke, dementia, neurodegenerative disorder, and any combinations thereof.

In accordance with some embodiments of the systems, the one or more electrode sets are configured for sensing neuronal electrical activity in one or more additional cortical regions of the user and/or for stimulating neurons in the one or more additional cortical regions selected from a visual cortical region, a region of the primary visual cortex (V1), the medial temporal lobe of the visual cortex, a region of the motor cortex, a region of the pre-motor cortex, a region of the somato-sensory cortex, a region of the auditory cortex, the mesial surface of the right cortical occipital lobe, the associative cortex, the primary visual cortex, other areas of the visual cortex, the auditory cortex, the motor cortex, BA 17, BA 18, BA 19, BA 7, BA 6, BA 5, BA 4 and any combinations thereof.

In accordance with some embodiments of the systems, the one or more electrode sets are selected from, non-invasive electrode sets, invasive electrode sets, and any combinations thereof. In accordance with some embodiments of the systems, the one or more electrode sets is selected from the following electrode sets:

1) at least one sensing and stimulating electrode set configured for performing sensing in the one or more cortical regions and for stimulating one or more of the target brain regions.

2) At least one sensing electrode set configured for performing sensing in the one or more cortical regions and at least one stimulating electrode set for stimulating one or more of the target brain regions.

3) At least one electrode set configured for performing sensing in one or more cortical regions and for stimulating at least one cortical region of the one or more cortical regions.

4) At least one electrode set configured for sensing in the DLPFC and for stimulating the DLPFC.

In accordance with some embodiments of the systems, the one or more electrode sets is selected from:

1) At least one electrode set configured for sensing signals associated with neuronal electrical activity in the one or more cortical regions and at least one electrode set configured for stimulating one or more deep brain structures by using temporally interfering (TI) electric fields, and

2) At least one electrode set configured for sensing signals associated with neuronal electrical activity in the one or more cortical regions and for stimulating one or more deep brain structures by using temporally interfering (TI) electric fields.

In accordance with some embodiments of the systems, the one or more electrode sets are selected from, an electrode assembly including two or more electrodes, a multi-electrode array, an implantable electrode array, an injectable mesh electrode array, a multiplexable electrode array, a flexible electrode array, a flexible electrode array adapted to be applied on a cortical surface, a linear electrode array, an Ecog surface electrode array, a μEcog electrode array, an intra-cortically implantable electrode array, a stent electrode, a stent electrode array, neural dust sensing device(s), EEG electrodes, an electrode set including two or more electrodes implanted under the scalp, an electrode set configured for performing non-invasive transcranial frequency interference stimulation (NTIS), an electrode set configured for performing intracranial frequency interference stimulation (ICTIS) and any combinations thereof.

In accordance with some embodiments of the systems, the signals associated with neuronal electrical activity are selected from, extracellularly recorded single neuron action potentials, extracellularly recorded electrical field potentials, and any combinations thereof.

In accordance with some embodiments of the systems, the system also includes a telemetry unit in communication with the at least one processor/controller for wirelessly communicating with an external telemetry unit.

In accordance with some embodiments of the systems, the indication is selected from, a phase alteration of the sensed signals in one or more frequency bands, an alteration in computed spectral power of the sensed signals in the one or more frequency bands, and any combination thereof.

In accordance with some embodiments of the systems, the frequency band is selected from, delta band, theta, mu, alpha, beta, and gamma band, or any combinations thereof.

In accordance with some embodiments of the systems, the at least one processor/controller is selected from, at least one processor/controller external to the cranium of the user, at least one intracranial processor/controller, at least one wearable processor controller, at least one remote processor/controller, at least one digital signal processor (DSP), at least one graphic processing unit (GPU), at least one quantum computing device (QCD), a quantum computer and any combinations thereof.

In accordance with some embodiments of the systems, the indication is selected from, an alteration in a computed weighted phase lag index (wPLI) in the beta frequency band, an alteration in computed spectral power (Pγ) in the gamma frequency band, and an alteration in the computed wPLI in the beta frequency band of cortical electrical activity sensed in one or more electrode pairs at the beta frequency band and an alteration in spectral power at the gamma frequency band.

In accordance with some embodiments of the systems, the at least one power source is selected from, at least one power source external to the cranium of the user, at least one intracranial power source, at least one wearable power source, at least one intracranial power receiver for wirelessly receiving power from an extracranial power source, at least one intracranial power receiver for wirelessly receiving and storing power from an extracranial power source, at least one intracranially implanted induction coil adapted for receiving electrical power from an extracranially disposed induction coil, and any combinations thereof.

There is also provided, in accordance with some embodiments of the methods of the present application, a method for enhancing and/or assisting and/or improving cognitive performance of a user. The method includes the steps of: sensing signals associated with neuronal activity in one or more cortical regions, processing the signals for detecting an indication associated with an intention to perform a cognitive task and/or the presentation of a cognitive task and/or the performing of a cognitive task, and stimulating one or more target brain regions of the user responsive to the detecting of the indication for enhancing and/or improving and/or assisting the cognitive performance of the user.

In accordance with some embodiments of the method, the one or more target brain regions are selected from, one or more deep brain structures, one or more cortical regions, and a combination of one or more deep brain structures and one or more cortical regions.

In accordance with some embodiments of the method, the user is selected from a normal user and a user having a neurological disorder, and/or a psychiatric disorder, and/or a neuro-psychiatric disorder.

In accordance with some embodiments of the method, the user is a user having a neurological disorder, and/or a psychiatric disorder and/or a neuro-psychiatric disorder, and wherein the step of stimulating improves the cognitive performance of the user as compared to the cognitive performance of the user when the step of stimulating is not performed.

In accordance with some embodiments of the method, the neurological disorder and/or the psychiatric disorder and/or the neuro-psychiatric disorder is selected from, ADHD, ADD, OCD, anxiety, depression, a learning deficiency, an attention related deficiency or dysfunction, amnesia, a memory dysfunction, traumatic brain injury, stroke dementia, neurodegenerative disorder, and any combinations thereof.

In accordance with some embodiments of the method, the user is a normal user and wherein the step of stimulating augments the cognitive performance of the user as compared to the cognitive performance of the user when the step of stimulating is not performed.

In accordance with some embodiments of the method, the one or more cortical regions include one or more of prefrontal cortex (PFC), a part of the PFC, a dorsolateral prefrontal cortex (DLPFC), a part of the DLPFC, a temporoparietal cortex (TPC), a part of the TPC, an inferior frontal gyrus (IFG), a part of the IFG, the temporal parietal junction (TPJ), a part of the TPJ, and any combinations thereof.

In accordance with some embodiments of the method, the step of sensing also includes sensing signals associated with neuronal activity in one or more additional cortical regions selected from, a visual cortical region, a region of the primary visual cortex (V1), the medial temporal lobe of the visual cortex, a region of a motor cortex, a region of a pre-motor cortex, a region of a somato-sensory cortex, a region of a auditory cortex, a mesial surface of a right cortical occipital lobe, the associative cortex, other areas of the visual cortex, an auditory cortex, a motor cortex, BA 17, BA 18, BA 19, BA 7, BA 6, BA 5, BA 4 and any combinations thereof, and wherein the step of processing also includes processing the signals sensed in the additional cortical regions to detect the indication associated with an intention to perform a cognitive task and/or the presentation of a cognitive task, and/or performing the cognitive task.

In accordance with some embodiments of the method, the one or more deep brain structures are selected from ventral tegmental area (VTA), striatum, caudate nucleus, putamen, nucleus accumbens (NA), locus ceruleus, hippocampus, amygdala, a deep brain structure of the meso-limbic system, a deep brain structure functionally participating in enhancing and/or facilitating learning, memory and attention focusing, a subcortical region of the brain, a substantia nigra, a dorsal striatum, a part of the limbic structures within a mesocortical system, a part of a nigrostriatal system, a part of tuberoinfundibular system, fornix, nucleus basalis of Meynert (NBM), anterior caudate nucleus, dorsal striatum, anterior thalamic nucleus, central thalamus, lateral hypothalamus, subgenual cingulated region (BA 25), enthorinal cortex, perforant path, medial frontal lobe, subthalamic nucleus and any combinations thereof.

In accordance with some embodiments of the method, the step of stimulating is selected from, stimulating one or more deep brain structures for enhancing cognitive performance of the user, stimulating one or more deep brain structures and one or more cortical regions for enhancing cognitive performance of the user, and stimulating one or more cortical regions for enhancing cognitive performance of the user.

In accordance with some embodiments of the method, the step of stimulating includes stimulating one or more cortical regions selected from a prefrontal cortex (PFC), a part of the PFC, a dorsolateral prefrontal cortex (DLPFC), a part of the DLPFC, a temporoparietal cortex (TPC), a part of the TPC, an inferior frontal gyrus (IFG), a part of the IFG, the temporal parietal junction (TPJ), a part of the TPJ, and any combinations thereof for enhancing and/or augmenting and/or improving cognitive performance of the user.

In accordance with some embodiments of the method, the steps of sensing, processing and stimulating are performed automatically.

In accordance with some embodiments of the method, the performing of one or more steps selected from the steps of sensing, processing and stimulating is user controlled.

In accordance with some embodiments of the method, the method also includes the following steps:

1) stimulating the visual cortex of the user to cause the user to perceive a virtual image of a graphic user interface (GUI).

2) sensing in the motor cortex of the user signals associated with a voluntary intention to perform a movement or the imagining of performing a movement or the performing of a movement.

3) processing the signals sensed in the motor cortex to perform an interaction with the virtual image of the GUI for controlling the performing of one or more steps selected from the steps of sensing, processing and stimulating.

In accordance with some embodiments of the method, the step of processing includes processing the signals using a method selected from, kernel analysis, principal component analysis, spectral analysis methods, common spatial patterns method (CSP), Analytic CSP (ACSP), time domain analytic methods, Frequency Domain analytic methods, supervised pattern classification, cluster seeking methods, likelihood functions and statistical decision.

In accordance with some embodiments of the method, the indication is selected from, a phase alteration of the sensed signals in one or more frequency bands, an alteration in computed spectral power of the sensed signals in the one or more frequency bands, and any combination thereof.

In accordance with some embodiments of the method, the frequency band is selected from, delta band, theta, mu, alpha, beta, and gamma band, or any combinations thereof.

In accordance with some embodiments of the method, the steps of sensing and stimulating are performed in a dorsolateral prefrontal cortex (DLPFC).

In accordance with some embodiments of the method, the step of processing includes Fourier transform (FT) of the sensed signals to obtain power spectra data for multiple electrode pairs, performing phase coupling analysis on the data to compute a weighted phase lag index (wPLI), comparing the computed wPLI to a threshold value and initiating the step of stimulating the one or more target brain regions of the user upon detecting that the computed wPLI is smaller than a threshold value.

In accordance with some embodiments of the method, the step of initiating the step of stimulating includes initiating the step of stimulating after a time delay period starting at the time of the detecting.

In accordance with some embodiments of the method, the step of stimulating includes stopping the sensing for the duration of the step of stimulating.

In accordance with some embodiments of the method, the step of processing includes computing Fourier Transform (FT) of the sensed signals to obtain power spectra data, computing from the power spectra the spectral power in the gamma frequency band (Pγ) value of value of, comparing the computed Pγ to a threshold value and initiating the step of stimulating upon detecting that Pγ is smaller than or equal to a threshold value.

In accordance with some embodiments of the method, the step of initiating the step of stimulating includes initiating the step of stimulating after a time delay period starting at the time of the detecting.

In accordance with some embodiments of the method, the step of stimulating includes stopping the sensing for the duration of the step of stimulating.

In accordance with some embodiments of the method, the indication is selected from, a phase alteration of the sensed signals in one or more frequency bands, an alteration in computed spectral power of the sensed signals in the one or more frequency bands, and any combination thereof.

In accordance with some embodiments of the method, the frequency band is selected from, delta band, theta, mu, alpha, beta, and gamma band, or any combinations thereof.

There is also provided, in accordance with the systems of the present application, a brain computer interface (BCI) system for augmenting and/or assisting and/or improving cognitive performance of a user. The system includes:

1) One or more sensing devices for sensing signals associated with neuronal electrical activity in one or more cortical regions of the user.

2) One or more stimulating devices for providing stimulating signals to one or more target brain regions selected from the group consisting of one or more deep brain structures of the user, one or more cortical regions of the user and a combination of at least one cortical region and at least one deep brain structure of the user.

3) At least one processor/controller in communication with the one or more sensing devices and the one or more stimulating devices, the at least one processor/controller is programmed to process signals sensed in the one or more cortical regions for detecting an indication associated with an intention to perform a cognitive task and/or a presentation of a cognitive task and/or performing of the task, and to control the stimulating of the one or more target brain regions responsive to detecting the indication for augmenting and/or assisting, and/or improving the cognitive performance of the user.

4) At least one power source for energizing the BCI system.

In accordance with some embodiments of the system, the one or more sensing devices include electrodes configured to sense electrical signals associated with electrical activity in the one or more cortical regions.

In accordance with some embodiments of the system, the one or more stimulating devices include electrodes configured to apply electrical stimulating signals to the target brain regions.

Finally, in accordance with some embodiments of the system, at least one sensing device of the one or more sensing devices includes one or more electrode sets configured to sense electrical signals associated with electrical activity in the one or more cortical regions and the at least one stimulating device of the one or more stimulating devices includes one or more electrode sets configured to apply electrical signals to the one or more target brain regions for electrically stimulating the one or more target brain regions.

### BRIEF DESCRIPTION OF THE SEVERAL VIEWS OF THE DRAWINGS

Some embodiments of the invention are herein described, by way of example only, with reference to the accompanying drawings, in which like components are designated by like reference numerals. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1 is a schematic block diagram illustrating the components of a general system for augmenting or enhancing or improving cognitive performance of a user, in accordance with some embodiments of the augmented cognition systems of the present application;

FIG. 2 is a schematic block diagram of a system for augmenting or enhancing or improving cognitive performance of a user, usable for performing general computing tasks, in accordance with an embodiment of the systems of the present application;

FIG. 3 is a schematic block diagram illustrating an embodiment of a system for augmenting or enhancing or improving cognitive performance of a user, including one or more electrode sets for sensing neuronal activity in the dorsolateral prefrontal cortex (DLPFC) and for electrically stimulating several deep brain structures, in accordance with some embodiments of the systems of the present application;

FIG. 4 is a schematic block diagram illustrating a wireless embodiment of a system for augmenting or enhancing or improving cognitive performance of a user including one or more electrode sets for sensing neuronal activity in the dorsolateral prefrontal cortex (DLPFC) and for electrically stimulating one or several deep brain structures associated, inter alia, with learning, memory and regulation of attention, in accordance with some embodiments of the systems of the present application;

FIG. 5 is a schematic block diagram illustrating a system for augmenting or enhancing or improving cognitive performance of a user including several electrode set(s) for sensing neuronal activity in the dorsolateral prefrontal cortex (DLPFC) cortical region and (optionally) in other cortical regions and for electrically stimulating one or several deep brain structures associated, inter alia, with learning, memory and regulation of attention, in accordance with some embodiments of the augmented/enhanced cognition systems of the present application;

FIG. 6 is a schematic diagram illustrating an intracranial system for augmenting or enhancing or improving cognitive performance of a user, disposed within the cranium of the user, in accordance with some embodiments of the systems of the present application;

FIG. 7 is a schematic diagram illustrating a system for augmenting or enhancing or improving cognitive performance of a user, having some system components disposed within the cranium of a user and some other components of the system disposed outside the cranium of the user, in accordance with some embodiments of the systems of the present application;

FIG. 8 is a schematic flow chart illustrating steps of a method for training and/or calibrating a system for augmenting or enhancing or improving cognitive performance of a user, in accordance with some embodiments of the methods of the present application;

FIG. 9 is a schematic flow chart illustrating steps of a method for augmenting or enhancing or improving cognitive performance of a user, in accordance with some embodiments of the methods of the present application;

FIG. 10 is a schematic block diagram illustrating a system for augmenting or enhancing or improving cognitive performance having a single sensing and stimulating electrode set in accordance with some embodiments of the methods of the present application;

FIG. 11 is a schematic block diagram illustrating a system for augmenting or enhancing or improving cognitive performance having sensing and stimulating electrode set(s) for sensing in two cortical regions and for stimulating one or more cortical regions or one or more deep brain structures or a combination of one or more cortical regions and one or more deep brain structures, in accordance with some embodiments of the systems of the present application;

FIG. 12 is a schematic block diagram illustrating a system for augmenting or enhancing or improving cognitive performance, including a set of non invasive electrodes for performing transcranial frequency interference stimulation of deep brain structures and intracranially implanted ECOG electrode arrays for sensing and/or stimulating one or more cortical regions, in accordance with some embodiments of the systems of the present application;

FIG. 13 is a schematic block diagram illustrating the functional components of an intracranial part of the system of FIG. 12;

FIG. 14 is a schematic drawing illustrating a system for augmenting or enhancing or improving cognitive performance, having multiple intracranial ECOG arrays for performing sensing in multiple cortical regions and for performing intracranial frequency interference stimulation of one or more deep brain structures and/or for directly stimulating one or more cortical regions, in accordance with some embodiments of the systems of the present application; FIG. 15 is a schematic functional block diagram illustrating functional components included in the system of FIG. 14; and

FIGS. 16-19 are a schematic flow chart diagrams illustrating the steps of four different exemplary methods for augmenting or enhancing or improving cognitive performance of a user, in accordance with some embodiments of the methods of the present application.

### DETAILED DESCRIPTION OF SPECIFIC EMBODIMENTS OF THE INVENTION

Abbreviations:

The following abbreviations are used throughout the specification and the claims of the present application:

ADD—Attention Deficit Disorder (presently this term has been replaced in the literature by the term “Predominantly inattentive presentation—IA” which is equivalent thereto)

ADHD: Attention Deficit Hyperactivity Disorder.

BA: Brodmann Area

BCI: Brain Computer Interface.

DBS: Deep brain stimulation.

DLPFC: Dorsolaterl prefrontal cortex

DSP: Digital signal processor.

Ecog: Electrocorticogram.

Ecog BCI: Electrocorticographic brain computer interface.

EPROM: Electrically programmable read only memory.

EEPROM: Erasable electrically programmable read only memory.

FMRI: Functional Magnetic resonance imaging.

GUI: Graphic User Interface.

5 HT: 5 hydroxytriptamine.

Hz: Hertz

IC: Integrated Circuit

ICTIS: intracranial temporal interference stimulation

IFG: Inferior frontal gyms

IMU: inertial measurement unit.

KHz: Kilohertz

LAN: Local Area Network

LC: Locus Ceruleus

LFP: Local Field Potential

msec: millisecond

NA: Noradrenaline

NTIS: non-invasive temporal interference stimulation

OCD: Obsessive compulsive disorder.

PFC: Prefrontal cortex.

ROM: Read only Memory.

RAM: Random Access Memory.

SSD: Solid state disk.

TBI: Traumatic Brain Injury

TI: Temporal interference

TPC: Temporoparietal cortex

TPJ: Temporal Parietal junction

VTA: Ventral tegmental area.

VPN: Virtual Private Network.

μV: microvolt

WAN: Wide Area Network

WM: Working Memory.

An aspect of the systems and methods of the present application is that they may be used to perform “cognitive enhancement” in a normal user. Cognition, (and by extension working memory, sustained attention, and other faculties of the DLPFC) may be enhanced by sensing electrical activity in the DLPFC and/or in other cortical regions involved in (such as, for example the TPC, TPJ PFC, and/or any other cortical regions implicated in attention, focusing, sustaining attention, learning and working memory control), detecting in the sensed signals neuronal activity patterns which are associated with learning tasks (such as, for example, associative learning tasks or memorizing tasks, or any other learning tasks requiring attention focusing and increasing attention span), and responsive to such detection, stimulating one or more deep brain structures (and/or some cortical regions) effective in modulating and/or enhancing learning and memory through improving and/or augmenting and/or enhancing the user's attention span, focusing attention on the task and enhancing performance, resulting in cognitive enhancement.

The modulation and/or enhancing or augmenting of the cognitive performance of the user may result from the release of dopamine at synapses of VTA dopaminergic neuronal axons terminating on dendrites or cell bodies within the relevant neural circuits within the DLPFC (or other cortical regions as disclosed hereinabove) which may enhance cognitive performance, inter alia, due to reinforcing of particular relevant cortical circuits involved in the performance of the cognitive task(s), such as, for example, learning and memory.

It is noted that if other deep brain structures are stimulated (independently of or together with the stimulation of the VTA) other types of neuromodulators may possibly be involved in the enhancing or improving or augmenting the cognitive performance of the user, such as, for example 5-hydroxytryptamine (5 HT), and noradrenalin (NA) and/or various different neuropeptides, depending on the specific deep brain structure(s) that are being stimulated.

Another aspect of the systems and methods disclosed herein is to improve cognitive performance in patients or users suffering from neurological or psycho-neurological disorders or impaired cognitive performance (such as, people having brain lesions affecting memory functions, traumatic brain injury (TBI) patients, stroke, dementia, neurodegenerative disorder, attention focusing and learning, whether due to a congenital disorder or due to injury or degeneration of certain brain structures and/or their function (non-limiting examples are patients having ADHD, ADD, OCD, Depression, clinical depression, traumatic brain injury, stroke, amnesia, and more specific types of memory impairment disorders).

In accordance with some embodiments of the systems and methods of the present application, the stimulation of the deep brain structures (such as, but not limited to, the VTA, the striatum, the caudate nucleus, the putamen, the nucleus accumbens, the locus ceruleus, the hippocampus, the amygdale, and/or any other deep brain structure of the meso-limbic system, and/or any other deep brain structure functionally participating in enhancing or facilitating learning, memory and attention focusing and other types of user's cognitive performance) in normal users or in patients having the disorders disclosed hereinabove (or any other neurological or psychiatric and/or neuro-psychiatric disorder or deficiency) may be performed fully automatically upon detection of certain specific patterns of neuronal activity in the DFPLC and/or in some other cortical regions (such as, for example the TPC, TPJ PFC, and/or any other cortical regions implicated in attention focusing, sustaining attention, learning and working memory control). This stems from the need for precise timing of the stimulation with respect to the timing of task presentation as shown by Husam A. Katnani, et al. in the article entitled “Temporally Coordinated Deep Brain Stimulation in the Dorsal and Ventral Striatum Synergistically Enhances Associative Learning.” published in Scientific Reports 6, Nature, Article number: 18806 (2016). It is noted however, that in the experiments described in the paper the task presentation and therefore the deep brain stimulation were not triggered by or linked to any activity sensed or recorded in the monkey's brain.

In some embodiments of the systems of the present application, the system may operate in a “Task untethered” mode, meaning that the user is not performing a specific task, but going about his/hers day normally. When the system detects that WM or Attention circuits are engaged in the DLPFC (or in other cortical regions), the system automatically delivers the stimulation to deep brain structures to achieve reinforcement of cognitive performance.

In some other embodiments of the systems of the present application, the system may operate in a “Task dependent” mode in which the system stimulates deep brain structures only during the course of a clinically administered, or self-administered task, such as, for example, an A-not-B type of task.

In some other embodiments of the systems of the present application operating automatically and autonomously, the user may have no control over the timing and spatio-temporal pattern of the delivery of stimulation to deep brain structures, which are automatically generated and precisely timed by the processor/controller(s) controlling the stimulating electrode set(s) of the system, in order to optimize the enhancing effect of the stimulation on the users performance in such learning, and/or memorizing tasks, or any other task requiring augmented and focused attention and/or motivation.

However, in some embodiments of the systems disclosed herein, the user and/or patient may have the ability to voluntarily switch on or off the mode of operation of the system. For example, the user or patient may be able to voluntarily activate or deactivate the “cognitive performance enhancing” operation of the system by either switching on or off the stimulation of deep brain structures by voluntarily controlling the operation of the stimulation of the deep brain structure(s) (by disabling stimulation or enabling stimulation). Such controlling methods are disclosed in more detail hereinafter with respect to specific embodiments of the systems.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. It is expected that during the life of a patent maturing from this application many relevant devices, systems and methods for sensing neuronal electrical activity (either of single neurons and/or of neuronal ensembles) and for stimulation of single or multiple neurons will be developed and the scope of the term “sensing electrode set”, “sensing electrode set(s)” “Stimulating electrode set” and Stimulating electrode set(s)” are intended to include all such new sensing and stimulating technologies, respectively a priori.

Similarly, it is expected that during the life of a patent maturing from this application many relevant devices, systems and methods for sensing signals associated with neuronal electrical activity (either of single neurons and/or of neuronal ensembles) and for stimulating of single or multiple neurons will be developed and the scope of the terms “sensing” and “recording” and “stimulating” are intended to include all such new technologies a priori.

As used herein the term “about” refers to ±10%. The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “normal user” and “normal person” and all their plural forms are interchangeably used throughout the specification and the claims of the present application to denote a person or user that does not suffer from a neurological and/or psychological and/or neuropsychological disorder that impairs one or more aspects of cognitive performance. It is noted that such a normal user or person may be suffering from any other illnesses or disability conditions that are not directly related to cognitive impairment.

The term “electrode set” and all of its plural forms are used throughout the specification and the claims of the present application to denote any electrode arrangement including two or more electrodes configured for sensing electrical activity in one or more brain regions and/or for stimulating one or more brain regions, and/or for both sensing in and stimulating of one or more brain regions. It is noted that these terms may refer to just the electrodes but may also refer to any electronic and/or electrical circuits that are either included as part of the structure of the electrodes or as parts of an electrode assembly or electrode array and used for signal amplification, signal conditioning, signal filtering close to the electrode sensing part(s). For example, if an Ecog type electrode array includes electrical and/or electronic components integrated into the sensing electrodes or in the vicinity of the electrodes on the substrate by which the array is supported, the entire array and electronic/electrical components associated therewith may be referred to as “an electrode set”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

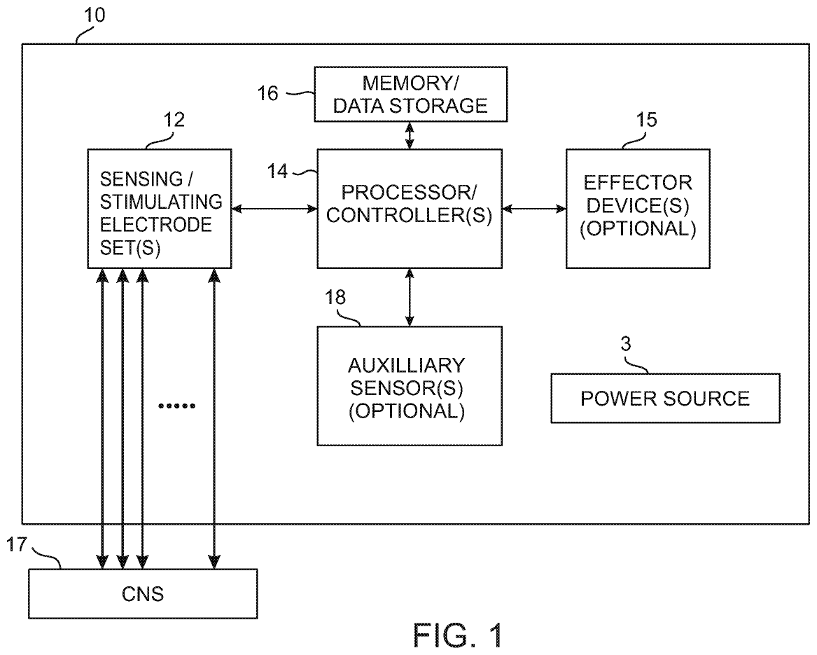

Reference is now made to FIG. 1 which is a schematic block diagram illustrating the components of a general system for augmenting or enhancing or improving cognitive performance of a user, in accordance with some embodiments of the augmented cognition systems of the present application.

The system 10 includes one or more sensing/stimulating electrode set(s) 12, which are in communication with one or more processor/controller(s) 14. The processor/controller(s) 14 may be suitably connected to one or more memory and/or data storage devices 16 for storing and retrieving data as is known in the art.

The processor/controller(s) 14 may be one or more computing devices selected from, one or more processor/controller external to the cranium of the user, one or more intracranial processor/controller, at least one wearable processor/controller, at least one remote processor/controller, at least one digital signal processor (DSP), at least one graphic processing unit (GPU), at least one quantum computing device processing unit (CPU), or any combinations of the above. In some embodiments, the processor/controller(s) 14 may include and/or emulate a neural network. For example, the processor/controller(s) 14 may include or may be connected to one or more neuromorphic ICs (which may be also included in any of the cognition augmenting/enhancing systems of the present application. Alternatively and/or additionally, the processor/controller 14 may be programmed to emulate one or more neural networks by software operative on the processor/controller(s) 14.

Furthermore, the processor/controller 14 may have access to the “cloud” via the internet (preferably, wirelessly, but also possibly in a wired way) or through any other type of network, such as, for example, a LAN, a WAN, a VPN or any other type of wired or wirelessly accessible network.

In some embodiments, the processor/controller(s) 14 may include wireless communication circuits, such as Bluetooth, or WiFi communication units or circuits (not shown in detail any of the figures for the sake of clarity of illustration). Such wireless communication means may enable the processor/controller to wirelessly communicate with external devices, such as for example, a remote computer, a server, a cellular telephone, or any other type of. Such embodiments may be useful in cases in which the processing power of the processor/controller(s) 14 is limited. Such embodiments may allow the offloading of some or all of the computational burden to other processing devices, such as remote computer(s), servers, a cluster of computers or any other suitable computing devices, and may enable the use of cloud computing, or parallel computing for processing the data recorded/sensed in the DLPFC or other brain regions reducing the computational load on the processor/controller(s) 14. The results of such offloaded computations may then be returned or communicated (preferably wirelessly) to the processor/controller 14 and used for performing the controlling of the stimulation of the appropriate deep brain structures as disclosed herein.

Preferably, for invasive systems, the processor/controller(s) 14 are microminiaturized to have the smallest possible size and to minimize power requirements and heat output. However, if wearable computing devices or similar external computers devices are being used, the size and power requirement of the computing devices may be increased.

The processor/controller(s) 14 and/or the one or more sensing/stimulating electrode set(s) 12 may include any necessary electrical circuitry (not shown for the sake of clarity of illustration) required for conditioning, and/or amplifying, and/or filtering and/or digitizing the electrical signals sensed by the one or more sensing/stimulating electrode set(s) 12 (such as, for example, an analog to digital converter (ADC), signal amplifiers, analog filters, digital filters or any other suitable electrical and/or electronic or opto-electronic circuitry) as is known in the art of bio-signal processing.

The processor/controller(s) 14 and/or the one or more sensing/stimulating electrode set(s) 12 may also include any electrical circuitry (not shown for the sake of clarity of illustration) for providing electrical stimulation to nervous tissues through the one or more sensing/stimulating electrode set(s) 12 as is known in the art. Such electrical circuitry may include, a suitable (optional) power source, such as one or more current sources, multiplexing circuitry, one or more electrical pulse generators, timing circuitry and any other electrical circuitry necessary for stimulating neurons through one or more of the sensing/stimulating electrode set(s) 12, as is well known in the art.

In all system embodiments disclosed herein and illustrated in the drawing figures in which the processor/controller(s) 14 is shown to be directly connected to one or more stimulating electrode sets (or sensing and stimulating electrode set(s)) without explicitly showing such stimulating circuitry it is to be understood that such circuitry (such as, for example, one or more current sources, multiplexing circuitry, one or more electrical pulse generators, timing circuitry and any other electrical circuitry necessary for stimulating neurons through one or more of the sensing/stimulating electrode sets) may be included in the processor/controller(s) 14 and is not shown in detail for the sake of clarity of illustration.

However, the system 10 (or any of the other systems disclosed in the present application and illustrated in the drawing figures) may include a suitable power source 3 included in the system for providing power to any power requiring system components). It is noted that the power lines connecting the power source 3 to any power requiring components are not shown in any of FIGS. 1-5 hereinafter, for the sake of clarity of illustration.

The system 10 may also (optionally) include one or more auxiliary sensors 18 suitably connected and coupled to the processor/controller(s) 14. The optional auxiliary sensors 18 may include one or more sensors selected from an imaging sensor, a monochrome imaging sensor, a color imaging sensor, an infrared (IR) imaging sensor, an ultraviolet (UV) imaging sensor, an ionizing radiation sensor, a Geiger counter, a microphone, a stereoscopic depth sensor, an inertial measurement unit (IMU), one or more accelerometers, a vibrometer, a temperature sensor, a microphone, an acoustic sensor for sensing sound and/or infrasound and/or ultrasound, a thermistor a sensor for sensing and/or detecting volatile compounds in air, and any combinations thereof.

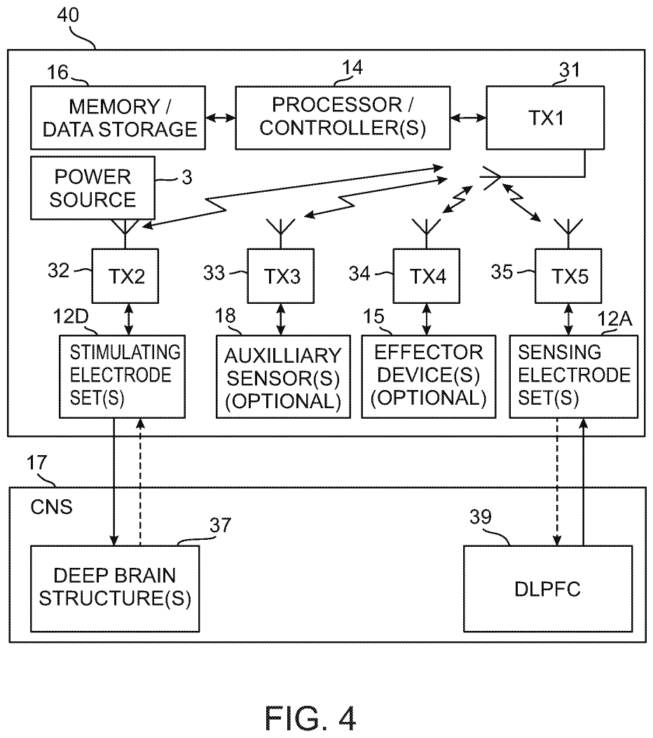

Generally, any suitable sensors known in the art including but not limited to the above described sensors that may be attached to or born by or worn by the user of the system 10 may be included in the auxiliary sensors 18. Additionally, the auxiliary sensors 18 may also include any sensors which are too heavy or too large or cumbersome to be worn or attached or worn by the user of the system 10, by having such auxiliary sensors wirelessly communicate with the processor/controller(s) 14 (suitable wireless communication systems may be used as is known in the art). Such suitable wireless communication devices are not shown in FIG. 1 for the sake of clarity of illustration but may be similar to the transceivers TX1-TX4 of FIG. 4. Such auxiliary sensors may include, for example, radar based sensor devices, LIDAR devices, geophones, sonar devices or any other large sensors or sensor systems known in the art.

The auxiliary sensors 18 may include sensors (such as, for example, a camera or stereoscopic depth sensor, or laser range finder) that may provide the processor/controller(s) 14 with sensed data usable for providing the user with geo-contextual information or data, (such as the position of various real objects or of the user's body or body parts in space and/or relative to other objects in the environment).

The system 10 may also (optionally) include one or more effector devices 15. The effector devices 15 may be effector devices implanted within the body of the user, but may also be external effector devices carried by the user and/or externally attached to the body of the user or to one or more garment worn by the user on the body. The effector devices 15 may also be any type of external effector device placed anywhere and remotely and wirelessly controllable and/or wirelessly operable by the user that is using the system 10. The effector device(s) 15 may be effector device attached to or carried by the user, an effector device carrying the user, a prosthesis, a motorized vehicle, a land vehicle, an airborne vehicle, a marine vehicle, an effector device in the vicinity of the user, a remote effector device, a drone, a motorized exoskeleton device carrying the user, a robotic device operable by the user, a sound source, an ultrasound source, an audio speaker, a visible light source, an IR light.

The effector device(s) 15 may also include any non-mutually exclusive combinations of the above described effectors and any combinations thereof.

In accordance with some embodiments, the effector device(s) maybe be selected from, a device for controllably delivering a substance or a composition to the body of the user or to a selected part of the body, a device for medically and/or therapeutically treating the body of the user and/or any combinations thereof. The substance or composition may be selected from, a drug, a therapeutic agent, a stimulant, a sedative, an anti-inflammatory agent, a muscle relaxing agent, an antibacterial agent, an antifungal agent, an antiviral agent, a nutrient, a hormone, a neurotransmitter, a neuro-protective agent, a vitamin, an anticoagulant agent, or and any non-mutually exclusive or medically contraindicated combinations of the above described substances.

Some of the effector devices may be therapeutic devices for medically and/or therapeutically treating the body of the user and may be selected from, a device for delivering electrical stimulation to said body or to a part thereof, a device for heating or cooling said body or a selected region or organ thereof, a device for delivering therapeutic electromagnetic radiation to said body or to a part thereof, and any combinations thereof.

The memory/data storage device(s) 16 may be any type of memory and/or data storage device(s) known in the art for storing and/or retrieving data. Non limiting, exemplary memory and/or data storage devices usable in the system 10 (and in any of the other cognition augmenting/enhancing systems disclosed hereinafter), may include one or more devices such as ROM, RAM, EPROM, EEPROM, Flash memory devices of any type known in the art, optical memory and/or storage devices and any combinations thereof.

The sensing/stimulating electrode set(s) 12 may be any type of sensing and/or stimulating electrodes known in the art which are capable of interfacing with one or more than one parts of the nervous system 17 of a user, such as, one or more parts of the central nervous system of the user, but also any other part or parts of the nervous system of the user including but not limited to, any cortical regions, one or more limbic structures, the sympathetic system, the parasympathetic system, the spinal cord, the peripheral sensory system, the retina and/or optical nerve and any other nervous tissues in the body of the user.

The sensing/stimulating electrode set(s) 12 may be implemented as different types of electrodes set(s) or electrode group(s), depending on the region of the nervous system that they interact with. Such different electrode set(s) are well known in the art and several forms of such Electrode set(s) are commercially available on the market. The structure and operation of such electrode set(s) is well known in the art, and is therefore not described in detail hereinafter. Briefly, the Electrode set(s) 12 may be selected from, a single electrode set, a Multi-electrode sets, an electrode array, a stent type electrode array for insertion into a blood vessel within the brain, a flexible single surface electrode, a flexible multi electrode array for recording from and/or stimulation of one or more surfaces of the brain/or CNS, including but not limited to cortical regions and/or other brain surface regions for recording and/or stimulation thereof, flexible mesh-type electrode arrays for internal implantation within cortical regions and/or cortical layers, flexible mesh-type electrode arrays for internal implantation within any deep brain structures, flexible mesh type electrode arrays that may be placed on the cortical surface, retinal electrode sets for implantation within the eye and any combinations of the above electrode and electrode set(s) types.

The methods for construction and for of use of such diverse types of electrode types and their associated electronic circuits, usable in the enhanced/augmented/improved cognition systems, as well as methods and algorithms for processing sensed neuronal activity to generate commands for controlling effector devices (including prosthetic limbs) or to perform various computations (both analog and/or digital) for pattern recognition and/or pattern detection and/or pattern classifications, and/or to perform other general computational tasks are well known in the art and are described in detail, inter alia, in some of the following references:

1. Jeneva A. Cronin, Jing Wu, Kelly L. Collins, Devapratim Sarma, Rajesh P. N. Rao, Jeffrey G. Ojemann & Jared D. Olson. “Task-Specific Somatosensory Feedback via Cortical Stimulation in Humans.”, IEEE Transactions on Haptics, DRAFT. DOI: 10.1109/TOH.2016.2591952.

2. Kay Palopoli-Trojani, Virginia Woods, Chia-Han Chiang, Michael Trumpis & Jonathan Viventi. “In vitro Assessment of Long-Term Reliability of Low-Cost μECoG Arrays.”, Micro Electro Mechanical Systems, 2016, IEEE International Conference, 24-28 Jan. 2016, DOI: 10.1109/MEMSYS.2016.7421580.

3. Shota Yamagiwa, Makoto Ishida & Takeshi Kawano. “SELF-CURLING AND—STICKING FLEXIBLE SUBSTRATE FOR ECoG ELECTRODE ARRAY”, Micro Electro Mechanical Systems, 2013, IEEE 26^{th} International Conference, 20-24 Jan. 2013. DOI: 10.1109/MEMSYS.2013.647428.

4. Yusuke Morikawa, Shota Yamagiwa, Hirohito Sawahata, Makoto Ishida & Takeshi Kawano. “AN ORIGAMI-INSPIRED ULTRASTRETCHABLE BIOPROBE FILM DEVICE”, MEMS 2016, Shanghai, CHINA, 24-28 Jan. 2016, 978-1-5090-1973-1/16/$31.00 ©2016 IEEE, PP. 149-152.

5. Nikita Pak, Joshua H. Siegle, Justin P. Kinney, Daniel J. Denman, Tim Blanche & Ed S. Boyden. Closed-loop, ultraprecise, automated craniotomies. Journal of Neurophysiology 113, April 2015, Pp. 3943-3953.

6. Tian-Ming Fu, Guosong Hong, Tao Zhou, Thomas G Schuhmann, Robert D Viveros & Charles M Lieber., “Stable long-term chronic brain mapping at the single-neuron level.”, Nature Methods, Vol. 13, No. 10, October 2016, Pp. 875-882.

7. Chong Xie, Jia Liu, Tian-Ming Fu, Xiaochuan Dai, Wei Zhou & Charles M. Lieber, “Three-dimensional macroporous nanoelectronic networks as minimally invasive brain probes.”, Nature Materials, Vol. 14, December 2015, Pp. 1286-1292.

8. Guosong Hong, Tian-Ming Fu, Tao Zhou, Thomas G. Schuhmann, Jinlin Huang, & Charles M. Lieber. “Syringe Injectable Electronics: Precise Targeted Delivery with Quantitative Input/Output Connectivity”, Nano Letters, Vol. 15, August 2015, Pp. 6979-6984. DOI: 10.1021/acs.nanolett.5b02987.

9. Jia Liu, Tian-Ming Fu, Zengguang Cheng, Guosong Hong, Tao Zhou, Lihua Jin, Madhavi Duvvuri, Zhe Jiang, Peter Kruskal, Chong Xie, Zhigang Suo, Ying Fang & Charles M. Lieber. “Syringe-injectable electronics.”, Nature Nanotechnology, Vol. 10, July 2015, Pp. 629-636. DOI: 10.1038/NNANO.2015.115.

10. David T. Bundy, Mrinal Pahwa, Nicholas Szrama & Eric C. Leuthardt. Decoding three-dimensional reaching movements using electrocorticographic signals in humans”, Journal of Neural Engineering, Vol. 13, No. 2, 2016, Pp. 1-18. DOI:10.1088/1741-2560/13/2/026021.

11. Takufumi Yanagisawa, Masayuki Hirata, Youichi Saitoh, Haruhiko Kishima, Kojiro Matsushita, Tetsu Goto, Ryohei Fukuma, Hiroshi Yokoi, Yukiyasu Kamitani & Toshiki Yoshimine, “Electrocorticographic Control of a Prosthetic Arm in Paralyzed Patients.”, Annals of Neurology, Vol. 71, No. 3, March 2012, Pp. 353-361. DOI: 10.1002/ana.22613.

12. Wei Wang, Jennifer L. Collinger, Alan D. Degenhart, Elizabeth C. Tyler-Kabara, Andrew B. Schwartz, Daniel W. Moran, Douglas J. Weber, Brian Wodlinger, Ramana K. Vinjamuri, Robin C. Ashmore, John W. Kelly & Michael L. Boninger. “An Electrocorticographic Brain Interface in an Individual with Tetraplegia”, Plos One, Vol. 8, No. 2, February 2013, Pp. 1-8. DOI:10.1371/journal.pone.0055344.

13. Kay Palopoli-Trojani, Virginia Woods, Chia-Han Chiang, Michael Trumpis & Jonathan Viventi. “In vitro assessment of long-term reliability of low-cost μECoG arrays.”, Engineering in Medicine and Biology Society, 38th Annual International Conference of the IEEE, 16-20 Aug. 2016.

14. L. Muller, S. Felix, K. Shah, K. Lee, S. Pannu & E. Chang. “Thin-Film, Ultra High-Density Microelectrocorticographic Decoding of Speech Sounds in Human Superior Temporal Gyrus.”, Lawrence Livermore National Laboratory, IEEE Engineering in Medicine and Biology Conference, Orlanda, Fla., United States, Aug. 16, 2016 through Aug. 20, 2016. LLNL-CONF-684084.

15. Jonathan Viventi, et al., “Flexible, Foldable, Actively Multiplexed, High-Density Electrode Array for Mapping Brain Activity in vivo.”, Nature Neuroscience, Vol. 14, No. 12, Pp. 1599-1605. DOI:10.1038/nn.2973.

16. Thomas J. Oxley et al. Minimally invasive endovascular stent-electrode array for high-fidelity, chronic recordings of cortical neural activity. Nature Biotechnology, Vol. 34, No. 3, February 2016. DOI:10.1038/nbt.3428.

17. Edward S. Boyden, Feng Zhang, Ernst Bamberg, Georg Nagel & Karl Deisseroth, “Millisecond-timescale, genetically targeted optical control of neural activity”, Nature Neuroscience, Vol. 8, No. 9, September 2005, Pp. 1263-1268. DOI:10.1038/nn1525.

18. Karl Deisseroth. “Optogenetics”, Nature Methods, Vol. 8, No. 1, January 2011, Pp. 26-29. DOI: 10.1038/NMETH.F.324.

19. Karl Deisseroth. “Optogenetics: 10 years of microbial opsins in neuroscience. ”Nature Neuroscience, Vol. 18, No. 9, September 2015, Pp. 1213-1225.

20. Andre Berndt Karl Deisseroth.” Expanding the optogenetics toolkit: A naturally occurring channel for inhibitory optogenetics is discovered.”Science, Vol. 349, No. 6248, Aug. 7, 2015, Pp. 590-591.

21. S. Yamagiwa, M. Ishida & T. Kawano. “Flexible parylene-film optical waveguide arrays.”, Applied Physics Letters, Vol. 107, No. 083502, 2015, Pp. 1-5. DOI: 10.1063/1.4929402.

22. Michael Joshua Frank, Johan Samanta, Ahmed A. Moustafa & Scott J. Sherman. “Hold Your Horses: Impulsivity, Deep Brain Stimulation, and Medication in Parkinsonism.”, Science, Vol 318, No. 5854, December 2007, Pp. 1309-1312. DOI: 10.1126/science.1146157.

23. David J. Foster & Matthew A. Wilson. “Reverse replay of behavioural sequences in hippocampal place cells during the awake state.”, Nature 04587, Pp. 1-4. DOI:10.1038.

24. Nir Grossman, David Bono, Nina Dedic, Suhasa B. Kodandaramalah, Andrii Rudenko, Ho-Jun Suk, Antonino M. Cassara, Esra Neufeld, Niels, Li Huei Tsai, Alvaro Pascual-Leone and Edwards S. Boyden, “Non-Invasive Deep Brain Stimulation via Temporally Interfering Electric Fields”, Cell 169, pp 1029-1041, June 1, 2017.

25. U.S. Pat. No. 8,121,694 to Molnar et al. entitled “Therapy control based on a patient movement state”.

The type of electrical activity which may be sensed/recorded by the sensing/stimulating electrode set(s) 12 may include single neuron electrical activity (extracellularly recorded single neuronal action potentials), simultaneously sensed/recorded electrical activity of several neurons (extracellularly recorded multiple neuronal action potentials), sensed extracellularly recorded field potentials, Electrocorticogram type sensing/recording (Ecog) of summed electrical activity from multiple neurons (such as, Ecog recorded with surface recording Ecog array types)

Additionally, while electrode sets including electrically conducting electrodes for recording neuronal electrical activities representative of single or multiple neuronal electrical activities and for electrically stimulating single or multiple neurons are preferred due to their well characterized properties and interactions with neuronal tissues, the systems of the present application are not limited to electrically recording and stimulation types of devices using such electrode sets. Rather, other types of sensing and/or stimulating devices may also be used to replace the electrode set(s) 12 of the system 10. For example, sensing and/or stimulating devices using optical detection of neuronal tissue activity may be also used and possibly stimulating devices using optical methods for stimulating single or multiple neurons may also be used. Such optical devices are disclosed for example in the following references:

1. Edward S. Boyden, Feng Zhang, Ernst Bamberg, Georg Nagel & Karl Deisseroth. “Millisecond-timescale, genetically targeted optical control of neural activity.”, Nature Neuroscience, Vol. 8, No. 9, September 2005, Pp. 1263-1268. DOI:10.1038/nn1525.

2. Karl Deisseroth. “Optogenetics.”, Nature Methods, Vol. 8, No. 1, January 2011, Pp. 26-29. DOI: 10.1038/NMETH.F.324.

3. Karl Deisseroth. “Optogenetics: 10 years of microbial opsins in neuroscience. “Nature Neuroscience, Vol. 18, No. 9, September 2015, Pp. 1213-1225.

4. Andre Berndt, and Karl Deisseroth.” Expanding the optogenetics toolkit: A naturally occurring channel for inhibitory optogenetics is discovered.”Science, Vol. 349, No. 6248, Aug. 7, 2015, Pp. 590-591.

Other types of electrode sets that may be usable in the systems of the present application may include any type of electrode sets(s) disclosed in any references disclosed in the present application.

For example theoretical calculations indicate that certain types of “neural dust” implementations using ultrasonic communication methods may enable very small (about 50 micron sized) non-tethered wireless devices to be implanted in neuronal tissues for sensing and/or stimulation purposes. Examples of such neural dust implementations may be found in the following publications:

1) Dongjin Seo, Ryan M. Neely, Konlin Shen, Utkarsh Singhal, Elad Alon, Jan M. Rabaey, Jose M. Carmena and Michel M. Maharbiz, entiteled “Wireless Recording in the Peripheral Nervous System with Ultrasonic Neural Dust”, published in Neuron 91, 529-539, Aug. 3, 2016.

2) Biederman William et al. “A Fully Integrated Miniaturized (0.125mm²) 10.5 μW wireless neural sensor”. Published in IEEE Journal of solid State Circuits, Vol. 48 Issue 4, April 2013: DOI: 10.11o9/JSSC 2013.2238994.

Ecog electrode arrays, methods for their use and methods and algorithms for analyzing neuronal activity related signals sensed thereby are disclosed, among others, in the following publications:

1) David T Bundy, Mrinal Pahwa, Nicolas Szrama and Eric C Leuthardt,” decoding three-dimensional reaching movements using electrocorticographic signals in humans”, J. Neural Eng. 13, 23 Feb. 2016.

2) Gerwin Schalk and Eric C Leuthardt, “Brain—Computer Interfaces Using Electrocorticographic signals”, IEEE Reviews In Medical Engineering, Vol. 4, 2011.

3) Eric C Leuthardt, Gerwin Schalk, Jonathan R Wolpaw, Jefrey G Ojemann and Daniel W Moran; “A Brain-Computer Interface Using Electrocorticographic Signals In Humans”. J. Neural Eng. 1. Pp. 63-71 (2004).

The sensing/stimulating electrode set(s) 12 may be any combination of one or more electrodes or electrode sets of several types. For example, for cortical region sensing/stimulation, the electrode set(s) 12 may include surface recording semi-invasive electrodes with single or multiple electrodes placed on the surface of the brain, invasive electrode set(s), such as one or several Utah arrays or other multi-electrode array types that are invasively implanted within the relevant cortical layers by penetrating the cortical surface. Invasively implanted Ecog type electrode arrays disposed on a cortical surface or on the surface of the Dura. In some embodiments (typically, in applications requiring non-invasive sensing/stimulation), the Electrode set(s) 12 may also include extra-cranial EEG type electrodes placed on the surface of the scalp of the user as is known in the art.

For applications requiring sensing and/or stimulation of deep brain regions or deep brain structures, the electrode set(s) 12 may include one or more invasive types of electrodes or electrode arrays that may be stereotactically implanted within one or more deep brain structures. Such electrode set(s) may also include deeply injectable flexible electrode arrays (of the mesh type or of any other type, which may be implanted by injecting them into the deep brain structure(s). Additionally, stent type device(s) including sensing and/or stimulating electrodes or electrode arrays may be semi-invasively (or minimally invasively) delivered and disposed within a blood vessel in the vicinity of such deep brain structure(s) to perform sensing and or stimulating within such deep brain structure(s), as disclosed in the article by Oxley et al., “Minimally invasive endovascular stent-electrode array for high-fidelity, chronic recordings of cortical neural activity”, in Nature Biotechnology 34(3), February 2016 DOI: 10.1038/nbt.3428.

The electrodes set(s) 12 of the system 10 are arranged to sense the neuronal activity within various different regions of the brain and to stimulate one or more regions of the central nervous system 17 of the user to evoke neuronal activity in the stimulated CNS region(s). The various techniques and methods for placement of such electrode set(s) on the scalp and/or for implantation of surface cortical electrode set(s), electrode arrays and or implantation of penetrating electrodes within the cortex or in deep brain structure is well known in the art, are not the subject matter of the present application and is well disclosed in the literature as well as in the references cited herein.

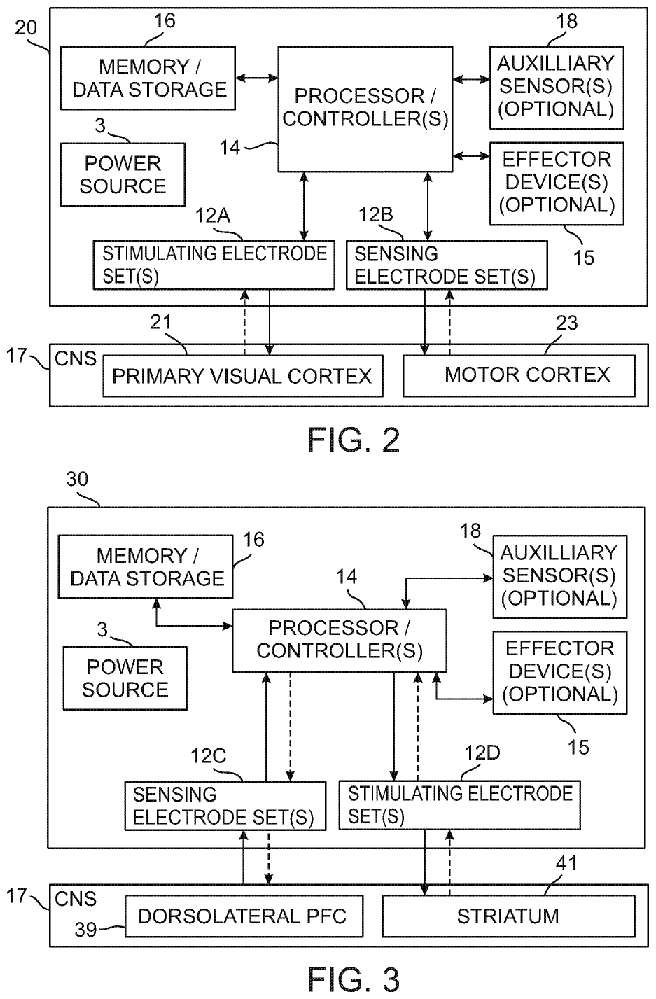

Reference is now made to FIG. 2, which is a schematic block diagram of a system for augmenting or enhancing or improving cognitive performance of a user, usable for performing general computing tasks, in accordance with an embodiment of the systems of the present application. The system 20 includes the processor controller(s) 14 as disclosed in detail hereinabove. The system 20 may also include the memory/data storage device(s) 16, the (optional) auxiliary sensor(s) 18 and the (optional) effector device(s) 15 suitably coupled to the processor/controller(s) 14 as disclosed in detail hereinabove. The system 20 may also include a sensing electrode set(s) 12B for sensing (and/or recording) neuronal activity in the motor cortex 23 (and/or optionally in the premotor cortex) and another sensing electrode set(s) 12B for stimulating the primary visual cortex 21 for causing the user of the system 20 to perceive a virtual image within the field of view of the user as a result of the stimulation of the visual cortex.

The virtual image may be integrated with or superimposed over the “real” visual image of the environment as received by the eyes of the user and relayed normally through the visual pathway to the visual cortex.

The virtual image perceived by the user of the system 20 may be any desired image useful to the user for performing various tasks and/or for presenting data or information to the user (such as internal bodily information or provided by medical sensors included the auxiliary sensor(s) 18).

The information or data presented to the user may be graphic information (an image or images), and/or alphanumeric (such as textual information including characters and/or numbers) and any suitable combination of such visually perceptible images. For example, by stimulating the primary visual cortex 21 (or any other part or region of the visual cortex) a virtual image may be perceived by the user, which may include a virtual graphic user interface (GUI) which may enable the user to perform one or more general computing task. Such general computing tasks may include but are not limited to, operating and/or controlling the operation of any software program(s) (or any subroutine thereof) which is operable on the processor/controller(s) 14.

For example, the stimulating of the primary visual cortex 21 may cause the user to perceive a virtual dialog box superimposed upon the normally perceived field of view (FOV) visibly observed by the user. Such virtual dialog box may include selectable options that may be selected or chosen by “pointing at” or “clicking” on “virtual buttons” included in the virtual dialog box by, for example moving a virtual cursor over to the virtual button. Furthermore, the user may also interact with the artificially induced virtual image or dialog box, etc. by translating, rotating or scaling 3D Tools or 2D or 3D content using natural gestures such as grasping, pinching or grabbing the content with one or two closed or semi closed “grab” or pinch” gestures and manipulating the content by moving (or by planning and/or intending to move) the center of the hand around the user's space. Additional embodiments include the ability to move more virtual limbs by moving or planning to move one physical one. In traditional virtual reality (VR) devices and systems, such interactive images for controlling computing tasks are typically presented to the user by a HUD device or by virtual reality goggles or eyeglasses and are projected into the retina of the user to be conveyed normally through the visual pathway of the user to be perceived by the user. However, in contrast, the virtual image(s) of the present application are a result of direct stimulation of the visual cortex (primary visual cortex and/or any other region(s) of the visual cortex or any desired combination of such regions of the visual cortex).

Such presentation of images acquired by using an external imager images by direct stimulation of the visual cortex is known in the art and has been successfully used for providing blind patients with an image related to the environment as sensed by a video camera by stimulation of the visual cortex of the patient. However, in the system 20 the user may interact with the virtual image (such as the virtual dialog box, a cursor image, or any other graphic image or symbol) by using the system (such as, but not limited to, the sensing electrode set(s) 12B) to sense neuronal activity in the motor cortex 23 resulting from the user voluntarily moving an arm or even actively planning or intending to move an arm (without actually moving the arm) in a certain direction.

It is noted that the use of BCIs to sense neuronal activity in the motor cortex to control the movement of a prosthesis is well known in the art and may be performed by suitable processing of the signals sensed in the motor cortex to generated commands for operating the prosthesis as is disclosed in detail by David T. Bundy, Mrinal Pahwa, Nicholas Szrama & Eric C. Leuthardt, in the paper entitled, “Decoding three-dimensional reaching movements using electrocorticographic signals in humans.”, published in Journal of Neural Engineering, Vol. 13, No. 2, 2016, Pp. 1-18. DOI:10.1088/1741-2560/13/2/026021.

To the best knowledge of the inventor of the present invention, using the sensing and processing of neuronal activity in the motor cortex to interact with a virtual image presented to the user by direct stimulation of the visual cortex for the purpose of performing a general computing task has never been taught or even suggested.

The general computing tasks may be, for example, initiating, or starting or stopping the execution of a computer program programmed into the processor/controller(s) 14, interacting with a virtual graphic user interface of such a program (presented by stimulation of the visual cortex by the stimulating electrode set(s) 12A under control of the processor/controller(s) 14), displaying data and/or information to the user, interacting with a virtual GUI for controlling the operation of one or more of the effector device(s) 15 through a computer software residing in the processor/controller 14, or any other type of computing task performable by such voluntary active interaction (through sensing in the motor cortex and processing the sensed signals to control the interaction of the user with a virtual image perceived as a result of stimulation of the visual cortex of the user controlled by the processor/controller(s) 14.

One of the advantages of the system 20 is that it eliminates the need for an HUD or VR goggles or other VR devices, since the virtual image is perceived by the user as a result of directly stimulating the visual cortex.

Another advantage is that, in contrast to performing real limb movements to interact with an image, recording in the premotor cortex may be faster as it may precede the activity in the motor cortex and musculo-skeletal system activation by a substantial amount of time (typically by about 200-500 microseconds). Thus, the system 20 may advantageously react faster than other systems using VR equipment to performing tasks, which may improve the user reaction time in certain tasks. For example, this may be highly advantageous for improving the speed of operating and/or controlling of certain types of the effector device(s) 15. In such tasks as, for example, operation of an airborne vehicle, or a land vehicle, where reaction time of the user may be very important, there is a clear advantage to the cognitive enhancing systems disclosed herein. Another advantage may be the ability to control several virtual limbs from the movement planning, or direct movement of a single limb. This one-to-many approach may allow users finer or more multi-dimensional control, in a very intuitive manner.

The systems of the present application may also be used for augmenting and/or improving and/or enhancing and/or controlling and/or modulating the performance of cognitive tasks (such as, for example, attention focusing, attention level, short term memory performance, long term memory performance, working memory performance, in normal users or in certain patients having certain disorders, as disclosed hereinafter,

The systems of the present application may also be used for augmenting and/or improving and/or enhancing and/or modulating the performance of cognitive tasks such as, for example, increasing the number of working memory storage items for different kinds of stimuli, Increasing the amount of time a user may hold a given working memory items in working memory, increasing the time a user may sustain attention on a particular stimuli, increasing the intensity of attention on a particular stimulus by user's ability to “block off” competing stimulus, increasing the intensity of attention on a particular stimulus by user's ability to selectively increase electrical activity in relevant DLPFC circuit(s) either by direct stimulation of DLPFC by sensing electrode set(s) 12C, or indirectly by targeted release of the neurotransmitter Dopamine (by Effector 15 for example), or by a modified version of sensing electrode set(s) 12C that has the capacity to inject dopamine directly focally into or in the vicinity of selected regions of the DLPFC, increasing the speed of parsing stimuli such as text, imagery etc., and storing it into long term memory.

In some system embodiments having the capacity to locally inject a neurotransmitter (such as, for example, dopamine) using an effector 15 configured as a localized cortical injector and a DLPFC sensing electrode set (such as, for example the sensing electrode set(s) 12C), the system may measure the amount of neurotransmitter (e.g. dopamine) released by effector 15, and present in the CNS location of interest and modulating it, based on present level of attention, working memory or other cognitive performance as measured by the BCI system performing sensing in the DLPFC.

In some system embodiments having the capacity to locally inject a neurotransmitter (such as, for example, dopamine) using an effector 15 configured as a localized cortical injector and a DLPFC sensing electrode set (such as, for example the sensing electrode set(s) 12C) and in which one of the auxiliary sensor (s) 18 includes sensor(s) for measuring a physiological parameter (such as, for example, the user's heart rate, the user's blood pressure, or any other suitable physiological or physico-chemical parameter of the user), while a certain amount of neurotransmitter is present at the region of interest in the DLPFC the system may modulate the amount of injected transmitter based on the cognitive performance of the user as well as by determining the physiological parameter's value and using it also for modulating or changing the amount of neurotransmitter delivered to the DLPFC by the effector 15.

Reference is now made to FIG. 3 which is a schematic block diagram illustrating an embodiment of a system for augmenting or enhancing or improving cognitive performance of a user, including one or more electrode sets for sensing neuronal activity in the dorsolateral prefrontal cortex (DLPFC) and for electrically stimulating several deep brain structures, in accordance with some embodiments of the systems of the present application.

The system 30 may include the processor/controller(s) 14, which may also be (optionally) suitably coupled or connected to the memory data storage device(s) 16 as disclosed in detail hereinabove. The processor/controller(s) 14 may also be (optionally) suitably coupled or connected to the (optional) auxiliary sensor(s) 18 and/or to the effector device(s) 15, as disclosed in detail hereinabove with respect to the systems 10 and 20 (of FIGS. 1 and 2, respectively).

The system 30 may also include one or more sensing and stimulating electrode set(s). The specific embodiment of the system 30 illustrated in FIG. 3 includes one or more sensing electrode set(s) 12C and one or more stimulating electrode set(s) 12D. The sensing electrode set(s) 12C and the stimulating electrode set(s) 12D are suitably connected to the processor/controller(s) 14.

The sensing electrode set(s) 12C is suitably coupled to the dorsolateral prefrontal cortex 39 and is disposed in the vicinity of the surface of the DLPFC 39 or within the DLPFC 39 (depending on the type of configuration used to implement the sensing electrode set(s) 12C). The first sensing electrode set(s) 12C may be used for sensing signals associated with neuronal activity in the DLPFC 39. For example, the sensing electrode set(s) 12C may be a flexible flat surface electrode array disposed on the surface of the DLPFC 39 for sensing/recording an electrocorticogram (Ecog) representing neuronal activities in the DLPFC 39 as is well known in the art and disclosed hereinabove However, the sensing electrode set(s) 12C may also be any other type of electrode set (s) as disclosed hereinabove for performing surface sensing, or for implantation within the DLPFC 39, or of the stent electrode array type as disclosed hereinabove. For example, the injectable flexible mesh type electrode arrays disclosed hereinabove may be used for sensing in the DLPFC. Furthermore, as such injectable flexible mesh type electrode arrays may be used for both stimulating and sensing, they may be used in system configurations in which such a mesh type electrode array may be used for both sensing and stimulating in the DLPFC. Such embodiments are described in more detail hereinafter.

The stimulating electrode set(s) 12D is suitably coupled to the striatum 41 and may be disposed within the striatum 41. The stimulating electrode set(s) 12D may be used for stimulating the striatum 41 or any part or parts of the striatum. For example, stimulation may be delivered by the stimulating electrode set(s) 12D to the caudate nucleus or to the putamen or to both the caudate nucleus and the putamen. The stimulation may be delivered at a single location or at multiple locations within the striatum or the parts thereof, or in neighboring regions that originate, or propagate one of the two central dopaminergic pathways. Other regions that may be stimulated include the substantia nigra, the nucleus accumbens and the dorsal striatum. Typically, regions that are part of the limbic structures within the mesocortical nigrostriatal, tuberoinfundibular and mesolimbic systems may also be stimulated to achieve the augmentation/enhancement/improvement in cognitive performance.

The stimulation of the striatum 41 is also referred to as stimulation of a deep brain structure as the striatum is a sub cortical region disposed relatively deep within the brain. It is noted that the term stimulation of a deep brain structure is also used to refer to the stimulation of any other brain structures and/or brain regions which are disposed below or internal to the cortex. For example, the sensing electrode set(s) 12C may be any type of penetrating multi electrode array capable of being implanted in a deep brain structure as is known in the art and as disclosed hereinabove with respect to electrode set types. It is noted that the stimulating electrode set(s) 12D may also be any type of electrode set(s) as disclosed hereinabove capable of performing stimulation (and/or sensing) of neuronal or neuronal population activity within deep brain structures. Such Electrode sets may include, for example, implantable injectable folded mesh electrode arrays for implantation within a deep brain structure, or of the stent electrode array type, which may be inserted through the vasculature into a blood vessel in the vicinity of or within the relevant deep brain structure, as disclosed hereinabove.

In operation of the system 30, the processor/controller(s) 14 may process the signals sensed by the sensing electrode set(s) 12C in the DLPFC 39 to detect pattern(s) of activity indicative of a cognitive task requiring learning, concentration and/or focusing of attention, and sustained attention, use of working memory or any of the other types of cognitive tasks disclosed in detail hereinabove.

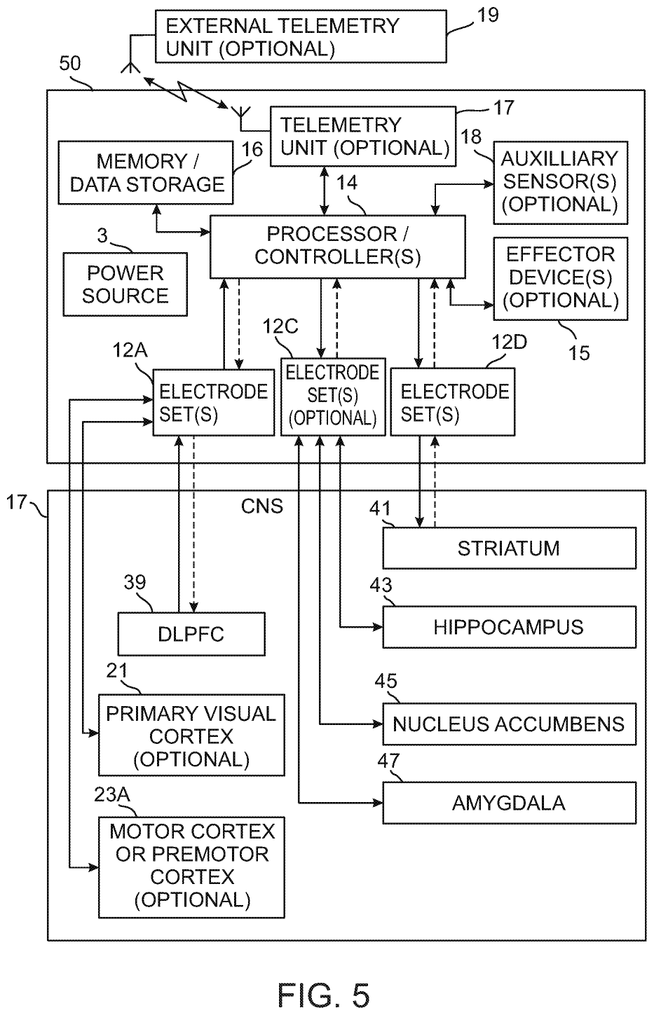

Once such a pattern (or patterns) is detected, the processor/controllers 14 (and any software program operating thereon) may control the timed application of a stimulating signal (or of a timed spatiotemporal stimulation pattern delivered to the striatum 41 (or to any other deep brain structures as illustrated in FIGS. 4 and 5 hereinafter). The stimulation of the striatum 41 may result in activation of the VTA region and it's dopaminergic systems projecting to many regions of the nervous system (including the PFC and DLPFC) which may result in improving and/or enhancing and/or augmenting and/or modulating the cognitive performance of sustaining attention, focusing attention, and even increasing motivation to perform the task which may in turn result in better or increased (augmented) cognitive performance which may include, inter alia, improved working memory performance, enhanced and more focused attention, increased learning and memory performance ceiling, faster user responses in performing cognitive tasks, and other types of cognition augmentation or enhancements. It is noted that the term “modulating” as used in the present application may also include diminishing certain cognitive functions, as it may also refer to selective “blocking” or attenuation of certain types of stimuli from distracting or drawing user's attention from concentrating on a certain task. Therefore the term modulating may also be interpreted as or may mean a selective “diminishing cognitive performance” as well as “ increasing or augmenting cognitive performance”.

The application of stimulation to the striatum responsive to detection of specific activity pattern(s) associated with preparation to perform cognitive tasks or with the presentation to the user of such cognitive tasks such as an associative memory task, a memorizing task, a comparison task or any other demanding cognitive task may preferably be automatic since the timing (and/or spatiotemporal characteristics) of such stimulation application is important to ensure affecting or enhancing the cognitive performance of the user. However, it may be possible to voluntarily use a certain level of stimulation at a certain frequency pattern and a certain intensity of stimuli, which may result in more neurotransmitter being released into mesolimbic (or other) dopaminergic pathways, in order to increase the general level of attention sustaining, when it is expected that enhanced cognitive performance may be needed for a certain period of time (see FIG. 5 for a specific example of a system capable of voluntarily controlling such stimulation, if desired).

It will be appreciated that while the system 30 is implemented as a wired system in which the various components of the system may be connected by wires to other components of the system 30, this is not obligatory, and some or all of the components of the system 30 or of any of the systems disclosed herein may be wirelessly connected to other components.

Reference is now mage to FIG. 4 which is a schematic block diagram illustrating a wireless embodiment of a system for augmenting or enhancing or improving cognitive performance of a user including one or more electrode sets for sensing neuronal activity in the dorsolateral prefrontal cortex (DLPFC) and for electrically stimulating one or several deep brain structures associated, inter alia, with learning, memory and regulation of attention, in accordance with some embodiments of the systems of the present application.

The system 40 may include the processor/controller(s) 14, which may also be (optionally) suitably coupled or connected to the memory data storage device(s) 16 as disclosed in detail hereinabove. The processor/controller (s) 14 is suitably connected or coupled to a wireless transceiver (TX1) 31 for wirelessly communication with other components of the system 40. The processor/controller(s) 14 may also be (optionally) suitably wirelessly coupled or connected to the (optional) auxiliary sensor(s) 18 through a suitable wireless transceiver 33 (TX3). The processor/controller(s) 14 may also be (optionally) suitably wirelessly coupled or connected to the (optional) effector device(s) 15 through a suitable wireless transceiver 34 (TX4). The system 40 may also include the stimulating electrode set(s) 12A as disclosed in detail hereinabove, which is capable of wirelessly communicating with the transceiver 31 through a transceiver 35 (TX5) connected to the stimulating electrode set(s) 12A, for sending signals to and/or receiving signal from the processor/controller(s) 14. The system 40 may also include the stimulating electrode set(s) 12D as disclosed in detail hereinabove, which is capable of wirelessly communicating with the transceiver 31 through a transceiver 32 (TX2) connected to the stimulating electrode set(s) 12D for sending signals to and/or receiving signal from the processor/controller(s) 14.

The stimulating electrode set(s) 12D is disposed and configured to deliver stimulation to one or more deep brain structures. Such deep brain structures may include, but are not limited to, the striatum, the caudate nucleus, the putamen, the nucleus accumbens, the locus ceruleus, the hippocampus, the amygdale, a deep brain structure of the meso-limbic system, a deep brain structure functionally participating in enhancing or facilitating learning, and/or memory and/or attention focusing, a sub-cortical region of the brain, and any combinations thereof.

In accordance with other embodiments of the systems of the present application, the stimulating electrode set(s) 12D may alternatively or additionally deliver stimulation to other (one or more) deep brain structures, such as hypothalamic structures or nuclei, thalamic structures or nuclei, and sub-thalamic structures or nuclei. Such stimulation may be delivered instead of or in addition to stimulation of the striatum and/or thalamic/hypothalamic/sub-thalamic structures.

The stimulating electrode set(s) 12A is suitably coupled to the dorsolateral prefrontal cortex 39 and is disposed in the vicinity of the DLPFC surface or within the DLPFC 39 (depending on the type of electrode set(s) used to implement the stimulating electrode set(s) 12A). The stimulating electrode set(s) 12A may be used for sensing signals associated with neuronal activity in the DLPFC 39. For example, the stimulating electrode set(s) 12A may be a flexible flat surface electrode array disposed on the surface of the DLPFC 39 for sensing/recording an electrocorticogram (Ecog) representing neuronal activities in the DLPFC 39 as is well known in the art and disclosed hereinabove However, the stimulating electrode set(s) 12A may also be any other type of electrode set(s) as disclosed hereinabove for performing surface sensing, or for implantation within the DLPFC 39, or of the stent electrode array type as disclosed hereinabove.

The stimulating electrode set(s) 12D is suitably coupled to the one or more deep brain structure(s) 37 as disclosed hereinabove and may be disposed within or near one or more of the deep brain structure or structures, depending on the type of stimulating electrode set(s) 12D being used. The stimulating electrode set(s) 12D may be used for stimulating the deep brain structure(s). The stimulation of the deep brain structure(s) 37 is also referred to as stimulation of deep brain structures as the deep brain structure(s) 37 are sub cortical regions disposed relatively deep within the brain. It is noted that the term stimulation of deep brain structures is also used to refer to the stimulation of any other brain structures and/or brain regions which are disposed below or internal to the cortex and/or deep within the brain. For example, the stimulating electrode set(s) 12D may be any type of penetrating multi electrode array capable of being implanted in a deep brain structure as disclosed hereinabove. It is noted that the stimulating electrode set(s) 12D may also be any type of electrode set(s) as disclosed hereinabove capable of performing stimulation (and/or sensing) of neuronal or neuronal population activity within deep brain structures. Such electrode set(s) may include, for example, implantable injectable folded mesh electrode arrays for implantation within a deep brain structure, or of the stent electrode (also referred to as a “stentrode”, hereinafter) or stent electrode array type, which may be inserted through the vasculature into a blood vessel in the vicinity of or within the relevant deep brain structure, as disclosed hereinabove.

In operation of the system 40 may operate similarly to the operation of the system 30, except that the signals sensed by the stimulating electrode set(s) 12A are wirelessly communicated to the processor/controller 14, the stimulation signals or stimulation commands to the stimulating electrode set(s) 12D are wirelessly communicated from the processor/controller(s) 14 to the stimulating electrode set(s) 12D and the communication between the auxiliary sensor(s) 18, the effector Devices(s) 15 and the processor/controller(s) 14 may be wirelessly performed. It is noted that the stimulating electrode set(s) 12D may (if necessary, due to the wireless communication capability) include all the necessary circuitry to receive and interpret stimulation commands from the processor/controller(s) 14, and may also include a built in power source for powering the delivering the stimulation.

It will be appreciated that the stimulation may not only be delivered to the striatum (or to one or more parts of the striatum) as disclosed with respect to the system 30 of FIG. 3 hereinabove, but may also be delivered to any number of deep brain regions which may be useful to enhance or improve the above disclosed cognitive enhancement or cognitive improvements.

Reference is now made to FIG. 5 which is a schematic block diagram illustrating a system for augmenting or enhancing or improving cognitive performance of a user including several electrode set(s) for sensing neuronal activity in the dorsolateral prefrontal cortex (DLPFC) cortical region and (optionally) in other cortical regions and for electrically stimulating one or several deep brain structures associated, inter alia, with learning, memory and regulation of attention, in accordance with some embodiments of the augmented/enhanced cognition systems of the present application.

The system 50 may include the processor/controller(s) 14, the memory/data storage device(s) 16, the auxiliary sensor(s) 18, the effector device(s) 15, the stimulating electrode set(s) 12A, the stimulating electrode set(s) 12D, connected as disclosed hereinabove in FIGS. 1-3.

The system 50 may also include a sensing electrode set(s) 12C, suitably connected to the processor /controller(s) 14. The sensing electrode set(s) 12C may be used for stimulating of the striatum 41 as disclosed in detail hereinabove with respect to FIG. 4. The stimulating electrode set(s) 12D may be used to deliver stimuli to the hippocampus 43, the nucleus acumbens 45 and the amygdala 47. The stimulation of each of the deep brain structures which are stimulatable by the stimulating electrode set(s) 12D, may be performed in accordance with one or more selected spatiotemporal patterns which are empirically found to enhance the performance of any of the cognitive tasks disclosed in detail hereinabove.

The stimulating electrode set(s) 12A may sense neuronal activity related signals in both of the DLPFC 39 and in the motor (and/or premotor) cortex 23A, similar to the operation of the stimulating electrode set(s) 12A as disclosed with respect to FIG. 2. The stimulating electrode set(s) 12A may also be used to stimulate the primary visual cortex 21 as disclosed in detail with respect to the system 20 of FIG. 2.

In some embodiments, the system and may include a (optional) telemetry unit 17 for wirelessly communicating with an external telemetry unit 19. The telemetry unit 17 may bidirectionally communicate with the processor/controller(s) 14 and may be used to wirelessly communicate data from the memory/data storage 16 and/or from the processor/controller(s) 14 to the external telemetry unit 19 for further processing, further storage and for displaying the data. The External telemetry unit 19 may also be used to wirelessly send signals to the processor/controller(s) 14 for controlling the operations thereof and/or for reprogramming the software operating the controller processor(s) 14. For example, when some or all of the processor/controllers(s) 14, the electrode sets 12A, 12B and 12C, the memory/storage 16, the auxiliary sensor(s) 18 and the effector device(s) 15 are implanted intracranially, the telemetry unit 17 may be intracranially disposed for wireless communication with an external telemetry unit 19 as disclosed hereinabove.

The system 50 may be operated to augment and/or improve the performance of cognitive tasks as disclosed in detail hereinabove as well as operate to control the performance of general or specific computing tasks as disclosed hereinabove. For example, by interacting with a virtual GUI perceived by the user as a result of stimulation of the primary visual cortex 21 (and/or other parts of the visual cortex), the user may voluntarily activate or deactivate, as per his need, the operation of software program(s) controlling the sensing in the DLPFC 39, and/or program(s) controlling the stimulation of the Striatum 41, and/or the hippocampus 43, the nucleus acumbens 45 and the amygdala 47 by the sensing electrode set(s) 12C and/or the stimulating electrode set(s) 12D. The user may also control the intensity of stimulation of each of the deep brain structures being stimulated for changing and/or modulating the enhancing effect of the stimulation on the cognitive performance as the need arises.

Methods and devices for stimulating the striatum as well as for stimulating other deep brain structures using several types of stimulating electrode sets or other stimulating devices are well known in the art. For example, the following publications disclose, inter alia, such methods and devices for performing stimulation of deep brain structures are disclosed in detail in the following publications incorporated herein by reference in their entirety:

1. Husam A. Katnani, Shaun R. Patel, Churl-Su Kwon, Samer Abdel-Aziz, John T. Gale & Emad N. Eskander. “Temporally Coordinated Deep Brain Stimulation in the Dorsal and Ventral Striatum Synergistically Enhances Associative Learning.”, Scientific Reports 6, Nature, Article number: 18806 (2016).

2. J. T. Gale, K. H. Lee, R. Amirnovin, D. W. Roberts, Z. M. Williams, C. D. Blaha & E. N. Eskandar. “Electrical Stimulation-Evoked Dopamine Release in the Primate Striatum. Stereotactic and Functional Neurosurgery.”, Karger Medical and Scientific Publishers, Vol. 91, No. 6, 2013.

3. Sarah K. B. Bick & Emad N. Eskandar. “Neuromodulation for restoring memory.”, Neurosurgical Focus, JNS Journal of Neurosurgery, May 2016, Vol. 40, No. 5, Page E5.

4. Nikolaos Makris, Yogesh Rathi, Palig Mouradian, Giorgio Bonmassar, George Papadimitriou, Wingkwai I. Ing, Edward H. Yeterian, Marek Kubicki, Emad N. Eskandar, Lawrence L. Wald, Qiuyun Fan, Aapo Nummenmaa, Alik S Widge & Darin D. Dougherty. “Variability and anatomical specificity of the orbitofrontothalamic fibers of passage in the ventral capsule/ventral striatum (VC/VS): precision care for patient-specific tractography-guided targeting of deep brain stimulation (DBS) in obsessive compulsive disorder (OCD). “, Brain Imaging and Behavior, December 2016, Volume 10, Issue 4, Pp. 1054-1067.

5. Darin D. Dougherty, Ali R. Rezai, Linda L. Carpenter, Robert H. Howland, Mahendra T. Bhati, John P. O'Reardon, Emad N. Eskandar, Gordon H. Baltuch, Andre D. Machado, Douglas Kondziolka, Cristina Cusin, Karleyton C. Evans, Lawrence H. Price, Karen Jacobs, Mayur Pandya, Timothey Denko, Audrey R. Tyrka, Tim Brelje, Thilo Deckersbach, Cynthia Kubu & Donald A. Malone Jr., “A Randomized Sham-Controlled Trial of Deep Brain Stimulation of the Ventral Capsule/Ventral Striatum for Chronic Treatment-Resistant Depression”. Biological Psychiatry, Aug. 15, 2015, Vol. 78, Issue 4, Pp. 240-248.

6. John T. Gale, Donald C. Shields, Yumiko Ishizawa & Emad N. Eskandar. “Reward and reinforcement activity in the nucleus accumbens during learning.”, Frontiers in Behavioral Neuroscience, 3 Apr. 2014,1 www(dot)dx(dot)doi(dot)org/10(dot)3389/fnbeh(dot)2014(dot)00114.

7. Jesse J. Wheeler, Keith Baldwin, Alex Kindle, Daniel Guyon, Brian Nugent, Carlos Segura, John Rodriguez, Andrew Czarnecki, Hailey J. Dispirito, John Lachapelle, Philip D. Parks, James Moran, Alik S. Widge, Darin D. Dougherty & Emad N. Eskandar. “An implantable 64-channel neural interface with reconfigurable recording and stimulation.”, IEEE Xplore Digital Library, www(dot)ieeexplore(dot)ieee(dot)org/document/7320208.

8. Lei Hamilton, Marc McConley, Kai Angemueller, David Goldberg, Massimiliano Corba, Louis Kim, James Moran, Philip D. Parks, Sang Chin, Alik S Widge, Darin D. Dougherty & Emad N. Eskandar. “Neural signal processing and closed-loop control algorithm design for an implanted neural recording and stimulation system.”, IEEE Xplore Digital Library, www(dot)ieeexplore(dot)ieee(dot)org/document/7320207.

9. Beata Jarosiewicz, Anish A. Sarma, Daniel Bacher, Nicolas Y. Masse, John D. Simeral, Brittany Sorice, Erin M. Oakley, Christine Blabe, Chethan Pandarinath, Vikash Gilja, Sydney S. Cash, Emad N. Eskandar, Gerhard Friehs, Jaimie M. Henderson, Krishna V. Shenoy, John P. Donoghue & Leigh R. Hochberg. “Virtual typing by people with tetraplegia using a self-calibrating intracortical brain-computer interface.” Science Translational Medicine, American Association for the Advancement of Science. Vol. 7, Issue 313, 11 Nov. 2015.

Methods and devices for sensing electrical cortical activity in various cortical regions and for are also well known in the art, such as, but not limited to the references cited hereinabove. Any of these methods and electrode set(s) devices known in the art and described in the references cited herein may be used for sensing/recording neuronal activities in the DLPFC. For example, the injectable flexible mesh electrodes such as the one disclosed by Tian Ming Fu et al. (Nature methods, 2016) may be used by implantation of such mesh electronics within the DLPFC. Another method may use the less invasive flat flexible surface electrode arrays. Other systems and methods may make use of stent electrode arrays (stentrodes) as disclosed hereinabove.

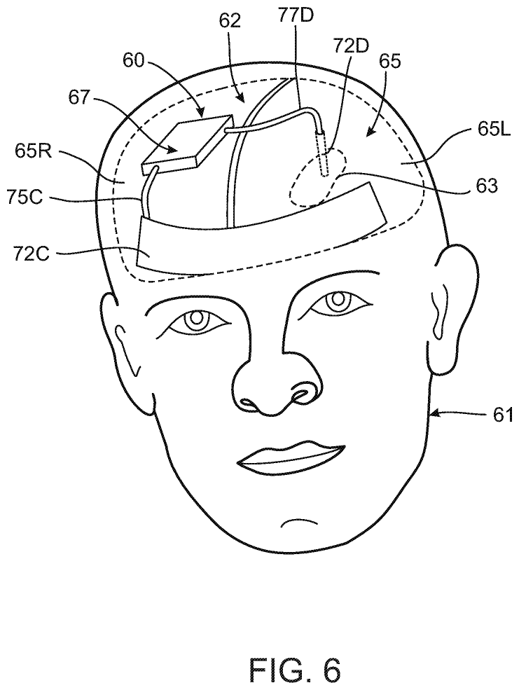

Reference is now made to FIG. 6, which is a schematic diagram illustrating an intracranial system for augmenting or enhancing or improving cognitive performance of a user, disposed within the cranium of the user, in accordance with some embodiments of the systems of the present application. The system 60 is shown disposed intra-cranially within the head 61 of a user. Part of the cranium is made “transparent in the schematic drawing to show the brain 62 of the user schematically illustrating the cortex 65 which includes the left cortical hemisphere 65L and the right cortical hemisphere 65R. The striatum (corpus striatum) 63 is schematically illustrated in a dashed line to indicate that the striatum is a subcortical brain region (a deep brain structure) which lies under the cortex 65. The system 60 includes an electronic circuitry module 67, a sensing electrode set 72C suitably electrically connected to the electronic circuitry module 67 by a communication line 75C and a stimulating electrode set 72D suitably electrically connected to the electronic circuitry module 67 by a communication line 77C.

The components of the system 60 may be inserted into the intracranial space above the brain 62 through an access opening (the opening not shown for the sake of clarity of illustration) made in the cranial bone by performing craniotomy (including but not limited to, manual craniotomy methods, stereotactic craniotomy methods, automatic robotic stereotactic craniotomy, or any other type of suitable craniotomy method known in the art). After insertion of the components of the system 60 into the intracranial space, the opening in the skull may be sealed as is known in the art.

The sensing electrode set 72C may be a thin flexible surface electrode array adapted for sensing and/or recording an Ecog (and may be disposed epidurally or under the dura in contact with the cortical surface). The sensing electrode set 72C may be used for sensing and/or recording an Ecog from the DLPFC of the left cortical hemisphere 65L or from the DLPFC of the right cortical hemisphere 65R or from the DLPFC of both the right cortical hemisphere 65R and the left cortical hemisphere 65L (depending, inter alia, on the total area and positioning of the sensing electrode set 72C).

It is noted that the sensing of Ecog signals using Ecog electrode arrays, may be performed using standard Ecog sensing methods. For example one of the electrodes in the array may be used as a reference electrode. Alternatively, an electrode facing the skull may be used as a reference electrode. Alternatively, a special electrode in the implant may serve as the reference electrode.

In accordance with some embodiments of the system 60, the sensing electrode set 72C is used to sense and/or record an Ecog from one (either the left or the right) DLPFC or from a part or a portion of the DLPFC. In accordance with some embodiments of the system 60, the sensing electrode set 72C may be used to sense and/or record an Ecog from both the left DLPFC and the right DLPFC (or from a part or portion of each of the left DLPFC and right DLPFC).

In accordance with some embodiments of the system 60, the sensing electrode set 72C may be large enough to sense and/or record an Ecog from the right and/or the left DLPFC as well as from one or from several cortical regions other than the DLPFC (including but not limited to, the primary visual cortex, other areas of the visual cortex, the somatosensory cortex, the auditory cortex, the motor cortex, Brodmann area (BA) 17 (approximately corresponding to primary visual cortex—V1), BA 18 (approximately corresponding to secondary visual cortex—V2), BA 19 (approximately corresponding to associative visual cortex—V3, V4 and V5), BA 7 (visuo-motor coordination area), BA 6 (premotor cortex and supplementary motor cortex area), BA 5 (somatosensory association cortex) and BA 4 (primary motor cortex).

Preferably, the sensing electrode set 72C may be a Medium to high resolution multi electrode array having several hundred to several thousand sensing electrodes, respectively, but electrodes set(s)/array(s) with a smaller number of electrodes (in the range of 50-150 electrodes per BCI) may also be used. The communication line 75C may have multiple electrically isolated electrically conducting wires therein (not shown) connecting each electrode of the sensing electrode set 72C to the electronic circuitry module 67. However, electrode multiplexing methods may also be used in some embodiments of the system, as is known in the art, to allow multiple electrodes to be periodically sampled through the same electrically conducting wire, in order to reduce the number of required wires within the communication line 75C.

The stimulating electrode set 72D may be any type of stimulating electrode set(s) capable of being implanted within a deep brain structure. For example. The stimulating electrode set 72D of the system 60 of FIG. 6 may be an elongated thin flexible bundle of electrically conducting electrodes (such as, for example, a bundle of several electrically isolated tungsten electrodes having exposed electrically conducting tips arranged in a staggered arrangement at the tip of the electrode bundle). Each of the electrodes (not shown) in the bundle may be suitably electrically connected to the electronic circuitry module 67 by a single isolated electrically conducting wire passing through the communication line 77D. The tip of the stimulating electrode set 72D may be surgically implanted within a region (or regions) of the striatum 63 (such as, but not limited to, the caudate nucleus, the putamen, the dorsal striatum, the ventral striatum or any combinations thereof), as is well known in the art. Methods that may be used for implantation of the stimulating electrode set 72D may include, but are not limited to, manual or semi-manual stereotactic electrode implantation methods, automatic robotic stereotactic electrode implantation methods, or any other type of suitable electrode methods known in the art).

However, it is noted that the stimulating electrode set 72D (as well as the sensing electrode set 72C) may be also implemented as other different types of electrode set(s). For example, in accordance with some embodiments of the systems of the present application, the electrode sets 72C and/or 72D may be a flexible injectable mesh electrode array as disclosed in detail by Lieber et al. in the following references:

1. Chong Xie, Jia Liu, Tian-Ming Fu, Xiaochuan Dai, Wei Zhou & Charles M. Lieber. “Three-dimensional macroporous nanoelectronic networks as minimally invasive brain probes.”, Nature Materials, Vol. 14, December 2015, Pp. 1286-1292.

2. Guosong Hong, Tian-Ming Fu, Tao Zhou, Thomas G. Schuhmann, Jinlin Huang, & Charles M. Lieber. “Syringe Injectable Electronics: Precise Targeted Delivery with Quantitative Input/Output Connectivity”, Nano Letters, Vol. 15, August 2015, Pp. 6979-6984. DOI: 10.1021/acs.nanolett.5b02987. and

3. Jia Liu, Tian-Ming Fu, Zengguang Cheng, Guosong Hong, Tao Zhou, Lihua Jin, Madhavi Duvvuri, Zhe Jiang, Peter Kruskal, Chong Xie, Zhigang Suo, Ying Fang & Charles M. Lieber. “Syringe-injectable electronics.”, Nature Nanotechnology, Vol. 10, July 2015, Pp. 629-636. DOI: 10.1038/NNANO.2015.115.

In accordance with some embodiments, the stimulating electrode set 72D may be a stentrode or a stentrode array implanted within a blood vessel that is part of the vasculature within the striatum 63, as disclosed in detail by Oxley et al. hereinabove (Nature biotechnology, Vol. 34, No. 3, February 2016). In such a case, the communication line 77D may be replaced by suitable (preferably, ultrasonic) wireless transceivers, suitably connected to the stimulating electrode set 72D and to the electronic circuitry module 67 (the transceivers are not shown in FIG. 6, but see FIG. 4 hereinabove for detail).

The electronic circuitry module 67 may include the processor/controller(s) 14 (of FIG. 3) and the memory/data storage 16 (of FIG. 3) that may be connected to the processor/controller(s) 14 as disclosed in detail in of FIG. 3 hereinabove. As the electronic circuitry module 67 is intracranially implanted, it may also include a power source (not shown in detail in FIG. 6, for the sake of clarity of illustration). The power source included in the electronic circuitry module 67 may be any suitable miniature power source known in the art. However, preferably (but not obligatorily), the power source may be a wireless power harvesting device which may receive and store power transmitted to it from a power transmitting device (not shown) disposed outside or on the body of the user.

Such wireless power transmitting and receiving systems are well known in the art, are not the subject matter of the present application and are therefore not disclosed in detail hereinafter. Briefly, such systems may include a piezoelectric material based receiver coupled to a suitable current rectifying circuitry and an electrical storage device (a capacitor, a super-capacitor, a rechargeable electrochemical cell and the like). Such a power receiver may harvest ultrasonic energy transmitted to it from an ultrasound transmitter outside the body, turn the ultrasonic energy into an electrical current and store the electrical energy in the above described electrical energy storage devices. In other examples, may include electromagnetic radiation based systems including a harvesting electrically conducting coil coupled to current rectifying circuitry that feeds an electrical energy storage device as disclosed hereinabove. The external transmitter is a generator of electromagnetic radiation which transmits the electromagnetic radiation (typically, through another coil, external to the body of the user) required for energizing the receiver coil by induction.

In operation, the sensing electrode set 72C senses electrical activity associated with electrical activity of neurons within the DLPFC (either left DFPLC or the right DFPLC or the left and the right DFPLC). If the sensing electrode set 72C is an Ecog electrode array, the sensed electrical activity may be an Ecog. If the sensing electrode set 72C, is a Utah array type electrode array or a mesh type electrode array, the sensed electrical activity may include extracellularly sensed field potentials from individual neuron(s) or field potentials resulting from summed (superimposed) extracellularly recorded action potentials from multiple neurons as well as extracellularly sensed electrical activity (from neuronal axons, dendrites and soma). The sensed signals are fed to the electronic circuitry module 67.

The electronic circuitry module 67 may process the electrical signals sensed by the sensing electrode set 72C to detect specific spatiotemporal patterns of electrical activity associated with the performance of cognitive tasks (such as, for example, tasks requiring attention focusing and/or sustained attention, and/or learning, and/or activation of working memory (WM), and/or any other complex cognitive task as disclosed in detail in the present application). Such specific spatiotemporal electrical activity patterns may precede the actual performance of such a cognitive task and/or may be associated with the user's intention to perform the cognitive task or with the presentation of such a cognitive task to the user.

If such a specific pattern is detected by the system 60, the electronic circuitry module 67 delivers stimulation to the striatum 63, or to one or several parts of the striatum 63 by applying suitable stimulating electrical current pulses to the Striatum 63 through electrodes of the stimulating electrode set 72D. The stimulation delivered to the striatum 63 may be precisely timed with respect to the time of detection of the specific spatiotemporal pattern(s) of electrical activity associated with the performance of cognitive tasks.

The stimulation of the striatum 63 in response to the detected patterns results in timed activation of the VTA and deeper brain structures that release dopamine which may reinforce connections between relevant neurons that are strengthened during that process of learning the new cognitive task. This timed stimulation may result in augmentation (improvement) of attention focusing and attention sustaining, augments (or improves) the rate of learning and memory performance.

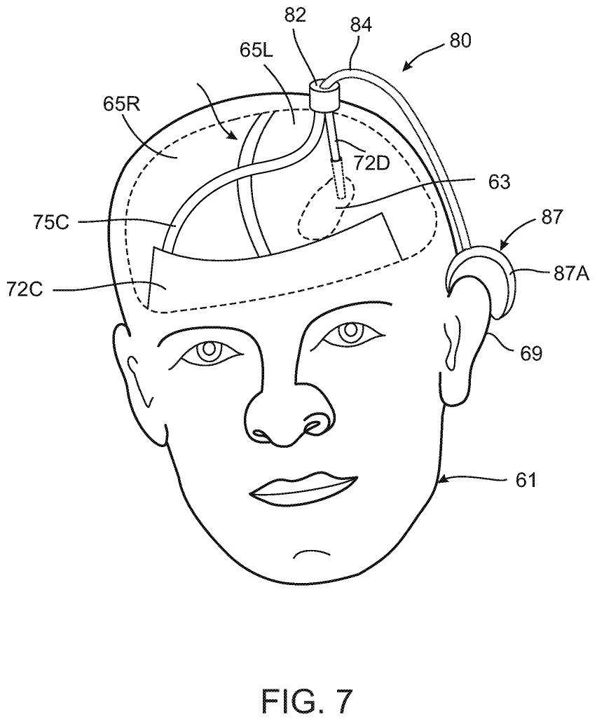

Reference is now made to FIG. 7, which is a schematic diagram illustrating a system for augmenting or enhancing or improving cognitive performance of a user, having some system components disposed within the cranium of a user and some other components of the system disposed outside the cranium of the user, in accordance with some embodiments of the systems of the present application.

The system 80 may include the sensing electrode set 72C and the stimulating electrode set 72D which may be constructed and operated as disclosed in detail hereinabove with respect to FIG. 6. However, in contrast to the system 60, the communication line 75C, and the communication line 77C of the system 80 pass through a suitable cranial connector 82 and exit through an opening in the connector 82 to pass outside the skull within a an extra-cranial communication cable 84. The extra-cranial communication cable 84 extends to an extra-cranial electronic module 87. The extra-cranial electronic module 87 may include a suitable processor/controller, a memory and data storage unit, an (optional) wireless transmitter(s) and/or wireless receiver(s) and/or wireless transceiver(s) and a suitable power source for energizing all the included circuitry that may be disposed within a housing 87A of the electronic module 87.

The processor/controller, the memory/data storage, any transmitters, receivers and/or transceivers and the power source which may be included in the housing 87A are not shown in detail in FIG. 7 for the sake of clarity of illustration, but may be similar to the processor/controller 67, the memory/data storage 16 and the transmitters/transceivers TX1-TX5 and the power source 3 disclosed in detail hereinabove).

The electronic module 87 may be convenient in cases in which some of the components therein may be too large or cumbersome for intracranial implantation. The electronic module 87 may also enable a relatively large power source to be included within the electronics module (such as a replaceable primary electrochemical cell, a rechargeable (secondary) electrochemical cell, or any other suitable power source.

The advantage of the system 80 is that they are easier and less costly to implement due to less stringent requirements for component microminiaturization and the availability of more space to accommodate extra-cranial components.

It is noted that while the housing 87A of the electronic module 87 may be shaped similar to a miniature hearing aid and worn behind the ear 69 to minimize visibility, this is not obligatory and other types and shapes of the housing 87A may be used that may be carried by the user by attachment to other body parts or attached to a garment worn by the user. For example, the housing of the electronic module may be shaped like spectacles to be worn by the user, or attached to a suitable headband worn by the user.

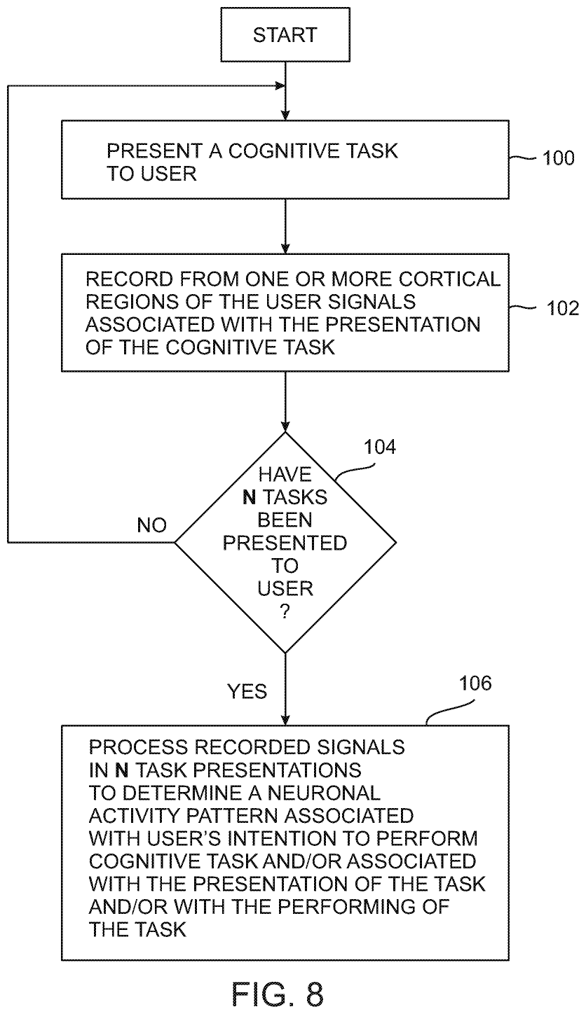

Reference is now made to FIG. 8, which is a schematic flow chart illustrating steps of a method for training and/or calibrating a system for augmenting or enhancing or improving cognitive performance of a user, in accordance with some embodiments of the methods of the present application.

Typically, the training method may be performed on a normal person or on a patient after implantation/installation of any of the intelligence enhancing systems disclosed in the present application. The software program operating on the processor/controller 14 may include a training module or subroutine that may be activated/started by using any suitable user interface (such as, for example, by any type of GUI of any type of computer external to the system and in communication with the processor/controller 14, or by a “virtual” graphic user interface presented to the user by direct stimulation of one or more regions of the visual cortex as disclosed hereinabove with respect to FIG. 2.

In operation of the training method, the system presents a cognitive task to the user (step 100). The cognitive task may be any suitable cognitive task that requires user attention focusing, such as, for example, a learning task, a memorizing task, a task associated with visual or audio discrimination, or any other suitable type of cognitive task as disclosed in detail hereinabove. Before, during, and after the presentation of the task, the system may record from one or more cortical regions of the user (such as for example, from one or more of prefrontal cortex (PFC), a part of the PFC, a dorsolateral prefrontal cortex (DLPFC), a part of the DLPFC, a temporoparietal cortex (TPC), a part of the TPC, an inferior frontal gyms (IFG), a part of the IFG, the temporal parietal junction (TPJ), a part of the TPJ, parietal lobule and any combinations of these regions) signals associated with neuronal activity associated with the presentation of the cognitive task presented to the user (step 102). In step 102, the system may also record signals associated with neuronal activity before the presentation of the task in order to study the characteristics of background neuronal activity in the absence of a cognitive task.

The system may then check if the number of cognitive tasks presented to the user in the training session is N (step 104), wherein N may be a user set, or physician set, or preprogrammed positive integer. If the number of cognitive tasks presented to the user is not N, the system returns control to step 100 for presenting the next cognitive task to the user. If the number of cognitive tasks presented to the user is N, the system terminates the presentation of cognitive tasks and processes the signals recorded for all N task presentations (step 106). In step 106, the system may determine or compute a template representing a neuronal activity pattern associated with the user's intention to perform a cognitive task and/or a template representing a neuronal activity pattern associated with the presentation of a cognitive task and/or associated with the actual performing of the cognitive task.

The use of N repetitions of similar (but not necessarily identical) cognitive task allows to extract a typical template or indication (decision criterion) based on multiple recorded signals that may be used by the system as an indication for identifying when a cognitive task requiring focusing of attention or enhancing the attention span of the user is presented to the user.

It is noted that many computational methods and algorithms are known for extracting a typical template from signals associated with neuronal activity recorded before during and after cognitive events presented to an experimental animal or a human patient or tested human subject. Such methods may include, for example, kernel analysis, principal component analysis, spectral analysis methods (particularly useful for analysis of Ecog type signals), common spatial patterns method (CSP), Analytic CSP (ACSP), time domain analytic methods, Frequency Domain analytic methods supervised pattern classification, cluster seeking methods, likelihood functions and statistical decision, and any other suitable pattern detection methods/algorithms. For example, such template pattern detection may be performed as described in any of the references cited herein.

The typical or representative pattern or template or indication or decision criterion determined in step 106 may be then stored in the memory/data storage 16 of the system for later use by the system in the identification of events requiring delivery of stimulation to deep brain structures by the system to achieve intelligence augmentation or intelligence enhancement.

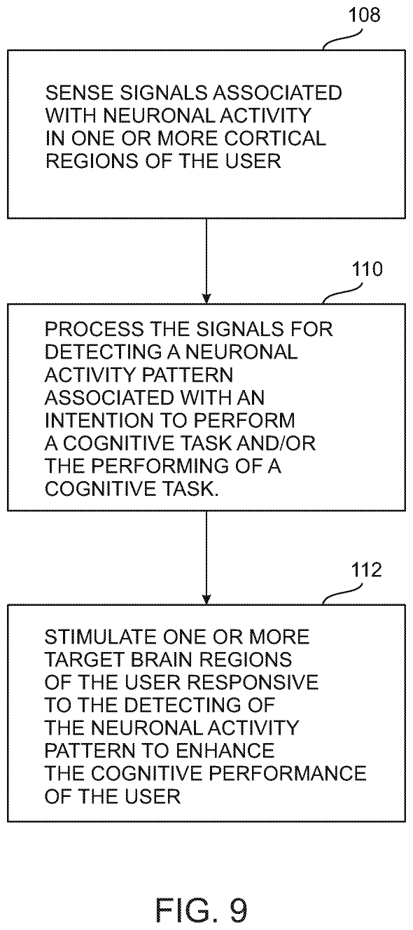

Reference is now made to FIG. 9, which is a schematic flow chart illustrating steps of a method for augmenting or enhancing or improving cognitive performance of a user, in accordance with some embodiments of the methods of the present application. The method may be used for enhancing and/or augmenting intelligence and for augmenting or enhancing cognitive performance of a normal user and/or for improving the cognitive performance of patients suffering from cognitive impairment due to psychological and/or neuropsychological and/or neurological disorders.

The method may be performed using any of the systems disclosed hereinabove. The system senses signals associated with neuronal activity in one or more cortical regions (such as, for example, one or more of the prefrontal cortex (PFC), a part of the PFC, the dorsolateral prefrontal cortex (DLPFC), a part of the DLPFC, the temporoparietal cortex (TPC), a part of the TPC, the inferior frontal gyrus (IFG), a part of the IFG, the temporal parietal junction (TPJ), a part of the TPJ, and any combinations of these cortical regions) of the user or patient (step 108). The sensing may be performed in real time or in quasi-real time. For example, by continuously digitizing the signals received from the electrode set(s) recording from the PFC (such as, for example the sensing electrode set(s) 12C or the stimulating electrode set(s) 12A, of FIGS. 2 and 3, respectively).

The system processes the sensed signals for detecting a neuronal activity pattern that is an indication of an intention of the user to perform a cognitive task and/or associated with the performance of a cognitive task by the user (step 110). Many types of methods and/or algorithms for detecting a neuronal activity pattern may be used in the step of processing the sensed signals. Exemplary detection methods may include but are not limited to, kernel analysis, principal component analysis, spectral power analysis methods (particularly useful for analysis of Ecog type signals), phase lag analysis, common spatial patterns method (CSP), Analytic CSP (ACSP), time domain analytic methods, Frequency Domain analytic methods, supervised pattern classification, cluster seeking methods, likelihood functions and statistical decision, and any other suitable pattern detection methods/algorithms.

For the detection of step 110, the pattern detection methods may use a user specific pattern (template) or indication or decision criterion obtained in one or more training session performed by the user as disclosed hereinabove in detail for the method of FIG. 8. The template (s) or indication or decision criterion resulting from such system training sessions may be stored in the memory/data storage of the system (such as, for example the memory/data storage unit 16). The detection may be performed by any suitable method for pattern recognition, or by performing a comparison of a measured or computed parameter value with an empirically determined parameter threshold value. Detection methods may include, but are not limited to, digital or analog template matching methods, or computations comparing the value of a decision criterion (such as for example a threshold value) with a current value of a computed parameter determined from depending, inter alia, on the type of circuitry included in the processor/controller 14 (digital, analog or hybrid/digital/analog circuitry), the speed of computation (computational power) available to the processor/controller 14, and other considerations.

In response to the detection of a neuronal activity pattern associated with an intention to perform a cognitive task and/or with the actual performing of a cognitive task, the system stimulates one or more target brain regions of the user to augment or enhance or improve the cognitive performance of the user (step 112). The target brain regions may include one or more deep brain structures or one or more cortical regions or a combination of one or more deep brain structures and one or more cortical regions.

The deep brain structure to which stimulation is delivered in step 112, may be the striatum (corpus striatum) of the user and the stimulation may be delivered by the electrodes implanted in (if using implanted stimulating electrode(s), or near (if using a stentrode for stimulation) the VTA or the striatum (and/or any other deep brain structures that are stimulated by the system). Examples of such stimulating electrodes may include but are not limited to, the sensing electrode set(s) 12C of FIG. 3. The stimulating electrode set(s) 12D of FIG. 4. The stimulating electrode set(s) 12A of FIG. 5, the electrode set 72D of FIGS. 6-7, or any other suitable electrode set (s)capable of stimulating deep brain structures and/or cortical regions.

The stimulation of the deep brain structure may preferably be electrical stimulation performed by delivering suitable electrical current pulses to the deep brain structure(s) being stimulated. However, other stimulation methods may also be used such as, for example, photonic stimulation (using optogenetic methods), transcranial frequency interference stimulation (TFI) methods (as disclosed by the article of Nir Grossman et al referenced hereinabove) or by an intracranial frequency interference stimulation (IFI) or any other type of suitable neuronal tissue stimulation method that may be applied to a deep brain structure. The stimulation of cortical regions (if stimulated) may be performed by using the sensing electrodes (such as the sensing electrodes of an Ecog array or of a Utah array or of any other type of electrode set(s) used for sensing in the cortical regions).

In the method, the steps of sensing (step 108) and processing (step 110) may be performed continuously, such embodiments of the method with continuous sensing may be used in systems in which the stimulation artifacts may be sufficiently attenuated by suitable signal conditioning methods such as, for example high pass filtering, low pass filtering or band pass filtering or by suitable computational methods performed during the processing of the digitized sensed signals (such computational methods may make use of empirically determined stimulation artifacts parameters which may be determined for each individual user by testing or system training sessions using actual stimulation delivered to the target brain regions in a resting state of the user.

In accordance with some embodiments of the method, the step of stimulating (step 112) may be performed automatically in response to detecting a neuronal activity pattern or indication associated with the intention to perform a cognitive task and/or the performing of such a cognitive task. In some embodiments of the methods disclosed herein, the sensing is nor performed continuously but is stopped during the stimulation of the target brain regions to avoid stimulation artifacts from interfering with the sensed signals, in such embodiments, the sensing is continued after the stimulation of the target brain regions is terminated.

In accordance with other embodiments of the method, the step of stimulating (step 112) may be under the control of the user such that the user may voluntarily disable or enable the step of stimulating (step 112). For example, if a user of the system, encounters a situation where he or she may need to perform cognitive tasks, the user may enable the step of stimulating (step 112), in order to enhance his/her cognitive performance. If the user is in a period which does not require enhanced cognitive performance (such as, for example, resting, sleeping, exercising, or other activities), the user may disable the step of stimulating. Such disabling or enabling of the step of stimulating may be performed by using any user interface and may also be performed by the user voluntary using a perceived virtual image of a user interface as disclosed in detail hereinabove with respect to systems of FIG. 2 and FIG. 5.

Similarly, in patients in which the system is installed in order to treat a cognitive impairment or dysfunction (such as, for example, patients with ADD), the physician or other caregiver may be able to disable (and enable) the step of stimulation of the method when necessary (such as, for example, when performing a training session with the system and the user as disclosed in detail hereinabove with respect FIG. 8). This type of enabling/disabling of the stimulation may be performed, for example, by using a suitable GUI displayed on an external computer that is in communication with the controller /processor 14 of the system.

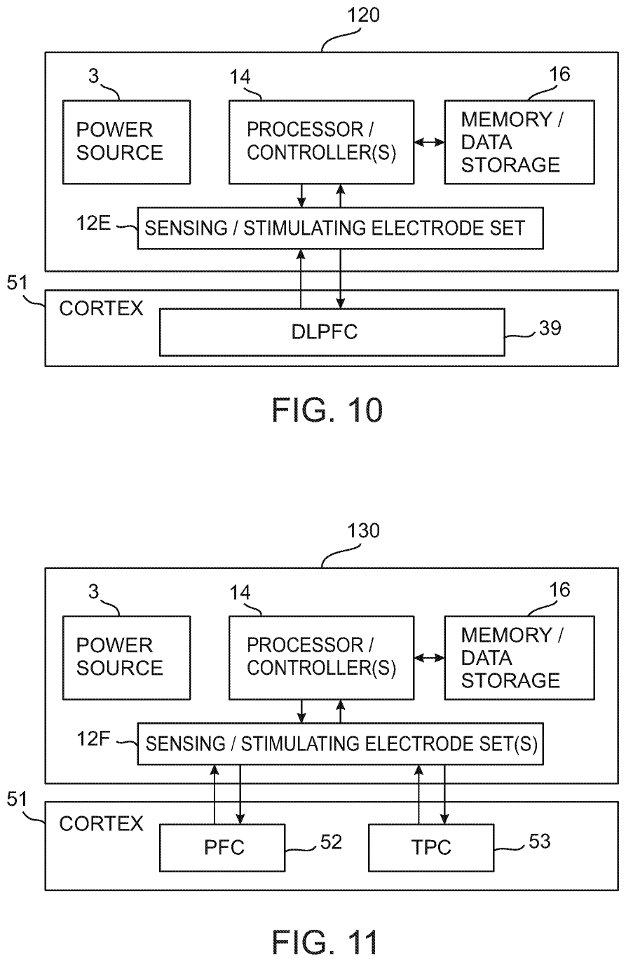

Reference is now made to FIG. 10 which is a schematic block diagram illustrating a system for augmenting or enhancing or improving cognitive performance having a single sensing and stimulating electrode set in accordance with some embodiments of the methods of the present application.

The system 120 includes the processor/controller(s) 14, the power source 3, the memory/data storage unit 16 which are interconnected as disclosed in detail hereinabove with respect to the system 10 of FIG. 1. The system 120 also includes a sensing and stimulating Electrode set 12E which may be used for both sensing electrical activity in the DLPFC 39 and for stimulating the DLPFC 39. The sensing and stimulating electrode set 12E may be implemented as a single implantable Ecog electrode array which is disposed on the surface of the DLPFC. The sensing in the DLPFC 39 is performed using standard sensing and/or recording methods as is known in the art of Ecog arrays. Stimulating of the DLPFC 39 may be performed by delivering stimulating current pulses or pulse trains through one or more electrode pairs of the multiple electrodes of the Ecog electrode array. The electronic/electrical circuits required for delivering stimulating electrical currents to the electrodes of the Ecog array (such as, for example, stimulus generating circuits, electrode multiplexing circuitry, timing circuitry, and any other required circuitry) are not shown in detail for the sake of clarity of illustration and are included in the controller circuitry of the processor/controller(s) 14.

When the system 120 is being used for augmenting and/or enhancing and/or/improving cognitive performance of a user, the electrode set 12E may be used to sense electrical activity in the DLPFC, process the data as disclosed in any of the methods disclosed in the present application (such as, for example, the methods illustrated in FIGS. 8, 9, 16 and 17) and process the signals sensed by suitable software operating on the processor/controller 14 to detect an indication that the user has been presented with a cognitive task or an intention of the user to perform a cognitive task. The indication may be any type of computable indication and/or neuronal activity pattern disclosed in the present application. If an indication has been detected, the processor/controller(s) 14 may stimulates the DLFPC by delivering electrical stimuli to the DLPFC (or part(s) of the DLPFC) through the electrode set 12E to augment and/or enhance and/or improve the cognitive performance of the user. In some embodiment the processor/controller(s) may (optionally) stop the sensing during the time period of stimulation of the DLPFC) and renew the sensing after the stimulation period is completed.

Reference is now made to FIG. 11 which is a schematic block diagram illustrating a system for augmenting or enhancing or improving cognitive performance having sensing and stimulating electrode set(s) for sensing in two cortical regions and for stimulating one or more cortical regions or one or more deep brain structures or a combination of one or more cortical regions and one or more deep brain structures, in accordance with some embodiments of the systems of the present application.

The system 130 is similar to the system 120 of FIG. 10 except that the Sensing/stimulating electrode set 12F is disposed on the prefrontal cortex (PFC) and on the temporoparietal cortex (TPC). The electrode set 12F may be implemented as a first Ecog electrode array disposed on the PFC and a second Ecog electrode array disposed on the TPC. The first Ecog electrode array may be used for sensing and for stimulating the PFC and the second Ecog electrode array may be used for sensing and for stimulating the TPC.

Alternatively, the electrode set 12F may be implemented as a single (possibly larger) Ecog electrode array disposed on both the PFC the TPC and capable of sensing and stimulating in both the PFC and the TPC. In operation, the system 130 may be operated in accordance with any of the methods disclosed herein (such as, for example, any of the methods disclosed in FIGS. 8, 9, and 16-19). The sensing may be performed in both the PFC and the TPC. The stimulation may be performed in either the PFC or the TPC but may also be performed in both the PFC and the TPC, resulting in enhancing and/or augmenting and/or improving the cognitive performance of the user.

It is noted that the stimulation methods used by the systems of the present application are not limited to stimulating cortical regions by electrodes or electrode set(s) implanted in the cortical regions (such as in Utah arrays), or by electrodes of subdural or epidural implanted Ecog electrode arrays or to stimulating deep brain structures by using DBS electrodes or electrode arrays implanted within or in the vicinity of the deep brain structure being stimulated. Rather, other stimulation methods may also be used as disclosed in detail hereinafter.

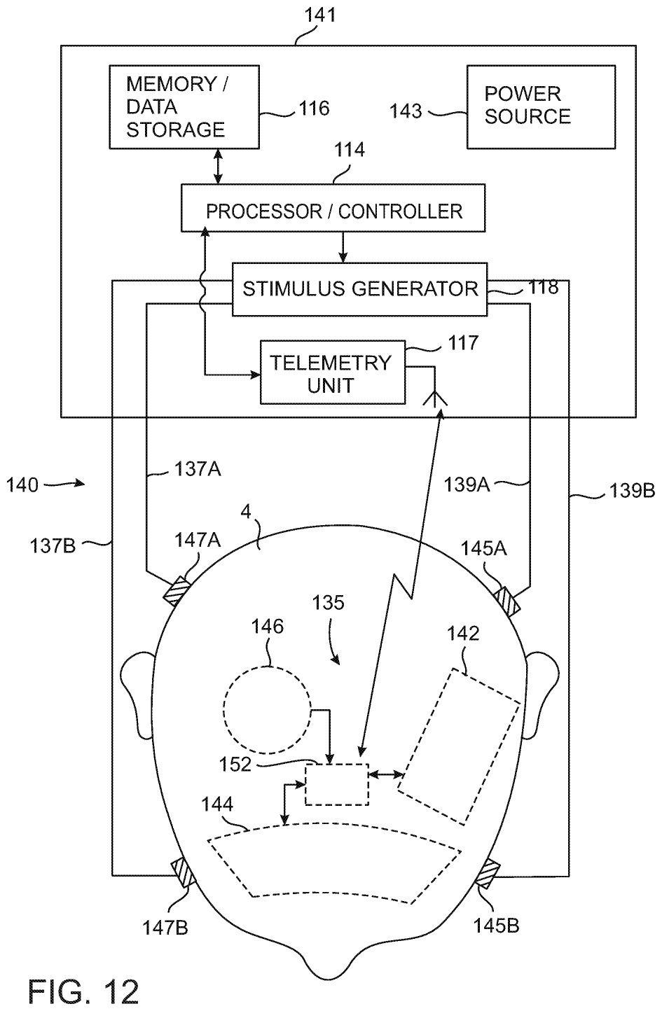

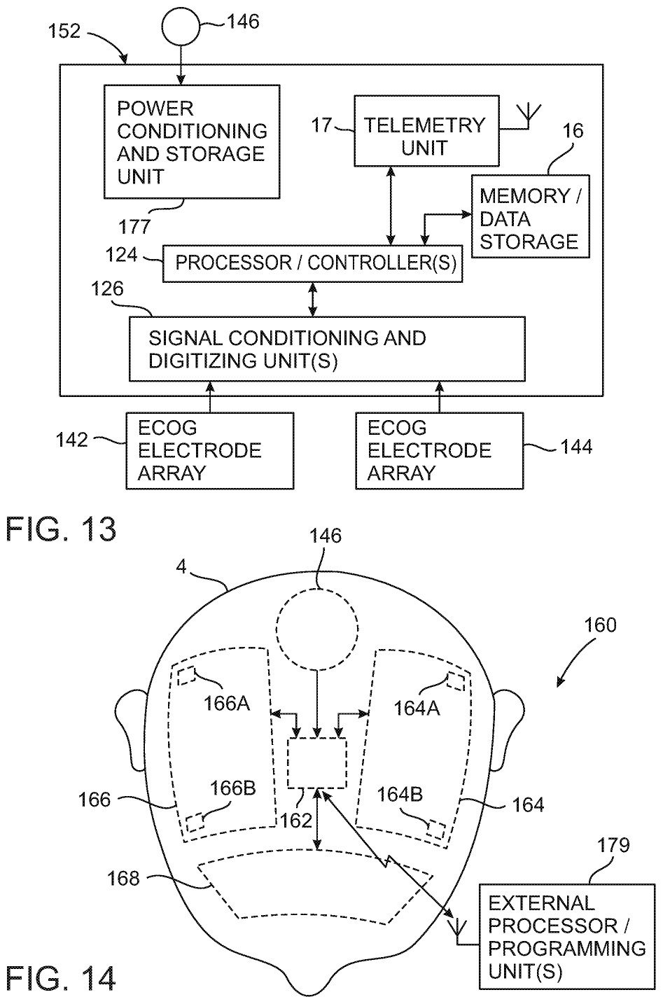

Reference is now made to FIGS. 12-13. FIG. 12 is a schematic block diagram illustrating a system for augmenting or enhancing or improving cognitive performance, including a set of non invasive electrodes for performing transcranial frequency interference stimulation of deep brain structures and intracranially implanted Ecog electrode arrays for sensing and/or stimulating one or more cortical regions, in accordance with some embodiments of the systems of the present application. FIG. 13 is a schematic block diagram illustrating the functional components of an intracranial part of the system of FIG. 12.

Turning to FIG. 12, the system 140 includes an extracranial module 141 and an intracranial module 135 wirelessly in communication with each other. The extracranial module 141 also includes one or more processor/controller(s) 114 suitably coupled to a memory/data storage device 116. The extracranial module 141 also includes a power source 143 for energizing the components of the extra cranial module 141. The stimulus generator 118 is suitably electrically connected to four stimulating electrodes 145A, 145B, 147A and 147B that are attached to the surface of the skin of the head 4 of the user at four different positions. The stimulating electrodes 145A, 145B, 147A and 147B may be electrically coupled to the surface of the skin of the head 4 by using any suitable electrically conducting gel or paste (such as for example any EEG electrode gel or paste). The stimulating electrodes 145A, 145B, 147A and 147B are connected to the stimulus generator 118 by suitable electrically conducting insulated leads 139A, 139B, 137A and 137B, respectively. A first stimulating current at a first frequency f may be applied by the stimulus generator 118 to a first electrode pair 145A and 145B and a second stimulating current at a second frequency f+Δf may be applied by the stimulus generator 118 to a second electrode pair 147A and 147B. Both frequencies f and f+Δf are in a frequency range too high to recruit neural firings (for example f and f+Δf≥1Khz). The stimulus generator 118 is suitably electrically connected to the processor/controller(s) 114 which controls the operation of the stimulus generator 118.

Due to the interference of the two different oscillating the electrical fields generated by the simultaneous stimulation through the first electrode pair 145A and 145B and the second electrode pair 147A and 147B at two different frequencies, selective neuronal activation may be achieved in deep brain structures that are located in a defined region where interference between the electric fields results in a prominent electrical field envelope modulated at the difference frequency Δf.

This selective stimulation method is referred to as temporal interference (TI) stimulation and is described in detail in the paper by Nir Grossman et al. referenced hereinabove and will also be interchangeably referred to as Non-invasive Temporal interference stimulation (NTIS) throughout the present application. The exact positioning of the electrodes on the head 4 of the user or patient and the stimulating intensity and frequencies may be determined, inter alia, by the position in the brain of the deep brain structure(s) that are being stimulated, the thickness and other physical and electrical parameters of the skull bones (which may significantly vary between different users of different ages) and may be empirically experimentally determined by suitable testing of each individual user/patient.

As the size and shape of the region of neuronal recruitment region in NTIS may be varied by adjusting or varying the positions of the stimulating electrodes 145A, 145B, 147A and 147B, and/or the stimulus frequency and intensity (amplitude) parameters, it is possible to stimulate one deep brain structure or several deep brain structures by suitably varying the size, shape and position of the neuronal recruitment region as disclosed in detail by Grossman et al.

The extracranial module 141 also includes a telemetry unit 117 suitably connected to the processor/controller(s) 114 for bidirectionally communicating with the intracranial module 135. The extracranial module 141 and the intracranial module 135 may telemetrically exchange data, control signals and status signals there between.

The intracranial module 135 may include an intracranially implanted electronic circuitry module 152, two Ecog electrode arrays 144 and 146 suitably electrically connected to the electronic circuitry module 152 and an intracranial induction coil 146 suitably electrically coupled to the electronic circuitry module 152 to provide electrical power to the electronics circuitry module 152 as is disclosed in more detail hereinafter. The Ecog array 144 may be disposed on the PFC preferably but not obligatorily, of both left and right cortical hemispheres (the cortical hemispheres are not shown in detail in FIG. 12, for the sake of clarity of illustration). The Ecog array 142 may be disposed on the left cortical hemisphere TPC as illustrated in FIG. 12.

Turning to FIG. 13, the electronics circuitry module 152 includes one or more processor/controller(s) 124, a power conditioning and storage unit 152, electrically coupled to the intracranial induction coil 146, a telemetry unit 17 suitably electrically coupled to the processor/controller(s) 124, a memory/data storage unit 16 suitably electrically connected to the processor/controller(s) 124 and a signal conditioning and digitizing unit(s) 126 electrically connected to the Ecog arrays 142 and 144 to receive sensed signals from the electrodes of the Ecog arrays 142 and 144. The conditioning and digitizing unit(s) 126 is also connected to the processor/controller(s) 126 for providing digitized sensed Ecog signal's data to the processor/controller(s) 126.

The Telemetry unit 17 may bidirectionally communicate with the telemetry unit 117 of the extracranial module 141, enabling bidirectional wireless transfer of data, control signals and status signals between the processor/controller 114 and the processor controller(s) 124.

It is noted that the power conditioning and storage unit 177 may include suitable circuitry (not shown in detail in FIG. 12 for conditioning electrical currents induced in the intracranial induction coil 146 by an extracranially placed second induction coil (not shown in FIGS. 12-13, for the sake of clarity of illustration) that may be placed on the scalp of the head 4 of the user. Alternating currents passing within such an extracranially placed second induction coil induce alternating currents within the intracranial first induction coil. The alternating currents flowing within the intracranial induction coil 146 may be rectified by suitable current rectifying diode bridge circuitry (not shown) included in the power conditioning and storage unit 177 and may be stored by any suitable charge storage device (not shown) such as, for example, a super-capacitor, a capacitor, or a rechargeable electrochemical cell included within the power conditioning and storage unit 177. The power conditioning and storage unit 177 is used for energizing any of the current requiring electrical components of the electronic circuitry module 152. It is noted that the electrical connections supplying electrical power to the components of the electronic circuitry module 152 are not shown in FIGS. 12-13 for the sake of clarity of illustration.

In operation, the system 140 may use any of the methods disclosed in the present application for modulating (i.e., enhancing and/or augmenting and/or improving) the cognitive performance of the user/patient. For example, the Ecog arrays 142 and 144 may sense signals from the TPC and PFC, respectively, the sensed signals may be conditioned (amplified and /or filtered) and digitized by the signal conditioning and digitizing unit(s) and fed to the processor/controller(s) 124 for processing (according to any of the processing methods disclosed in the present application. If the processor/controller(s) 124 detects an indication that the user has been presented with a cognitive task or intends to perform a cognitive task or performs a cognitive task, the system 140 may use the extracranial module 141 to stimulate a one or more deep brain structures by using the NTIS method as disclosed hereinabove using the electrodes 145A, 145B, 147A and 147B and the stimulus generator 118. Any of the deep brain structure(s) disclosed in the present application may then be stimulated using the extracranial module 141 to modulate the cognitive performance of the user/patient.

While the system 140 uses NTIS for non-invasively stimulating one or more deep brain structures and one or more invasive electrode sets, such as, for example the Ecog electrode arrays 142 and 144 (or other types of electrode arrays such as, for example UTAH electrode arrays with electrodes that may penetrate the surface of the cortex), this exemplary configuration is not obligatory to practice the methods disclosed herein. While the non-invasiveness of the stimulating electrodes in NTIS simplifies the stimulation procedure, the user has to be tethered to the extracranial module 141 (in cases where the module 141 is a large static module) or may have to carry (or wear the module 141 (in cases in which the module 141 is implemented as a small lightweight module that can be carried by the user). Additionally, using extracranial electrodes to perform NTIS may be inconvenient to the user, may be visibly unaesthetic and may also require frequent maintenance and care to avoid inadvertent electrode movements or undesirable variations in the electrical coupling characteristics of such extracranial stimulating electrodes to the skin.

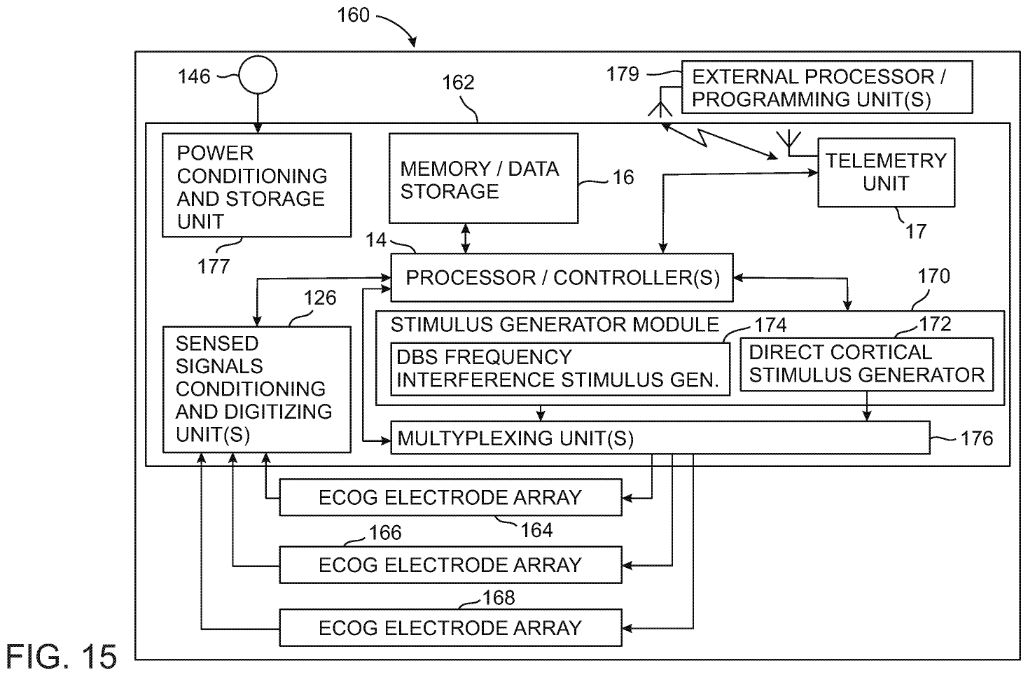

Reference is now made to FIGS. 14 and 15. FIG. 14 is a schematic drawing illustrating a system for augmenting or enhancing or improving cognitive performance, having multiple intracranial Ecog arrays for performing sensing in multiple cortical regions and for performing intracranial frequency interference stimulation of one or more deep brain structures and/or for directly stimulating one or more cortical regions, in accordance with some embodiments of the systems of the present application. FIG. 15 is a schematic functional block diagram illustrating functional components included in the system of FIG. 14.

Turning to FIG. 14, all of the components of the system 160 are intracranially disposed except for the external processor/programming unit 179 which is disposed outside the user). The system 160 includes an intracranially implanted electronics module 162, three intracranially implanted Ecog electrode arrays 164, 166 and 168 electrically connected to the electronics module 162, and an intracranial induction coil 146 electrically connected to the electronics module 162. The Ecog electrode array 168 may be disposed on the PFC or on a part or portion of the PLC. In accordance with some embodiments of the system 160, the Ecog electrode array 168 may be disposed on the PFC regions of both cortical hemispheres as illustrated in FIG. 14. Alternatively, in accordance with other embodiments of the system 160, the Ecog electrode array 168 may be disposed on the PFC or part thereof in the right cortical hemisphere. Alternatively, in accordance with other embodiments of the system 160, the Ecog electrode array 168 may be disposed on the PFC or part thereof in the left cortical hemisphere.

The Ecog electrode array 164 may be disposed on the TPC of the left cortical hemisphere or on a part of the TPC of the left cortical hemisphere. The Ecog electrode array 166 may be disposed on the TPC of the right cortical hemisphere or on a part of the TPC of the right cortical hemisphere.

Turning to FIG. 15, the system 160 may include one or more processor/controller(s) 14, a memory/data storage 16 suitably connected to the processor/controller(s) 14, a telemetry unit 17 suitably connected to the processor/controller(s) 14 for wirelessly transmitting data and/or control signals to an external processor/programming unit(s) 179 (which is disposed outside the body of the user). The system 160 may also include a power conditioning and storage unit 177 that is suitably electrically connected to the intracranial induction coil 146 to receive alternating currents therefrom. The structure and operation of the power conditioning and storage unit 177 is as disclosed hereinabove in detail with respect to the power conditioning and storage unit 177 of FIG. 13.

The system 160 may also include a stimulus generating module 170, suitably connected to and controlled by the processor/controller(s) 14. The stimulus generating module 170 includes a direct cortical stimulus generator 172 and a DBS Frequency Interference Stimulus Generator 174. The system 160 may also include one or more Multiplexing units 176. The multiplexing unit(s) 176 is/are suitably connected to the stimulus generator module 170 and to the processor/controller(s) 14 for controlling the delivery of stimuli from the DBS frequency stimulus generator 174 and from the direct cortical stimulus generator 172 to selected electrodes of the Ecog electrode arrays 164, 166 and 168.

The system 160 may also include one or more sensed signals conditioning and digitizing units 126 suitably electrically connected to the Ecog sensor arrays 164, 166 and 168 for conditioning the signals received from the electrodes included in the Ecog Arrays 164, 166 and 168 as disclosed in detail hereinabove with respect to FIG. 13.

The power conditioning and storage unit 177 may provide power for the operation of the electronics module 162. However, the connections providing power to the various components of the electronics module 162 are not shown in detail in FIG. 15 for the sake of clarity of illustration.

The external processor/programming unit(s) 179 may be any suitable processing device capable of telemetrically communicating with the Telemetry unit 17 of the electronics module 162. The processing device may be a computer equipped with Telemetry capabilities of communication (such as, for example WiFi) or any other hand held or portable device including processing and controlling and wireless communication components. For example, the external processor/programming unit(s) 179 may be a mobile or cellular telephone device or a Smartphone operating an application program that may telemetrically communicate with the telemetry unit 17 to control the operation of the electronics module 162, to receive and store data and status signals from the electronics module 162 and to display such data and status signals to the user of the system 160 (and/or to a physician or technician monitoring the patient or the user using the system 160), and to enable the user to send control signal for controlling the operation of the electronics module 162.

In operation, the system may then be conditioned 160 may sense electrical signals from one or more cortical regions of the user by using one or more of the Ecog electrode arrays 164, 166 and 168 (such as, for example, sensing in the PFC and/or the left TPC and/or the right TPC of the user). The sensed signals may be then conditioned (such, as for example, amplified and (optionally) filtered and then digitized by the sensed signals conditioning and digitizing unit(s) 126 and fed to the processor/controller(s) 14 for processing (according to any of the processing methods disclosed in the present application). If the processor/controller(s) 14 detects an indication that the user has been presented with a cognitive task or intends to perform a cognitive task or performs a cognitive task, the processor/controller(s) 14 may control the stimulus generator module 170 to stimulate one or more deep brain as follows. The processor/controller unit(s) 14 may control the multiplexing unit(s) 176 to select two spaced apart electrodes 164A and 164B of the Ecog electrode array 164 and two spaced apart electrodes 166A and 166B from the Ecog electrode array 166. After the electrodes have been selected, the processor/controller (s) 14 controls the DBS frequency interference stimulus generator 174 to apply an oscillating current or voltage having an oscillation frequency f between the electrode pair 164A and 164B and to simultaneously apply an oscillating current or voltage signal having an oscillation frequency of f+Δf. The two frequencies f and f+Δf may be larger or equal than 1 KHz. This temporal interference method of stimulation is somewhat similar but not identical to the NTIS method of Nir Grossman et al., as described hereinabove but differs from the NTIS method is certain aspects. A first difference between the two methods is that while NTIS uses extracranial non-invasive stimulating electrodes to achieve non-invasive deep brain stimulation while the other method described herein (with respect to the system 160 uses intracranial stimulating electrodes (of intracranially implanted Ecog electrode arrays or other intracranial electrode arrays) for stimulating one or more deep brain structures. To clearly distinguish the method using intracranial stimulating electrodes disclosed herein from the NTIS method, we refer to the second method throughout the present application as intracranial temporal interference stimulation (ICTIS).

Another advantageous difference between NTIS and ICTIS is that while in NTIS the extracranial electrodes stay fixed at the same place on the head, the stimulating electrodes used may be changed very fast by simply controlling the multiplexing unit(s) 176 to select different electrode pairs from any of the Ecog electrode arrays as the stimulating electrode pairs and deliver the two different interfering oscillation frequencies to any desired configuration of stimulating electrode pairs. This advantage may enable improved control of the modulation of the size, shape and location of the neuronal recruiting focal region formed within the brain.

Furthermore, the configuration of the system 160 allows additional control of the stimulation because the stimulation electrodes may be varied almost instantly by passing the oscillating stimulation signals through any selected combination of spaced apart electrode groups by applying the stimulating oscillation with frequency f to a pair of two different electrode groups having any desired electrode number and electrode configuration of the Ecog electrode array 164 array and simultaneously applying the stimulating oscillation with frequency f+Δf. To another different pair of two different electrode groups having any desired electrode number and electrode configuration selected from the Ecog electrode array 166. This electrode grouping variation method within each pair of stimulating electrode may allow much finer control of the parameters of the neuronal recruiting envelope region in comparison to the NTIS method which features static fixed sized stimulation electrode pairs.

Moreover, another advantage of the ICTIS method is that the configuration and positions of the electrode group pairs or of the pairs of single electrodes may be rapidly alternated between differently positioned stimulating group pairs or between differently positioned single electrode pairs allowing rapid alternating changing of the position and/or size and/or shape of the neuronal recruiting region, that may result is alternating stimulation of differently positioned deep brain structures within the brain of the user. This variation may also be useful for achieving finer temporal control of the deep brain structure if necessary (this means that it may be possible to stimulate different deep brain structures at different times following the detection of the indication disclosed hereinabove.

Another feature of the system 160 is that it may allow not only the stimulation of deep brain structures by NTIS or by ICTIS but may also allow the stimulation of selected regions of some cortical regions by directly applying stimulating signals (such as, for example, pulses or stimulating pulse trains) to any selected electrodes (or electrode pairs, or electrode groups). For example, the processor/controller(s) 14 may control the multiplexing unit(s) 176 and the direct cortical stimulus generator 172 to deliver direct stimuli to the TPC or to any part thereof through the electrodes of the Ecog electrode arrays 164 and 166, and/or to the PFC or any part thereof through the electrodes of the Ecog electrode array 168, or to any selected combinations of the PFC and TPC or portions thereof.

Furthermore, by using suitable multiplexing control, it may be possible to perform several types of stimulation regimes including, for example, simultaneous stimulation of one or more deep brain structures and one or more cortical regions, simultaneous stimulation of one or more cortical regions (for example, the PFC and TPC), stimulation of a single deep brain structure (by ICTIS), stimulation of a single cortical region or a part thereof by direct stimulation through a selected one of the Ecog electrode arrays 164, 166 and 168. Any combinations and permutation of such stimulation regimes/methods may be performed.

Specific Exemplary Methods of Using the BCI Systems

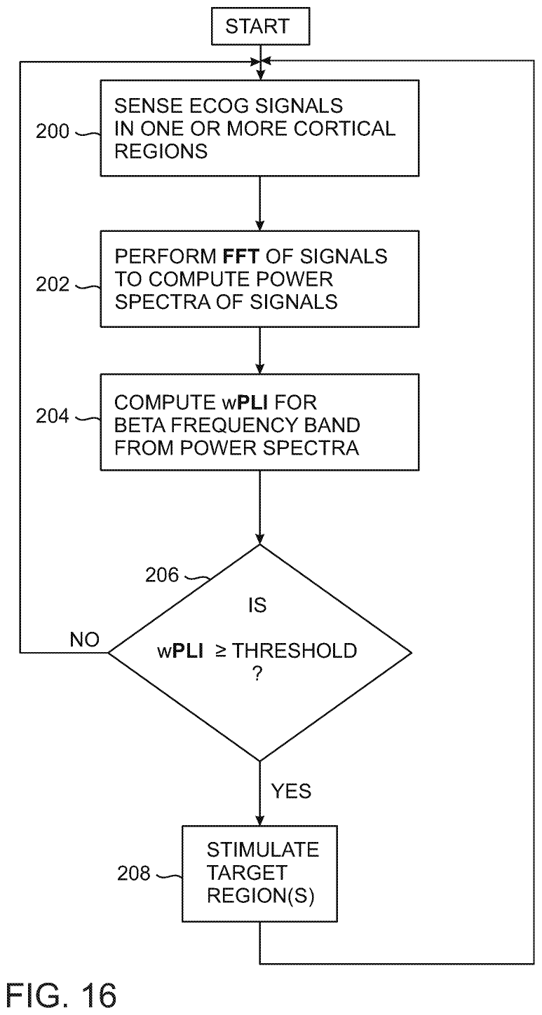

Reference is now made to FIGS. 16-19 which are a schematic flow chart diagrams illustrating steps of four different exemplary methods for augmenting or enhancing or improving cognitive performance of a user, in accordance with some embodiments of the methods of the present application.

It is noted that these exemplary methods may be performed by suitable software programs operating on any of the processor/controller(s) of the various systems disclosed hereinabove.

Turning to FIG. 16, the method includes the step of sensing Ecog signals in one or more cortical regions of the user (step 200). The sensed signals may be recorded and/or stored (in a digitized form) in the memory/data storage unit(s) of the system (such as, for example in the memory/data storage 16 or 16 disclosed hereinabove). The method may then process the sensed digitized signals or the stored or data by performing a Fourier transform (FT) on the data (such as for example a fast Fourier transform algorithm) to compute power spectra of the digitized data of the sensed signals (step 202). The method then uses the power spectra to compute a value of the weighted phase lag index (wPLI) at the beta frequency band (the band having the frequency range of 15-30 Hz) as is disclosed in detail hereinafter (step 204). The method then compares the computed value of the wPLI to a threshold value (step 206). If the value of wPLI is greater or equal to the threshold value, the method stimulates one or more target regions (step 208) and transfers control to step 200.

If the value of wPLI is smaller than the value of the threshold, the method transfers control to step 200 to continue the sensing of the Ecog signals.

The target regions of step 208 may be either one or more cortical regions, or one or more deep brain structures, or a combination of one or more cortical regions and one or more deep brain structures. Deep brain structures that may be stimulated in step 208 may include, the ventral tegmental area (VTA), the striatum, the caudate nucleus, the putamen, the nucleus accumbens (NA), the locus ceruleus, the hippocampus, the amygdala, a deep brain structure of the meso-limbic system, a deep brain structure functionally participating in enhancing and/or facilitating learning, memory and attention focusing, a subcortical region of the brain, the substantia nigra, a subcortical region of the brain, the dorsal striatum, a part of the limbic structures within the mesocortical system, a part of the nigrostriatal system, a part of the tuberoinfundibular system, the fornix, the nucleus basalis of meynert (NBM), the anterior caudate nucleus, the dorsal striatum, the anterior thalamic nucleus, the central thalamus, the lateral hypothalamus, the subgenual cingulated region (BA 25), the enthorinal cortex, the peforant path, the medial frontal lobe, the subthalamic nucleus and any combinations thereof.

In patients suffering from depression, some of the preferred deep brain targets may include but are not limited to, the subgenual cingulated region (BA 25), the ventral capsule (VC)/ventral striatum (VS), the NA. The lateral Habenula, the ventral caudate nucleus and the inferior thalamic peduncle.

In normal users some of the preferred deep brain targets may include but are not limited to, the ventral tegmental area (VTA), the striatum, the caudate nucleus, the putamen, the nucleus accumbens (NA), the locus ceruleus, the hippocampus, the amygdala, a deep brain structure of the meso-limbic system, a deep brain structure functionally participating in enhancing and/or facilitating learning, memory and attention focusing, a subcortical region of the brain, the substantia nigra, a subcortical region of the brain, the dorsal striatum, a part of the limbic structures within the mesocortical system, a part of the nigrostriatal system, a part of the tuberoinfundibular system, the fornix, the nucleus basalis of Meynert (NBM), the anterior caudate nucleus, the dorsal striatum, the anterior thalamic nucleus, the central thalamus, the lateral hypothalamus, the enthorinal cortex, the peforant path, the medial frontal lobe, the subthalamic nucleus and any combinations thereof.

If the target brain regions comprise one or more cortical regions, the stimulation of step 208 may be performed in one or more of, the prefrontal cortex (PFC), a part of the PFC, the dorsolateral prefrontal cortex (DLPFC), a part of the DLPFC, the temporoparietal cortex (TPC), a part of the TPC, the inferior frontal gyrus (IFG), a part of the IFG, the temporal parietal junction (TPJ), a part of the TPJ, and any combinations thereof.

It is noted that as disclosed hereinabove, any combination of the above cortical may be stimulated in step 208.

It is noted that some variations are possible in the method disclosed in FIG. 16. For example, while the stimulating of the target brain regions may be performed right after the detection of the indication of step 206, in some embodiments, the stimulation may be performed after a suitable delay period, depending, inter alia on the specific target brain regions being stimulated in step 208.

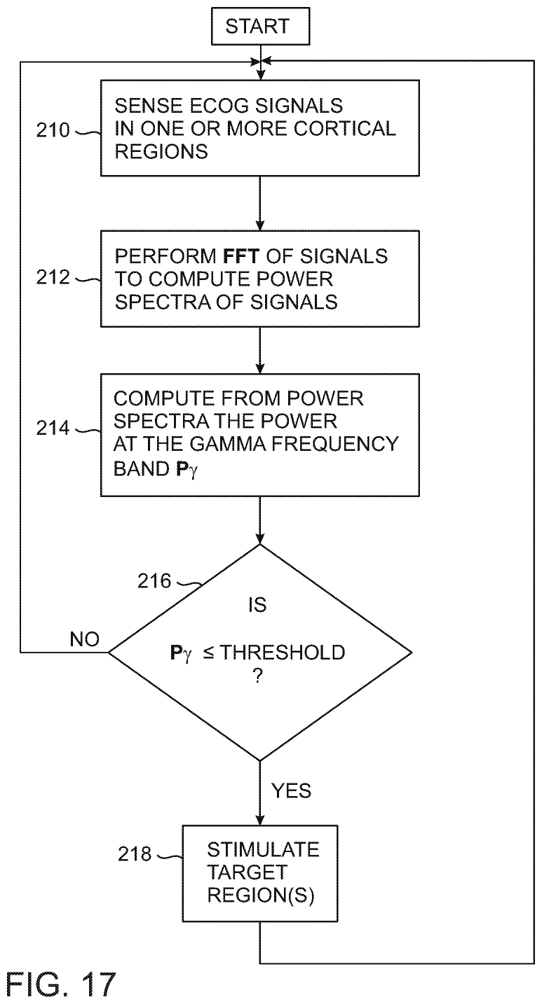

Turning to FIG. 17, the exemplary method illustrated in FIG. 17 includes sensing Ecog signals in one or more cortical regions (step 210). A Fourier transform, such as for example a fast Fourier transform (FFT) is performed on the sensed (and recorded and digitized) signals to compute the power spectra of the signals (step 212). The method then computes from all the power spectra the momentary power Pγ at the gamma frequency band (step 214). The momentary power Pγ represents a power value computed for small blocks of time (for example, about 1 second) from the power spectra of a selected number of electrodes. The gamma band is the band including the frequency range f≥30 Hz). The method then compares the computed value of Pγ with a threshold value (step 216). If the value of Pγ is smaller than or equal to the threshold, the method stimulates one or more brain target regions (Step 218) and transfers control to step 210 to continue the sensing. If the value of Pγ is larger than the threshold value, the method returns control to step 210 to continue the sensing. The cortical region(s) in which sensing is performed by step 210 may be similar to the cortical region(s) in which the sensing of step 200 of FIG. 16.

The target brain region(s) that may be stimulated in step 218 may be similar to the target brain region(s) of step 208 of FIG. 16.

In some embodiments of the methods, the sensing in the cortical target region(s) may be stopped while the stimulation of the target brain region(s) is being stimulated (possibly in order to avoid sensing stimulation artifacts that may be induced by the stimulation).

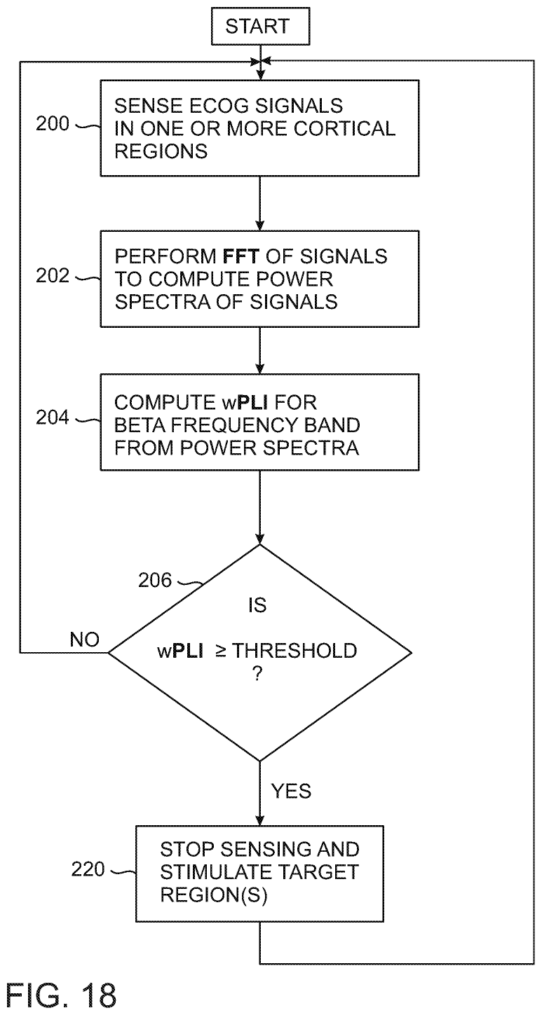

Turning to FIG. 18, the method disclosed in FIG. 18 may be similar to the method illustrated in FIG. 16, with the exception that in step 220 of the method of FIG. 18, the sensing in the cortical region(s) is stopped for the duration of the stimulation of the target brain region(s) and is continued after control is transferred to step 200 of FIG. 18.

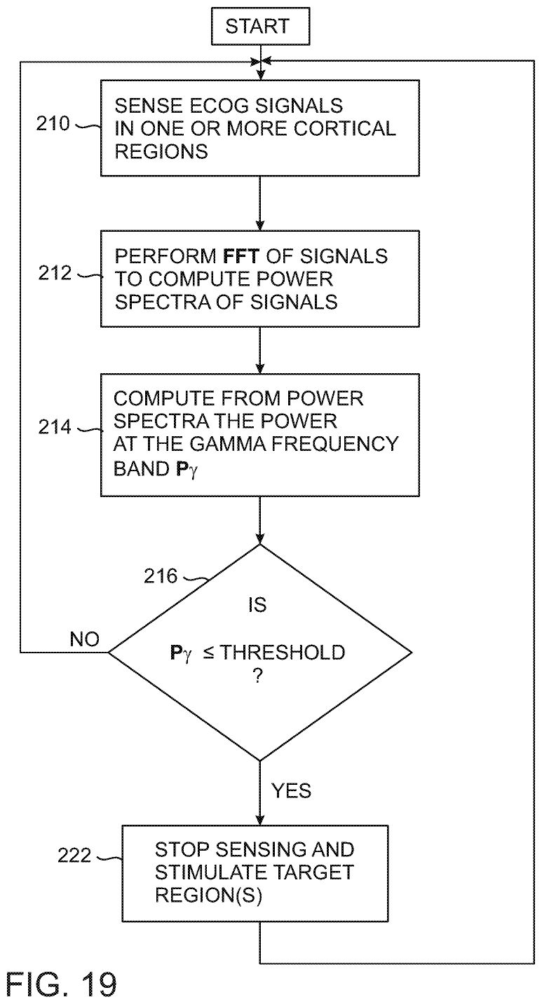

Turning to FIG. 19, the method disclosed in FIG. 19 may be similar to the method illustrated in FIG. 17, with the exception that in step 222 of the method of FIG. 19, the sensing in the cortical region(s) is stopped for the duration of the stimulation of the target brain region(s) and is continued after control is transferred to step 210 of FIG. 19.

Other possible embodiments of the methods of FIGS. 18 and 19 may include modifying steps 220 and 220, respectively such that the stimulation of the target brain regions starts after a delay time period has passed from the time point of the detection of the indication in steps 206 and 216, respectively.

Method of Computation of wPLI

The method of computing the wPLI are known in the art and are disclosed in detail in the following papers:

Christiano Micheli, Daniel Kapinf, Stephanie Westendorff, Taufikand A. Valiante and Thilo Womelsdorf, entitled “ Inferior-Frontal cortex phase synchronizes with temporal-parietal Junction prior to successful change detection”, published in Neuroimage, 119, pp.417-431 (2015).

and

Martin Vinck, Robert Oostenveld, Marijnvan Wingerden, Franscesco Battaglia and Cyriel M. A. Pennartz, entitled “An improved index of phase-synchronization for electrophysiological data in the presence of volume-conduction, noise and sample-size bias”, published in Neurolmage, 55, pp. 1548-1565, (2011).

Briefly, spectral analyses are performed by Fourier analysis applied to the same size, 0.5 s time windows. Data are tapered using tapers discrete dpss (prolate spheroidal sequences) and +/−4 Hz frequency bandwidth (corresponding to 3 tapers). For inspection and visualization of examples, a time frequency analysis is run using 0.5 s windows whose centers were 0.05 s distant from the next/previous window, allowing for a 0.45 s overlap.

Analysis of Phase Synchronization

To study connectivity between signals from separate electrodes, we compute the weighted phase lag index (wPLI). The wPLI is a measure of phase synchronization (similar to coherence) that is based solely on the imaginary component of the cross-spectrum, and is not spuriously affected by the volume conduction of a single source's activity to two separate sensors (such as, for example two sensing electrodes). The wPLI is monotonically related to increases in true phase-coupling between interacting sources. An advantage of the wPLI is that it is invariant to a linear mixing of two dependent sources, and is sensitive in detecting interactions when the interacting sources are spatially close. A direct estimator of the wPLI is biased by sample size. We therefore estimated the squared wPLI by using the debiased wPLI estimator ranging from zero (negative values can incidentally occur because of limited sampling) to one (maximum phase synchronization). The debiased wPLI has no sample size bias if the asymptotic wPLI value equals zero (no phase coupling), hence the debiased wPLI does not spuriously indicate interactions. Furthermore, its sample size bias is negligible for even small sample sizes of 20-30 trials. Note that the debiased wPLI is an estimate of the squared wPLI, that is, a value of 0.1 for the debiased wPLI corresponds to a value of the unbiased wPLI of about 0.3. The wPLI considers the cross-spectrum between two channels (for example, two electrodes) and, for each frequency, it weights linearly the phase difference between 0 and 90° (at zero it is nulled, at 90° it is weighted as 1) the equation for calculating the value of the wPLI is:

$$wPLI = \frac{\vert E \{ \vert Imag ( NDC ) \vert sign ( Imag ( NDC ) ) \} \vert}{E \{ \vert Imag ( NDC ) \vert \}}$$

The cross-spectrum C(f)=X(f)Y*(f). The matrices X and Y are the FFT transforms of channel X and channel Y, respectively, * is the conjugate matrix operator and C is the cross-spectrum. The complex non-diagonal part of C is referred to as NDC (non-diagonal cross-spectrum), Imag(.) is the imaginary part operator, |.I is the absolute value operator and E{.} indicates the expected value operator (the sample mean) across trials. The dependency of NDC from frequency is omitted, although it is always implicitly assumed.

It is noted that the value of wPLI may be calculated from the power spectra set of an entire Ecog electrode array but, practically it may be calculated from the power spectra of a selected set of sensing electrodes (a subsample) for computational efficiency.

When the sensing with two Ecog electrode arrays each positioned at a different cortical region, for example, the PFC and the TPC, theoretically, given a sufficiently high computational power, it may be possible to compute the wPLI from the power spectra of all possible combinations of electrode pairs for which one electrode of the pair is sensing in the PFC and the other electrode of the pair is sensing in the TPC. However, practically, due to limited computational power, the computation of wPLI may be performed on a limited subset of such electrode pairs.

Methods for Computing Pγ

The power spectrum (Sxx,j) of a signal x is defined as follows:

Sxx,j=(2Δ²/T) Xj Xj *, which is the product of the Fourier transform of x at frequency fj (Xj) with its complex conjugate (Xj *), scaled by the sampling interval (A) squared and the total duration of the recording (T). Notice the units of the power spectrum are (in this case): (μV)²/Hz.

Data from the screening session may be analyzed offline by re-referencing cortical signals to the common average and using an autoregressive method for spectral power estimation known as the Maximum Entropy Method (MEM) to calculate spectral power in 1 Hz bins from 1-50 Hz using 500 msec sliding windows. Following the screening task, a single calibration run may be performed and serves to validate the chosen BCI control feature (the BCI control feature is a signal or indication of a physiological change that reflects a change in neurological status of the user or patient).

BCI Control Sessions

During online closed loop sessions, cortical signals are re-referenced to the common average and spectral analysis is performed in 1 Hz bins on 500 msec windows of cortical data shifted by 125 msec per window using the MEM algorithm. After each 500 msec window is collected, the spectral power at the control feature is used to update the stimulation regime as described by the following equation:

$$Y ( t ) = Y ( t - 1 ) + Gain \frac{( X ( t ) - {μ}_{Rest} - Bias )}{{σ}_{Rest}} sign ( {μ}_{active} - {μ}_{Rest} )$$

where Y(t) is the current cortical stimulation constrained to the 0-100% range of maximal cortical stimulation range, Y(t−1) is the previous stimulation setting, X(t) is the current value of and rest trials, σᵣₑₛₜ is the standard deviation of the BCI control feature during the rest trials, Gain is a gain term controlling the intensity of stimulation, and Bias is a bias term designed to improve the ability to discriminate rest periods.

It is noted that the methods disclosed hereinabove are not limited to the specific methods and algorithms indicated hereinabove. For example the methods may include computing of the power spectra and P (the momentary spectral power) and/or wPLI at any frequency band by any method of spectral analysis known in the art and is not limited to using Fourier transform methods such as FFT.

Additionally, while the specific exemplary methods disclosed hereinabove compute the value of P_{f} (the momentary power at ant selected frequency band f) as Pγ (the momentary spectral power in the gamma frequency band), this is not obligatory and the value of P_{f} may be computed and used (instead of Pγ) at any desired frequency band or bands such as, for example the delta, theta, mu, alpha, beta and gamma bands or for any selected combinations of these bands. Similarly, the method of using the detection of an alteration in the phase of the sensed signals is not limited to the computation of the parameter wPLI in the gamma frequency band, or to computing the wPLI at all, but may be also performed by any algorithm or method for detecting phase alterations in any of the frequency bands disclosed hereinabove (such as, for example the delta, theta, mu, alpha, beta and gamma bands or any selected combinations thereof). Any such methods for computing or detecting an alteration in phase or in spectral power at any of the above disclosed frequency bands may be used in the systems and methods disclosed herein.

The systems disclosed herein may also be used for enhancing human cognitive performance. In accordance with some embodiments of the methods of use of the systems, the systems may be used for treating cognitive deficits in human patients having a neurological impairment or neurological disorder or neuro-psychiatric disorder.

The BCI systems such as, for example, the systems 10, 30, 40 and 50, 60, 80, 120, 130, 140 and 160 may be used, inter alia, to improve the rate of learning (or relearning) tasks in individuals with brain injury, stroke dementia, neurodegenerative disorder, or other lesions that impede or adversely affect cognitive functions in such patients. Additionally, for individuals with stroke, dementia, neurodegenerative disorder, lesions in the PFC, or problems with impaired working memory or with impaired ability of maintaining sustained attention, the systems disclosed herein may be used to improve their working memory and sustained attention maintaining performance. The systems and methods disclosed in the present application may also be used for treating individuals with ADHD or ADD by modulating and/or controlling neuronal activity in brain regions associated with the cognitive task of attention focusing.

Furthermore, in accordance with some embodiments of the systems of the present application, the systems and methods disclosed in the present application may detect neuronal activity patterns associated with OCD type of behavior in the same or in other different brain regions and may also be used for treating neurological and/or neuro-psychiatric patients suffering from OCD, by suitably modulating neuronal activity in selected brain regions as disclosed by Nikolaos Makriset et al. In the paper entitled “Variability and anatomical specificity of the orbitofrontothalamic fibers of passage in the ventral capsule/ventral striatum (VC/VS): precision care for patient-specific tractography-guided targeting of deep brain stimulation (DBS) in obsessive compulsive disorder (OCD). ” published in Brain Imaging and Behavior, December 2016, Volume 10, Issue 4, Pp. 1054-1067.

Additionally or alternatively, the systems and methods disclosed herein may be used in normal users for enhancing and/or augmenting and/or improving cognitive performance such as, inter alia, the rate of learning, working memory (WM) and sustained attention.

The human striatum is a deep brain structure that is analogous to a “weighted learning engine”. During learning tasks, with deep brain electrical stimulation of the striatum, causes the VTA and deeper structures to release dopamine which reinforces connections between relevant neurons that are strengthened during that process of learning the new task. This results in doubling the rate of learning.

The dorsolateral prefrontal cortex (DLPFC) is a cortical surface region (BA 46 and 9) that controls working memory and sustained attention, some of the most critical components of executive cognition. It is possible to sense distinct patterns of neuronal activations within the DLPFC during tasks involving holding items in working memory and sustaining attention.

In the different systems disclosed in the present application, a deep brain stimulator/embedded mesh electronics/stent array, may be used to stimulate the striatum or other deep brain structures at specific times during working memory activation or sustaining attention, to reinforce and strengthen connectivity that is associated with “positive” cognitive behavior (such as storing several items within working memory and retrieving them correctly, or sustaining attention beyond a particular threshold).

It is noted that in all of the systems disclosed in the present application, the connections between the various components of the system may be implemented as wired connections or as wireless connections (by using appropriate wireless transmitters, and/or wireless receivers and/or wireless transceivers. Any suitable method of wireless transmitters may be used provided that they are operable in the conditions surrounding the systems, if the electrode set(s) and processor/controller are disposed on the surface of the brain or intra-cranially it may be possible to use radio frequency (RF) wireless systems, but other wireless communication methods and devices may be used as is known in the art, such as ultrasonic wireless communication devices which may be suitable for implanted brain devices), Infrared wireless methods and/or devices, optical wireless communication devices and methods and the like.

It is also noted that the type of wired or wireless communication links between various components of all the systems disclosed herein may be unidirectional (such as, for example, a link used only for sensing or only for stimulation) but may also be a bidirectional link for bidirectional communication. For example, the connection between a sensing electrode array (such as any of the sensing electrode sets disclosed herein) to the processor/controller may enable the delivery of stimulating signals through the sensing electrode set. This may be useful for testing the electrodes to determine any changes in the electrical properties of the electrodes and/or in the brain tissue in close contact with the electrodes or in the vicinity thereof. Similarly, the link to a stimulating electrode set may also be bidirectional link to enable sensing the electrical responses to test pulses for testing the electrical properties of the stimulating electrode(s) and their close environment. Other tests may be performed to test short term, medium term and long term changes in neuronal viability or activity and/or to monitor such changes with time.

It is noted, that in accordance with some embodiments of the systems and methods of the present application, enhancement and/or augmentation and/or improvement of the user's (or patient's) cognitive performance may be achieved without stimulating deep brain structures as disclosed hereinabove.

For example, in accordance with some embodiments of the systems of the present application, the system may include one or more electrode sets for sensing/recording in the DLPFC signals associated with task neuronal activity which are indicative of the performance of a cognitive task and/or of the intention to perform such a cognitive task as disclosed in detail hereinabove. However, in contrast to the previously disclosed exemplary systems which include one or more electrode sets for delivering stimulation to deep brain structures, in some embodiments of the systems, the system may include one or more electrode sets which are capable of delivering stimulation to the DLPFC upon detecting neuronal activity associated with an intention to perform a cognitive task and/or the presentation of a cognitive task.

For example, turning to FIG. 3, the system for sensing and stimulating in the DLPFC may include the processor/controller 14, the power source 3, the memory/data storage 16, the (optional) auxiliary sensor(s) 18, the (optional) effector device(s) and the sensing electrode set(s) 12C. (the stimulating electrode set(s) 12D is not included in such a system). The sensing electrode set(s) 12C and the processor/controller 14 may be used for sensing/recording in the DLPFC signals associated with neuronal activity, as disclosed in detail hereinabove, and for processing the sensed/recorded signals to detect a spatio-temporal neuronal activity pattern associated with or indicative of the performance of a cognitive task and/or the intention to perform such a cognitive task, as disclosed hereinabove in detail.

Once such a pattern is detected, the system may use the same sensing electrode set(s) 12C, for stimulating certain regions of the DLPFC. The DLPFC stimulation delivered to the DLPFC responsive to such a detection may result in local activation of dopaminergic synapses terminating on dendrites or cell bodies of neurons involved in localized DLPFC circuits which may result in reinforcement of such circuits leading to enhancement/augmentation/improvement of the cognitive performance of the user.

Such systems may be advantageous because they eliminate the need for performing the relatively more complex surgical procedures for implanting electrode set(s) capable of delivering stimulation of deep brain structures. Additionally, because both sensing/recording and stimulation of the DLPFC may be performed by a single electrode set, the disclosed system may include less components. In such systems, the sensing electrode set(s) 12C may be an Ecog type (of any known type) electrode array which may be in contact with the surface of the DLPFC or with the overlying pia or dura, but may also be one of the flexible mesh type electrode arrays disclosed in the papers by Lieber and coworkers above which may be implanted within the cortical tissue. Utah Arrays may also be used for sensing/stimulating the DLPFC by penetrating into the cortical tissues of the DLPFC. Stentrode arrays may also be employed for sensing and stimulating the DLPFC as disclosed in detail hereinabove.

It will be appreciated that while a single electrode set or electrode array (such as, for example the sensing electrode set(s) 12C) may be sufficient for both sensing and stimulating in the DLPFC, this is not obligatory for practicing this embodiment of the invention and more than one set of electrode set of electrode arrays may be used. Some embodiments may include one (or several) electrode set for sensing in the DLPFC and another (or several other) electrode sets for delivery of stimulation to the DLPFC. For example, an Ecog type electrode set may be used for sensing/recording and a n implanted flexible mesh type electrode set may be used for stimulation of the DLPFC. In some embodiments, an implanted mesh type flexible electrode array may be used for stimulation and a stentrode may be placed in a brain blood vessel of the DLPFC region for sensing. Any such suitable permutations and variations of electrode set types and numbers may be used to implement the system for sensing in and stimulating the DLPFC for augmenting/enhancing/improving cognitive performance disclosed herein.

Furthermore, such systems may be used in normal users as well as in patients having neurological and/or psychiatric and/or neuro-psychiatric disorders and/or disabilities as disclosed in detail hereinabove.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A brain computer interface (BCI) system for augmenting and/or assisting and/or improving cognitive performance of a user, the system comprising:
one or more electrode sets for sensing signals associated with neuronal electrical activity in one or more cortical regions of the user and for providing stimulating signals to one or more target brain regions;
at least one processor/controller in communication with the one or more electrode sets, the at least one processor/controller is programmed to process signals sensed in the one or more cortical regions for detecting an indication associated with an intention to perform a cognitive task and/or the presentation of a cognitive task and/or the performing of a cognitive task, and to control the stimulating of the one or more target brain regions responsive to the detecting of the indication for augmenting and/or assisting, and/or improving the cognitive performance of the user, and
at least one power source for energizing the BCI system.

2. The system according to claim 1, wherein the one or more target brain regions are selected from the group consisting of,
one or more deep brain structures of the user,
one or more cortical regions of the user, and
a combination of one or more cortical regions and one or more deep brain structures.

3. The system according to claim 1, wherein the one or more cortical regions comprise one or more of prefrontal cortex (PFC), a part of the PFC, a dorsolateral prefrontal cortex (DLPFC), a part of the DLPFC, a temporoparietal cortex (TPC), a part of the TPC, an inferior frontal gyrus (IFG), a part of the IFG, the temporal parietal junction (TPJ), a part of the TPJ, and any combinations thereof.

4. The system according to claim 2, wherein the one or more deep brain structures are selected from ventral tegmental area (VTA), striatum, caudate nucleus, putamen, nucleus accumbens (NA), locus ceruleus, hippocampus, amygdala, a deep brain structure of the meso-limbic system, a deep brain structure functionally participating in enhancing and/or facilitating learning, memory and attention focusing, a subcortical region of the brain, a substantia nigra, a dorsal striatum, a part of the limbic structures within a mesocortical system, a part of a nigrostriatal system, a part of teberoinfundibular system, fornix, nucleus basalis of Meynert (NBM), anterior caudate nucleus, dorsal striatum, anterior thalamic nucleus, central thalamus, lateral hypothalamus, subgenual cingulated region (BA 25), enthorinal cortex, perforant path, medial frontal lobe, subthalamic nucleus and any combinations thereof.

5. The system according to claim 1, wherein the cognitive performance comprises one or more of, attention focusing performance, memory performance, short term memory performance, learning performance, memory retrieval performance, working memory performance and any combinations thereof.

6. The system according to claim 1, wherein the cognitive task is selected from, an attention focusing task, an attention sustaining task, a memorizing task, a short term memory requiring task, a learning task, a memory retrieval task, and any combinations thereof.

7. The system according to claim 1, wherein the user is selected from a normal user and a user having a neurological disorder, a psychiatric disorder, or a neuro-psychiatric disorder.

8. The system according to claim 7, wherein the neurological disorder or psychiatric disorder or psychiatric-neurological disorder is selected from, ADHD, ADD, a learning deficiency, an attention related deficiency or dysfunction, amnesia, a memory related dysfunction, anxiety, depression, traumatic brain injury, stroke, dementia, neurodegenerative disorder and any combinations thereof.

9. The system according to claim 1, wherein the one or more electrode sets are configured for sensing neuronal electrical activity in one or more additional cortical regions of the user and/or for stimulating neurons in the one or more additional cortical regions selected from a visual cortical region, a region of the primary visual cortex (V1), the medial temporal lobe of the visual cortex, a region of the motor cortex, a region of the pre-motor cortex, a region of the somato-sensory cortex, a region of the auditory cortex, the mesial surface of the right cortical occipital lobe, the associative cortex, the primary visual cortex, other areas of the visual cortex, the auditory cortex, the motor cortex, BA 17, BA 18, BA 19, BA 7, BA 6, BA 5, BA 4 and any combinations thereof.

10. The system according to claim 1, wherein the one or more electrode sets are selected from, non-invasive electrode sets, invasive electrode sets, and any combinations thereof.

11. The system according to claim 1, wherein the one or more electrode sets is selected from,
at least one sensing and stimulating electrode set configured for performing sensing in the one or more cortical regions and for stimulating one or more of the target brain regions,
at least one sensing electrode set configured for performing sensing in the one or more cortical regions and at least one stimulating electrode set for stimulating one or more of the target brain regions,
at least one electrode set configured for performing sensing in one or more cortical regions and for stimulating at least one cortical region of the one or more cortical regions, and
at least one electrode set configured for sensing in the DLPFC and for stimulating the DLPFC.

12. The system according to claim 1, wherein the one or more electrode sets is selected from,
at least one electrode set configured for sensing signals associated with neuronal electrical activity in the one or more cortical regions and at least one electrode set configured for stimulating one or more deep brain structures by using temporally interfering (TI) electric fields, and
at least one electrode set configured for sensing signals associated with neuronal electrical activity in the one or more cortical regions and for stimulating one or more deep brain structures by using temporally interfering (TI) electric fields.

13. The system according to claim 1, wherein the one or more electrode sets are selected from, an electrode assembly comprising two or more electrodes, a multi-electrode array, an implantable electrode array, an injectable mesh electrode array, a multiplexable electrode array, a flexible electrode array, a flexible electrode array adapted to be applied on a cortical surface, a linear electrode array, an Ecog surface electrode array, a μEcog electrode array, an intra-cortically implantable electrode array, a stent electrode, a stent electrode array, neural dust sensing device(s), EEG electrodes, an electrode set including two or more electrodes implanted under the scalp, an electrode set configured for performing non-invasive transcranial frequency interference stimulation (NTIS), an electrode set configured for performing intracranial frequency interference stimulation (ICTIS) and any combinations thereof.

14. The system according to claim 1, wherein the signals associated with neuronal electrical activity are selected from, extracellularly recorded single neuron action potentials, extracellularly recorded electrical field potentials, and any combinations thereof.

15. The system according to claim 1, wherein the system also includes a telemetry unit in communication with the at least one processor/controller for wirelessly communicating with an external telemetry unit.

16. The system according to claim 1, wherein the at least one processor/controller is selected from, at least one processor/controller external to the cranium of the user, at least one intracranial processor/controller, at least one wearable processor controller, at least one remote processor/controller, at least one digital signal processor (DSP), at least one graphic processing unit (GPU), at least one quantum computing device (QCD), a quantum computer and any combinations thereof.

17. The system according to claim 1, wherein the indication is selected from,
an alteration in a computed weighted phase lag index (wPLI) in the beta frequency band, an alteration in computed spectral power (Pγ) in the gamma frequency band, and
an alteration in the computed wPLI in the beta frequency band of cortical electrical activity sensed in one or more electrode pairs at the beta frequency band and an alteration in spectral power at the gamma frequency band.

18. The system according to claim 1, wherein the at least one power source is selected from, at least one power source external to the cranium of the user, at least one intracranial power source, at least one wearable power source, at least one intracranial power receiver for wirelessly receiving power from an extracranial power source, at least one intracranial power receiver for wirelessly receiving and storing power from an extracranial power source, at least one intracranially implanted induction coil adapted for receiving electrical power from an extracranially disposed induction coil, and any combinations thereof.

19. A method for enhancing and/or assisting and/or improving cognitive performance of a user, the method comprising the steps of:
sensing signals associated with neuronal activity in one or more cortical regions;
processing the signals for detecting an indication associated with an intention to perform a cognitive task and/or the presentation of a cognitive task and/or the performing of a cognitive task; and
stimulating one or more target brain regions of the user responsive to the detecting of the indication for enhancing and/or improving and/or assisting the cognitive performance of the user.

20. The method according to claim 19, wherein the one or more target brain regions are selected from,
one or more deep brain structures,
one or more cortical regions, and
a combination of one or more deep brain structures and one or more cortical regions.

21. The method according to claim 19, wherein the user is selected from a normal user and a user having a neurological disorder, and/or a psychiatric disorder, and/or a neuro-psychiatric disorder.

22. The method according to claim 21, wherein the user is a user having a neurological disorder, and/or a psychiatric disorder and/or a neuro-psychiatric disorder, and wherein the step of stimulating improves the cognitive performance of the user as compared to the cognitive performance of the user when the step of stimulating is not performed.

23. The method according to claim 21, wherein the neurological disorder and/or the psychiatric disorder and/or the neuro-psychiatric disorder is selected from, ADHD, ADD, OCD, anxiety, depression, a learning deficiency, an attention related deficiency or dysfunction, amnesia, a memory dysfunction, traumatic brain injury, stroke, dementia, neurodegenerative disorder, and any combinations thereof.

24. The method according to claim 19, wherein the user is a normal user and wherein the step of stimulating augments the cognitive performance of the user as compared to the cognitive performance of the user when the step of stimulating is not performed.

25. The method according to claim 19, wherein the one or more cortical regions comprise one or more of prefrontal cortex (PFC), a part of the PFC, a dorsolateral prefrontal cortex (DLPFC), a part of the DLPFC, a temporoparietal cortex (TPC), a part of the TPC, an inferior frontal gyms (IFG), a part of the IFG, the temporal parietal junction (TPJ), a part of the TPJ, and any combinations thereof.

26. The method according to claim 19, wherein the step of sensing also includes sensing signals associated with neuronal activity in one or more additional cortical regions selected from, a visual cortical region, a region of the primary visual cortex (V1), the medial temporal lobe of the visual cortex, a region of a motor cortex, a region of a pre-motor cortex, a region of a somato-sensory cortex, a region of a auditory cortex, a mesial surface of a right cortical occipital lobe, the associative cortex, other areas of the visual cortex, an auditory cortex, a motor cortex, BA 17, BA 18, BA 19, BA 7, BA 6, BA 5, BA 4 and any combinations thereof, and wherein the step of processing also includes processing the signals sensed in the additional cortical regions to detect the indication associated with an intention to perform a cognitive task and/or the presentation of a cognitive task, and/or performing the cognitive task.

27. The method according to claim 20, wherein the one or more deep brain structures are selected from ventral tegmental area (VTA), striatum, caudate nucleus, putamen, nucleus accumbens (NA), locus ceruleus, hippocampus, amygdala, a deep brain structure of the meso-limbic system, a deep brain structure functionally participating in enhancing and/or facilitating learning, memory and attention focusing, a subcortical region of the brain, a substantia nigra, a dorsal striatum, a part of the limbic structures within a mesocortical system, a part of a nigrostriatal system, a part of teberoinfundibular system, fornix, nucleus basalis of Meynert (NBM), anterior caudate nucleus, dorsal striatum, anterior thalamic nucleus, central thalamus, lateral hypothalamus, subgenual cingulated region (BA 25), enthorinal cortex, perforant path, medial frontal lobe, subthalamic nucleus and any combinations thereof.

28. The method according to claim 19, wherein the step of stimulating is selected from,
stimulating one or more deep brain structures for enhancing cognitive performance of the user,
stimulating one or more deep brain structures and one or more cortical regions for enhancing cognitive performance of the user, and
stimulating one or more cortical regions for enhancing cognitive performance of the user.

29. The method according to claim 19, wherein the step of stimulating comprises stimulating one or more cortical regions selected from a prefrontal cortex (PFC), a part of the PFC, a dorsolateral prefrontal cortex (DLPFC), a part of the DLPFC, a temporoparietal cortex (TPC), a part of the TPC, an inferior frontal gyrus (IFG), a part of the IFG, the temporal parietal junction (TPJ), a part of the TPJ, and any combinations thereof for enhancing and/or augmenting and/or improving cognitive performance of the user.

30. The method according to claim 19, wherein the steps of sensing, processing and stimulating are performed automatically.

31. The method according to claim 19, wherein the performing of one or more steps selected from the steps of sensing, processing and stimulating is user controlled.

32. The method according to claim 19, wherein the method also includes the steps of,
stimulating the visual cortex of the user to cause the user to perceive a virtual image of a graphic user interface (GUI),
sensing in the motor cortex of the user signals associated with a voluntary intention to perform a movement or the imagining of performing a movement or the performing of a movement, and
processing the signals sensed in the motor cortex to perform an interaction with the virtual image of the GUI for controlling the performing of one or more steps selected from the steps of sensing, processing and stimulating.

33. The method according to claim 19, wherein the step of processing comprises processing the signals using a method selected from, kernel analysis, principal component analysis, spectral analysis methods, common spatial patterns method (CSP), Analytic CSP (ACSP), time domain analytic methods, Frequency Domain analytic methods, supervised pattern classification, cluster seeking methods, likelihood functions and statistical decision.

34. The method according to claim 19, wherein the steps of sensing and stimulating are performed in a dorsolateral prefrontal cortex (DLPFC).

35. The method according to claim 19, wherein the step of processing comprises computing Fourier Transform (FT) of the sensed signals to obtain power spectra data for multiple electrode pairs, performing phase coupling analysis on the data to compute a weighted phase lag index (wPLI), comparing the computed wPLI to a threshold value and initiating the step of stimulating the one or more target brain regions of the user upon detecting that the computed wPLI is smaller than a threshold value.

36. The method according to claim 35, wherein the step of initiating the step of stimulating comprises initiating the step of stimulating after a time delay period starting at the time of the detecting.

37. The method according to claim 35, wherein the step of stimulating comprises stopping the sensing for the duration of the step of stimulating.

38. The method according to claim 19, wherein the step of processing comprises, computing Fourier Transform (FT) of the sensed signals to obtain power spectra data, computing from the power spectra the spectral power in the gamma frequency band (Pγ) value of value of, comparing the computed Pγ to a threshold value and initiating the step of stimulating upon detecting that Pγ is smaller than or equal to a threshold value.

39. The method according to claim 38, wherein the step of initiating the step of stimulating comprises initiating the step of stimulating after a time delay period starting at the time of the detecting.

40. The method according to claim 38, wherein the step of stimulating comprises stopping the sensing for the duration of the step of stimulating.

41. A brain computer interface (BCI) system for augmenting and/or assisting and/or improving cognitive performance of a user, the system comprising:
one or more sensing devices for sensing signals associated with neuronal electrical activity in one or more cortical regions of the user;
one or more stimulating devices for providing stimulating signals to one or more target brain regions selected from the group consisting of one or more deep brain structures of the user, one or more cortical regions of the user and a combination of at least one cortical region and at least one deep brain structure of the user;
at least one processor/controller in communication with the one or more sensing devices and the one or more stimulating devices, the at least one processor/controller is programmed to process signals sensed in the one or more cortical regions for detecting an indication associated with an intention of the user to perform a cognitive task and/or a presentation of a cognitive task to the user and/or performing of the cognitive task by the user, and to control the stimulating of the one or more target brain regions responsive to detecting the indication for augmenting and/or assisting, and/or improving the cognitive performance of the user, and
at least one power source for energizing the BCI system.

42. The BCI system according to claim 41, wherein the one or more sensing devices comprise electrodes configured to sense electrical signals associated with electrical activity in the one or more cortical regions.

43. The BCI system according to claim 41, wherein the one or more stimulating devices comprise electrodes configured to apply electrical stimulating signals to the target brain regions.

44. The BCI system according to claim 41 wherein at least one sensing device of the one or more sensing devices comprises one or more electrode sets configured to sense electrical signals associated with electrical activity in the one or more cortical regions and wherein at least one stimulating device of the one or more stimulating devices comprises one or more electrode sets configured to apply electrical signals to the one or more target brain regions for electrically stimulating the one or more target brain regions.

45. The BCI system according to claim 41, wherein the indication is selected from,
a phase alteration of the sensed signals in one or more frequency bands,
an alteration in computed spectral power of the sensed signals in the one or more frequency bands, and
any combination thereof.

46. The BCI system according to claim 45, wherein the frequency band is selected from, delta band, theta, mu, alpha, beta, and gamma band, or any combinations thereof.

47. The method according to claim 19, wherein the indication is selected from,
a phase alteration of the sensed signals in one or more frequency bands,
an alteration in computed spectral power of the sensed signals in the one or more frequency bands, and
any combinations thereof.

48. The method according to claim 47, wherein the frequency band is selected from, delta band, theta, mu, alpha, beta, and gamma band, or any combinations thereof.

49. The BCI system according to claim 41, wherein the user is selected from a normal user and a user having a neurological disorder, a psychiatric disorder, or a neuro-psychiatric disorder.

50. The BCI system according to claim 41, wherein the user is a normal user and wherein the providing of stimulating signals augments the cognitive performance of the user as compared to the cognitive performance of the user when the providing of stimulating signals is not performed.

51. The BCI system according to claim 1, wherein the user is a normal user and wherein the stimulating of the one or more target brain regions augments the cognitive performance of the user as compared to the cognitive performance of the same user when the one or more target brain regions are not stimulated.
